SEQUENCE LISTING (163 sequences)

SEQ ID NO: 1 - DNA, 21 bp, Artificial Sequence
  synthetic construct
taggtaatctgaggtggcatc

SEQ ID NO: 2 - DNA, 28 bp, Artificial Sequence
  synthetic construct
tgtaatttcctacctaagtagttacagt

SEQ ID NO: 3 - DNA, 19 bp, Artificial Sequence
  synthetic construct
ggtgattatgagagaacaa

SEQ ID NO: 4 - DNA, 22 bp, Artificial Sequence
  synthetic construct
tggaggaaaagtggtaatgaga

SEQ ID NO: 5 - DNA, 17 bp, Artificial Sequence
  synthetic construct
cagcagaaggaacagga

SEQ ID NO: 6 - DNA, 33 bp, Artificial Sequence
  synthetic construct
actcagagagactacgagccagctttaagcaac

SEQ ID NO: 7 - DNA, 19 bp, Artificial Sequence
  synthetic construct
cagcctcacttttgaacac

SEQ ID NO: 8 - DNA, 18 bp, Artificial Sequence
  synthetic construct
gctgacagggaggaaaac

SEQ ID NO: 9 - DNA, 18 bp, Artificial Sequence
  synthetic construct
tctccaacaaaccagagt

SEQ ID NO: 10 - DNA, 20 bp, Artificial Sequence
  synthetic construct
tgtctgactgctgatttgat

SEQ ID NO: 11 - DNA, 17 bp, Artificial Sequence
  synthetic construct
gagtggccatgtaggat

SEQ ID NO: 12 - DNA, 27 bp, Artificial Sequence
  synthetic construct
agaagagacgacaacgctgtgaggctc

SEQ ID NO: 13 - DNA, 24 bp, Artificial Sequence
  synthetic construct
cagaaatgagattcatttgctgga

SEQ ID NO: 14 - DNA, 18 bp, Artificial Sequence
  synthetic construct
gtctaggccacttccctc

SEQ ID NO: 15 - DNA, 18 bp, Artificial Sequence
  synthetic construct
tctttgagtgtcaatttc

SEQ ID NO: 16 - DNA, 20 bp, Artificial Sequence
  synthetic construct
gccctgtttcaagttatctg

SEQ ID NO: 17 - DNA, 17 bp, Artificial Sequence
  synthetic construct
ttgggccagtctagcag

SEQ ID NO: 18 - DNA, 27 bp, Artificial Sequence
  synthetic construct
tccagtgactctgctattatggtaagt

SEQ ID NO: 19 - DNA, 24 bp, Artificial Sequence
  synthetic construct
gcgttagtcagtcttaccctaaac

SEQ ID NO: 20 - DNA, 21 bp, Artificial Sequence
  synthetic construct
tacacgagtttcgttctttgc

SEQ ID NO: 21 - DNA, 30 bp, Artificial Sequence
  synthetic construct
gcacattccgggagggcgttatgggcactg

SEQ ID NO: 22 - DNA, 19 bp, Artificial Sequence
  synthetic construct
tagaagaacacagtggggc

SEQ ID NO: 23 - DNA, 21 bp, Artificial Sequence
  synthetic construct
gctggattgaagtgcatttga

SEQ ID NO: 24 - DNA, 19 bp, Artificial Sequence
  synthetic construct
caaaacaaaacaggattca

SEQ ID NO: 25 - DNA, 22 bp, Artificial Sequence
  synthetic construct
gttaggaagccaaataggatgt

SEQ ID NO: 26 - DNA, 20 bp, Artificial Sequence
  synthetic construct
cacttacactagaatgagca

SEQ ID NO: 27 - DNA, 29 bp, Artificial Sequence
  synthetic construct
cacgaggaaggaagggaagacatgaccca

SEQ ID NO: 28 - DNA, 20 bp, Artificial Sequence
  synthetic construct
tacaagcacaaatgaacaag

SEQ ID NO: 29 - DNA, 22 bp, Artificial Sequence
  synthetic construct
tttactgtaattccactccact

SEQ ID NO: 30 - DNA, 38 bp, Artificial Sequence
  synthetic construct
gggcatttgactgacagagtaggggacagtcggagagc

SEQ ID NO: 31 - DNA, 22 bp, Artificial Sequence
  synthetic construct
cattagttatgtccactattca

SEQ ID NO: 32 - DNA, 20 bp, Artificial Sequence
  synthetic construct
tcttgcaataaccctcacag

SEQ ID NO: 33 - DNA, 22 bp, Artificial Sequence
  synthetic construct
agtaaggaaagtaattatttca

SEQ ID NO: 34 - DNA, 20 bp, Artificial Sequence
  synthetic construct
ggttaccaagaccagatgga

SEQ ID NO: 35 - DNA, 18 bp, Artificial Sequence
  synthetic construct
gcacacatgcacatgagt

SEQ ID NO: 36 - DNA, 21 bp, Artificial Sequence
  synthetic construct
gctctctctcagttgggactt

SEQ ID NO: 37 - DNA, 17 bp, Artificial Sequence
  synthetic construct
ccacatctcctccagca

SEQ ID NO: 38 - DNA, 18 bp, Artificial Sequence
  synthetic construct
caaagggatgggttcctc

SEQ ID NO: 39 - DNA, 23 bp, Artificial Sequence
  synthetic construct
cgcagctacaaatgtacactgcg

SEQ ID NO: 40 - DNA, 18 bp, Artificial Sequence
  synthetic construct
taggtgtgaacgagcctg

SEQ ID NO: 41 - DNA, 17 bp, Artificial Sequence
  synthetic construct
cctgttagagctcccac

SEQ ID NO: 42 - DNA, 27 bp, Artificial Sequence
  synthetic construct
cggtccccagccctgtagccacgaccg

SEQ ID NO: 43 - DNA, 20 bp, Artificial Sequence
  synthetic construct
tagtctcagtggactttggt

SEQ ID NO: 44 - DNA, 21 bp, Artificial Sequence
  synthetic construct
caaacatcaaacaattcagca

SEQ ID NO: 45 - DNA, 26 bp, Artificial Sequence
  synthetic construct
acctgagaatgtggttacttgcaggt

SEQ ID NO: 46 - DNA, 18 bp, Artificial Sequence
  synthetic construct
tccccacccagaagaaac

SEQ ID NO: 47 - DNA, 19 bp, Artificial Sequence
  synthetic construct
gggaggagaaggactgatg

SEQ ID NO: 48 - DNA, 33 bp, Artificial Sequence
  synthetic construct
cctcagctgtcctccccacttccgtcactgagg

SEQ ID NO: 49 - DNA, 19 bp, Artificial Sequence
  synthetic construct
ccccagtaatggcagatca

SEQ ID NO: 50 - DNA, 20 bp, Artificial Sequence
  synthetic construct
tgccttccagatatgcattc

SEQ ID NO: 51 - DNA, 20 bp, Artificial Sequence
  synthetic construct
acagcaagtcaattcactgt

SEQ ID NO: 52 - DNA, 19 bp, Artificial Sequence
  synthetic construct
ctccagaatcaagctgtgt

SEQ ID NO: 53 - DNA, 19 bp, Artificial Sequence
  synthetic construct
tcatgtaggagtgcattgt

SEQ ID NO: 54 - DNA, 24 bp, Artificial Sequence
  synthetic construct
ccagtaagacagctgtacactggt

SEQ ID NO: 55 - DNA, 20 bp, Artificial Sequence
  synthetic construct
cgtatcattcggttatcaag

SEQ ID NO: 56 - DNA, 19 bp, Artificial Sequence
  synthetic construct
cccatctgagcaaagaact

SEQ ID NO: 57 - DNA, 30 bp, Artificial Sequence
  synthetic construct
aatcggccggatttccctccaggtaccgat

SEQ ID NO: 58 - DNA, 25 bp, Artificial Sequence
  synthetic construct
taatttctctatgctcataggttct

SEQ ID NO: 59 - DNA, 21 bp, Artificial Sequence
  synthetic construct
ttcaaacctcctattccacag

SEQ ID NO: 60 - DNA, 26 bp, Artificial Sequence
  synthetic construct
acagcacatgtaacatatggagtgct

SEQ ID NO: 61 - DNA, 19 bp, Artificial Sequence
  synthetic construct
gcacaggcaattgagaaga

SEQ ID NO: 62 - DNA, 19 bp, Artificial Sequence
  synthetic construct
ctcctttaaaagggtcggt

SEQ ID NO: 63 - DNA, 32 bp, Artificial Sequence
  synthetic construct
acagcccatttgtttctcctgtcttgaggctg

SEQ ID NO: 64 - DNA, 23 bp, Artificial Sequence
  synthetic construct
ccaaactcctggatcataaaaca

SEQ ID NO: 65 - DNA, 23 bp, Artificial Sequence
  synthetic construct
ggaatcagggataatctctatca

SEQ ID NO: 66 - DNA, 18 bp, Artificial Sequence
  synthetic construct
tccagggtgcttacactg

SEQ ID NO: 67 - DNA, 20 bp, Artificial Sequence
  synthetic construct
tctaccgtctaacctgcaag

SEQ ID NO: 68 - DNA, 18 bp, Artificial Sequence
  synthetic construct
atctacgcctgagggaca

SEQ ID NO: 69 - DNA, 29 bp, Artificial Sequence
  synthetic construct
tgctgcctgagtgatgataagtgtcagca

SEQ ID NO: 70 - DNA, 22 bp, Artificial Sequence
  Tag2
gtcgcaagcactcacgtagaga

SEQ ID NO: 71 - DNA, 20 bp, Artificial Sequence
  Tag1
tcgcaagcactcacgtagag

SEQ ID NO: 72 - DNA, 20 bp, Artificial Sequence
  synthetic construct
aggtaatctgaggtggcatc

SEQ ID NO: 73 - DNA, 28 bp, Artificial Sequence
  synthetic construct
tgtaatttcctacctaagtagttacagt

SEQ ID NO: 74 - DNA, 24 bp, Artificial Sequence
  synthetic construct
ggtgattatgagagaacaaccttc

SEQ ID NO: 75 - DNA, 22 bp, Artificial Sequence
  synthetic construct
ggtgattatgagaacaaccttc

SEQ ID NO: 76 - DNA, 30 bp, Artificial Sequence
  synthetic construct
atggaaaatgtaatatttctgaatgaaaga

SEQ ID NO: 77 - DNA, 21 bp, Artificial Sequence
  synthetic construct
cctttcatctaaatgcgttgc

SEQ ID NO: 78 - DNA, 23 bp, Artificial Sequence
  synthetic construct
ttgaaactcagagagactacgag

SEQ ID NO: 79 - DNA, 20 bp, Artificial Sequence
  synthetic construct
tgaaactcagactacgagcc

SEQ ID NO: 80 - DNA, 21 bp, Artificial Sequence
  synthetic construct
cgctgggtcatctattaacac

SEQ ID NO: 81 - DNA, 22 bp, Artificial Sequence
  synthetic construct
gaatgccagtattcacaacagt

SEQ ID NO: 82 - DNA, 24 bp, Artificial Sequence
  synthetic construct
acaaaccagagtcttcttatgaag

SEQ ID NO: 83 - DNA, 20 bp, Artificial Sequence
  synthetic construct
tccaacaaaccagtcttctt

SEQ ID NO: 84 - DNA, 22 bp, Artificial Sequence
  synthetic construct
tccaatccagtgtttcttctga

SEQ ID NO: 85 - DNA, 17 bp, Artificial Sequence
  synthetic construct
cacccagacaagccacc

SEQ ID NO: 86 - DNA, 19 bp, Artificial Sequence
  synthetic construct
gacaacgctgtgaggctct

SEQ ID NO: 87 - DNA, 18 bp, Artificial Sequence
  synthetic construct
cgacaacgctgaggctct

SEQ ID NO: 88 - DNA, 23 bp, Artificial Sequence
  synthetic construct
agaaatgagattcatttgctgga

SEQ ID NO: 89 - DNA, 18 bp, Artificial Sequence
  synthetic construct
gtctaggccacttccctc

SEQ ID NO: 90 - DNA, 23 bp, Artificial Sequence
  synthetic construct
cacctctttgagtgtcaatttcc

SEQ ID NO: 91 - DNA, 22 bp, Artificial Sequence
  synthetic construct
cacctctgagtgtcaatttccc

SEQ ID NO: 92 - DNA, 25 bp, Artificial Sequence
  synthetic construct
aattacacatccctcatttatccag

SEQ ID NO: 93 - DNA, 26 bp, Artificial Sequence
  synthetic construct
tgcaattaaaatctattgagcaatgg

SEQ ID NO: 94 - DNA, 23 bp, Artificial Sequence
  synthetic construct
ctgctattatggtaagtgtcgga

SEQ ID NO: 95 - DNA, 21 bp, Artificial Sequence
  synthetic construct
gctattatggtgtcggattca

SEQ ID NO: 96 - DNA, 23 bp, Artificial Sequence
  synthetic construct
cgttagtcagtcttaccctaaac

SEQ ID NO: 97 - DNA, 20 bp, Artificial Sequence
  synthetic construct
acacgagtttcgttctttgc

SEQ ID NO: 98 - DNA, 17 bp, Artificial Sequence
  synthetic construct
cggcacattccgggagg

SEQ ID NO: 99 - DNA, 15 bp, Artificial Sequence
  synthetic construct
actgcccggctccgg

SEQ ID NO: 100 - DNA, 18 bp, Artificial Sequence
  synthetic construct
agaagaacacagtggggc

SEQ ID NO: 101 - DNA, 21 bp, Artificial Sequence
  synthetic construct
gctggattgaagtgcatttga

SEQ ID NO: 102 - DNA, 24 bp, Artificial Sequence
  synthetic construct
ggacaacaaaacaaaacaggattc

SEQ ID NO: 103 - DNA, 21 bp, Artificial Sequence
  synthetic construct
gggacaacaaaacaggattca

SEQ ID NO: 104 - DNA, 26 bp, Artificial Sequence
  synthetic construct
catttaggaagccaaataggatgtac

SEQ ID NO: 105 - DNA, 23 bp, Artificial Sequence
  synthetic construct
gtaaaagtctgcagaaaatgggt

SEQ ID NO: 106 - DNA, 19 bp, Artificial Sequence
  synthetic construct
acgaggaaggaagggaaga

SEQ ID NO: 107 - DNA, 19 bp, Artificial Sequence
  synthetic construct
cacgaggaagggaagacat

SEQ ID NO: 108 - DNA, 17 bp, Artificial Sequence
  synthetic construct
tgcatccttgctgacga

SEQ ID NO: 109 - DNA, 25 bp, Artificial Sequence
  synthetic construct
agcttttatttcagatacctgttga

SEQ ID NO: 110 - DNA, 26 bp, Artificial Sequence
  synthetic construct
tgcagtaactacaagtaaggaaagta

SEQ ID NO: 111 - DNA, 25 bp, Artificial Sequence
  synthetic construct
tgcagtaactacaaggaaagtaatt

SEQ ID NO: 112 - DNA, 20 bp, Artificial Sequence
  synthetic construct
cccactgatcatctcccaaa

SEQ ID NO: 113 - DNA, 24 bp, Artificial Sequence
  synthetic construct
cactatggtgattcctagtacctt

SEQ ID NO: 114 - DNA, 21 bp, Artificial Sequence
  synthetic construct
gtgttgctctctctcagttgg

SEQ ID NO: 115 - DNA, 18 bp, Artificial Sequence
  synthetic construct
gttgctctctcagttggg

SEQ ID NO: 116 - DNA, 25 bp, Artificial Sequence
  synthetic construct
gcatgcatttcaaagtttatacctg

SEQ ID NO: 117 - DNA, 25 bp, Artificial Sequence
  synthetic construct
caaggagagcaataagtatgtatcg

SEQ ID NO: 118 - DNA, 22 bp, Artificial Sequence
  synthetic construct
catttgactgacagagtagggg

SEQ ID NO: 119 - DNA, 20 bp, Artificial Sequence
  synthetic construct
agtgggcatttgacagagta

SEQ ID NO: 120 - DNA, 18 bp, Artificial Sequence
  synthetic construct
agcagatccttggtcagt

SEQ ID NO: 121 - DNA, 19 bp, Artificial Sequence
  synthetic construct
caaagggatgggttcctct

SEQ ID NO: 122 - DNA, 19 bp, Artificial Sequence
  synthetic construct
gcagctacaaatgtacact

SEQ ID NO: 123 - DNA, 19 bp, Artificial Sequence
  synthetic construct
gcagctacaaatatacact

SEQ ID NO: 124 - DNA, 24 bp, Artificial Sequence
  synthetic construct
tccaccataaatctcaactattcg

SEQ ID NO: 125 - DNA, 17 bp, Artificial Sequence
  synthetic construct
gctcccacaaccttcct

SEQ ID NO: 126 - DNA, 16 bp, Artificial Sequence
  synthetic construct
gttgccctggtcatgg

SEQ ID NO: 127 - DNA, 16 bp, Artificial Sequence
  synthetic construct
gttgccctggtcgtgg

SEQ ID NO: 128 - DNA, 24 bp, Artificial Sequence
  synthetic construct
gcattagctgaatcctttaagaga

SEQ ID NO: 129 - DNA, 23 bp, Artificial Sequence
  synthetic construct
aatccttaaaaacaatgcagcag

SEQ ID NO: 130 - DNA, 22 bp, Artificial Sequence
  synthetic construct
ctgagaatgttgttacttgcag

SEQ ID NO: 131 - DNA, 22 bp, Artificial Sequence
  synthetic construct
ctgagaatgtggttacttgcag

SEQ ID NO: 132 - DNA, 19 bp, Artificial Sequence
  synthetic construct
gaaaccttgccatctccag

SEQ ID NO: 133 - DNA, 18 bp, Artificial Sequence
  synthetic construct
ggcatcagtgacggaagt

SEQ ID NO: 134 - DNA, 18 bp, Artificial Sequence
  synthetic construct
gttcccagctctcctccc

SEQ ID NO: 135 - DNA, 17 bp, Artificial Sequence
  synthetic construct
gttcccagctgtcctcc

SEQ ID NO: 136 - DNA, 19 bp, Artificial Sequence
  synthetic construct
tctgctcagtgtgacaagt

SEQ ID NO: 137 - DNA, 27 bp, Artificial Sequence
  synthetic construct
gagtgtgatttgatttttatgcttttg

SEQ ID NO: 138 - DNA, 22 bp, Artificial Sequence
  synthetic construct
tgtgaattgacttgctgaggaa

SEQ ID NO: 139 - DNA, 19 bp, Artificial Sequence
  synthetic construct
tgaattgacttggtgagga

SEQ ID NO: 140 - DNA, 21 bp, Artificial Sequence
  synthetic construct
gttacagatattcccagagca

SEQ ID NO: 141 - DNA, 20 bp, Artificial Sequence
  synthetic construct
ttctcccaattctcaaagca

SEQ ID NO: 142 - DNA, 19 bp, Artificial Sequence
  synthetic construct
cagtaaggcagctgtacac

SEQ ID NO: 143 - DNA, 21 bp, Artificial Sequence
  synthetic construct
cagtaagacagctgtacactg

SEQ ID NO: 144 - DNA, 24 bp, Artificial Sequence
  synthetic construct
cattcggttatcaagtattaccca

SEQ ID NO: 145 - DNA, 20 bp, Artificial Sequence
  synthetic construct
ctttctggctcatgtctgac

SEQ ID NO: 146 - DNA, 19 bp, Artificial Sequence
  synthetic construct
gatttccctgcaggtacct

SEQ ID NO: 147 - DNA, 20 bp, Artificial Sequence
  synthetic construct
cggatttccctccaggtacc

SEQ ID NO: 148 - DNA, 29 bp, Artificial Sequence
  synthetic construct
gtctttaaggatgttctctaaatttttgt

SEQ ID NO: 149 - DNA, 24 bp, Artificial Sequence
  synthetic construct
acctcctattccacagaagattat

SEQ ID NO: 150 - DNA, 20 bp, Artificial Sequence
  synthetic construct
agcacatgtaacatatggag

SEQ ID NO: 151 - DNA, 20 bp, Artificial Sequence
  synthetic construct
agcacatgtaacataaggag

SEQ ID NO: 152 - DNA, 22 bp, Artificial Sequence
  synthetic construct
atgatctgaacagagcttctga

SEQ ID NO: 153 - DNA, 19 bp, Artificial Sequence
  synthetic construct
ggtctgagttcacctcctc

SEQ ID NO: 154 - DNA, 17 bp, Artificial Sequence
  synthetic construct
ctcaagacaggagaaac

SEQ ID NO: 155 - DNA, 19 bp, Artificial Sequence
  synthetic construct
cctcaagacaagagaaaca

SEQ ID NO: 156 - DNA, 23 bp, Artificial Sequence
  synthetic construct
ccaaactcctggatcataaaaca

SEQ ID NO: 157 - DNA, 19 bp, Artificial Sequence
  synthetic construct
caggaaaagagctgggtca

SEQ ID NO: 158 - DNA, 17 bp, Artificial Sequence
  synthetic construct
tccagggtgcttacact

SEQ ID NO: 159 - DNA, 18 bp, Artificial Sequence
  synthetic construct
atccagggtgctcacact

SEQ ID NO: 160 - DNA, 22 bp, Artificial Sequence
  synthetic construct
acaagctctctcatcctacatc

SEQ ID NO: 161 - DNA, 20 bp, Artificial Sequence
  synthetic construct
ccctgagtctgtctgatctg

SEQ ID NO: 162 - DNA, 20 bp, Artificial Sequence
  synthetic construct
tgcctgagtgatgataagtg

SEQ ID NO: 163 - DNA, 20 bp, Artificial Sequence
  synthetic construct
tgccttagtgatgataagtg

(19) United States
(12) Patent Application Publication
(10) Pub. No.: US 2024/0076739 A1
(43) Pub. Date: Mar. 7, 2024

(54) METHOD FOR DETECTING PRESENCE OR PROPORTION OF DONOR IN RECEPTOR SAMPLE, AND KIT

(71) Applicant: XIAMEN UNIVERSITY, Xiamen, Fujian (CN)

(72) Inventors: Qingge LI, Xiamen, Fujian (CN); Qiuying HUANG, Xiamen, Fujian (CN); Xinwen CHEN, Xiamen, Fujian (CN)

(21) Appl. No.: 18/267,840

(22) PCT Filed: Dec. 29, 2020
(86) PCT No.: PCT/CN2020/140651

§ 371 Date: Jun. 16, 2023

(30) Foreign Application Priority Data
Dec. 17, 2020 (CN) 202011494354.8

(51) Int. Cl. C12Q 1/6881; C12Q 1/6858

(52) U.S. Cl. CPC C12Q 1/6881; C12Q 1/6858; C12Q 2600/156; C12Q 2600/16

(57) ABSTRACT

A method for detecting SNP sites of a donor-derived sample and a recipient-derived sample, a method for detecting the presence or proportion of a donor in a receptor sample, and a kit for implementing the methods.

### TECHNICAL FIELD

The present application relates to the field of molecular diagnostics. Specifically, the present application relates to a method for detecting SNP sites in a donor-derived sample and a recipient-derived sample. Furthermore, the present application also relates to a method for detecting the presence or proportion of a donor in a recipient sample, and a kit for implementing the method.

### BACKGROUND ART

Heterologous DNA refers to a non-self DNA from one or more individuals that exist in the individual's own body. Compared with the individual's own DNA, the non-self DNA from one or more individuals can be defined as heterologous DNA. The most common example thereof is a donor-derived DNA that exists in the body of recipient during allogeneic transplantation. The current detection method for heterologous DNA can be applied to two major aspects, i.e., bone marrow transplantation and solid organ transplantation.

In bone marrow transplantation, allogeneic hematopoietic stem cell transplantation (Allo-HSCT) is the main treatment for many malignant hematological diseases and some non-malignant diseases. The detection method for the chimerism of hematopoietic stem cells after transplantation is mainly based on the polymorphic genetic markers in the population, such as red blood cell antigen, human leukocyte antigen typing, and short tandem repeat sequence analysis (STR-PCR). At present, the International Bone Marrow Transplant Registry Group has listed STR-PCR analysis technology as the gold standard for quantitative monitoring of donor cell chimerism after HSCT, but its defects include non-specific interference caused by competitive amplification, and stutter strips caused by polymerase slippage. Some studies have found that when the ratio of donor cells and recipient cells is below 5%-10%, the sensitivity will be significantly reduced (Bone Marrow Transplant, 2001,28(5):511-8). Chimera detection methods using other types of specific markers have been reported (J Mol Diagn, 2009,11(1):66-74), but there are still shortcomings such as low sample throughput, high cost of consumables, poor detection sensitivity, and complicated experimental operations.

At present, the monitoring of grafts after solid organ transplantation often uses blood for kidney and liver function tests, or uses puncture needles to collect tissues for pathological examination. As for the routine function tests by blood drawing, the sensitivity and specificity of various indicators such as creatinine, ALT, AST, bilirubin, etc. are not high, and cannot accurately reflect the condition of the graft. As for the tissue biopsy as the current gold standard, although it can directly reflect the condition of the graft, there are still problems such as infection or injury caused by invasive detection, injury lagging behind treatment when the abnormality is detected, and inaccurate sampling position of the punctured lesion, etc. The research of Stephen R. Quake et al. (Proc Natl Acad Sci USA. 2011; 108(15):6229-6234.) indicates that the proportion of heterologous donor-derived cell-free DNA (dd-cfDNA) can reflect the graft state to a certain extent.

At present, the detection of dd-cfDNA content is mostly based on human genetic polymorphism information (Sci Transl Med. 2014; 6(241):241ra77.), or based on changes in epigenetic modifications (Gut. 2018; 67(12):2204-2212.). Beck J et al. reported (Clin Chem, 2013,59(12):1732-41.), in an early postoperative study of liver, kidney and heart transplant patients, the donor-derived single nucleotide polymorphism information was analyzed by qPCR technology, and the dPCR technique was used to determine the proportion of dd-cfDNA in recipient plasma after transplantation. Grskovic et al. conducted the simultaneous detection of a large number of SNP sites by further development and improvement of the next-generation sequencing technology (NGS) platform, and obtained reliable verification results in the real-time monitoring of dd-cfDNA ratio in a large number of patients after heart transplantation (J Mol Diagn, 2016,18(6):890-902.). A Chinese invention patent application discloses a method for determining the donor-derived cfDNA ratio in a recipient cfDNA sample (CN106544407A), in which NGS is used to capture and sequence a target region to obtain a large number of SNP genotyping information of the recipient sample; and the recipient plasma cfDNA sample after transplantation is simultaneously subjected to target region capturing and sequencing to analyze the ratio of dd-cfDNA to total cfDNA. However, the above method still has the following problems when it is applied to the detection of heterologous genomic DNA or heterologous cell-free DNA: the NGS technical scheme is cumbersome in experimental operation, long in detection period (3-7 working days), high in detection cost, and thus is not suitable for regular monitoring after transplantation; while other conventional technologies have disadvantages such as low throughput, many operation steps, low detection sensitivity, and easy contamination when opening the cover.

### CONTENTS OF THE PRESENT INVENTION

In the present invention, unless otherwise specified, the scientific and technical terms used herein have the meanings commonly understood by those skilled in the art. Meanwhile, in order to better understand the present invention, definitions and explanations of relevant terms are provided below.

As used herein, the term “donor” refers to an individual who has provided or intends to provide an organ, tissue or cell for transplantation to another individual (recipient). In certain embodiments, the donor has provided or intends to provide an organ (e.g., kidney, heart, lung, liver, pancreas, or any combination thereof) for transplantation to another individual (recipient). In certain embodiments, the donor has provided or intends to provide another individual (recipient) with a hematopoietic stem cell (e.g., bone marrow hematopoietic stem cell, peripheral blood hematopoietic stem cell, cord blood hematopoietic stem cell) or a hematopoietic stem cell-containing tissue or organ (e.g., bone marrow) for transplantation.

As used herein, the term “recipient” refers to an individual who has received or intends to receive or be transplanted an organ, tissue or cell for transplantation provided by another individual (donor). In certain embodiments, the recipient has received or intends to receive or be transplanted an organ (e.g., kidney, heart, lung, liver, pancreas, or any combination thereof) provided by another individual (donor). In certain embodiments, the recipient has received or intends to receive or be transplanted a hematopoietic stem cell (e.g., bone marrow hematopoietic stem cell, peripheral blood hematopoietic stem cell, umbilical cord blood hematopoietic stem cell) or a hematopoietic stem cell-containing tissue or organ (e.g., bone marrow) from another individual (donor).

As used herein, the term “individual” refers to any biological entity. In certain embodiments, the individual is an animal individual, such as a mammalian (e.g., human, murine, rabbit, horse, sheep, etc.) individual.

As used herein, the term “donor chimerism rate” or “donor cell chimerism rate” refers to the phenomenon that donor and recipient cells migrate and coexist with each other after recipient receives an allograft or xenograft, which is a medical detection index that can be used to evaluate the curative effect of allogeneic hematopoietic stem cell transplantation, and its results have a warning effect on recurrence after transplantation and can prompt early clinical intervention.

As used herein, the term “donor cell-free DNA ratio” or “dd-cfDNA ratio” is a potential detection index that can be used to evaluate rejection reaction after organ transplantation, which is derived from the cell-free DNA released into the plasma during the cell necrosis and apoptosis of graft cells, and it results indicate the degree of graft damage and can guide early clinical intervention.

As used herein, the term “cluster analysis” refers to an analytical process of grouping a collection of physical or abstract objects into classes of similar objects. The goal of cluster analysis is to collect data for classification on the basis of similarity. Clustering has roots in many fields, including mathematics, computer science, statistics, biology, and economics. In different application areas, these techniques are used to describe data, measure the similarity between different data sources, and classify data sources into different clusters.

As used herein, the term “SNP (Single Nucleotide Polymorphism)” refers to a nucleic acid sequence polymorphism caused by variation of a single nucleotide at the genome level. The term “SNP site” refers to a site in the genome with a single nucleotide polymorphism. Herein, the SNP site includes a single site with single nucleotide polymorphism and a site with an insertion or deletion of one or more (e.g., 1, 2, 3, 4, 5, 6, or more) nucleotides. Herein, SNP site is named by its reference number (e.g., rs ID). The rs ID can be used to query the SNP site and its type in the public database, for example, through the dbSNP database of NCBI, ChinaMAP database, JSNP database, etc. In the present application, the selected or used SNP site is preferably a biallelic polymorphic SNP site.

As used herein, when referring to the “genotype” of an SNP site, it refers to a general term for the gene combination at the SNP site in all homologous chromosomes (usually two homologous chromosomes) of a certain organism. Herein, the “genotype” of SNP site refers to a gene combination at the SNP site in a pair of homologous chromosomes from the donor or the recipient. For example, “the genotype of the rs5858210 site of an individual is AG/-” means that a pair of homologous chromosomes of the individual have the nucleotide sequences “AG” and “-” (“−” means deletion) at the rs5858210 site, respectively. “The genotype of the rs5858210 site of an individual is AG/AG” means that a pair of homologous chromosomes of the individual both have the nucleotide sequence “AG” at the rs5858210 site. Correspondingly, a gene segment (i.e., nucleotide segment) containing the SNP site on a single chromosome is called an “allele” containing the SNP site. As used herein, for a certain SNP, different alleles usually have the completely identical nucleotide sequence except for the nucleotide difference at the SNP. When a pair of homologous chromosomes of an individual have the same nucleotide sequence (i.e., have the same allele) at a certain SNP site, the genotype of the individual at the SNP site is homozygous. When a pair of homologous chromosomes of an individual have different nucleotide sequences (i.e., have different alleles) at an SNP site, the genotype of the individual at the SNP site is heterozygous.

As used herein, the term “Fst” refers to a population fixation coefficient, which can reflect the level of heterozygosity of alleles in the population, and is used to measure the degree of population differentiation. The value of Fst is between 0 and 1. When Fst is 1, it indicates that the alleles are fixed and completely differentiated in each local population; when Fst is 0, it indicates that the genetic structure of different local populations is completely consistent, and there is no differentiation among populations. In the present application, the selected SNP sites preferably have Fst<0.01 among different races. These sites have little differentiation among different races, and the level of gene heterozygosity is close.

As used herein, the term “complementary” means that two nucleic acid sequences are capable of forming hydrogen bonds between each other according to the base pairing principle (Waston-Crick principle), thereby forming a duplex. In the present application, the term “complementary” includes “substantially complementary” and “completely complementary”. As used herein, the term “completely complementary” means that every base in one nucleic acid sequence is capable of pairing with a base in the other nucleic acid strand without a mismatch or gap. As used herein, the term “substantially complementary” means that most of the bases in one nucleic acid sequence are capable of pairing with bases in the other nucleic acid strand, and the existence of mismatches or gaps (e.g., mismatches or gaps of one or several nucleotides) is allowed. Generally, two nucleic acid sequences that are “complementary” (e.g., substantially complementary or completely complementary) will selectively/specifically hybridize or anneal and form a duplex under conditions that allow nucleic acid hybridization, annealing, or amplification. Accordingly, the term “non-complementary” means that two nucleic acid sequences cannot hybridize or anneal and form a duplex under conditions that allow nucleic acid hybridization, annealing or amplification. As used herein, the term “not completely complementary” means that the bases in one nucleic acid sequence cannot completely pair with the bases in the other nucleic acid strand, and there is at least one mismatch or gap.

As used herein, the terms “hybridization” and “annealing” refer to a process by which complementary single-stranded nucleic acid molecules form a double-stranded nucleic acid. In the present application, “hybridization” and “annealing” have the same meaning and are used interchangeably. Typically, two nucleic acid sequences that are completely or substantially complementary are capable of performing hybridization or annealing. The complementarity required for hybridization or annealing of two nucleic acid sequences depends on the hybridization conditions used, especially temperature.

As used herein, the term “PCR reaction” has a meaning commonly understood by those skilled in the art, which refers to a reaction (polymerase chain reaction) for amplifying a target nucleic acid using a nucleic acid polymerase and primers. As used herein, the term “multiplex amplification” refers to the amplification of multiple target nucleic acids in the same reaction system. As used herein, the term “asymmetric amplification” means that, in the amplified product obtained by amplifying target nucleic acids, the amounts of two complementary nucleic acid strands are not the same, and the amount of one nucleic acid strand is greater than that of the other nucleic acid strand.

As used herein, and as generally understood by those skilled in the art, the terms “forward” and “reverse” are merely used for convenience in describing and distinguishing two primers in a primer pair; they are relative terms, and has no special meaning.

As used herein, the term “melting curve analysis” has the meaning generally understood by those skilled in the art, which refers to a method for analyzing the presence or identity of a double-stranded nucleic acid molecule by measuring the melting curve of the double-stranded nucleic acid molecule, which is commonly used to assess the dissociation characteristics of a double-stranded nucleic acid molecules during heating. Methods for performing melting curve analysis are well known to those skilled in the art (see, for example, the Journal of Molecular Diagnostics 2009, 11(2): 93-101). In the present application, the terms “melting curve analysis” and “melting analysis” have the same meaning and are used interchangeably.

In certain preferred embodiments of the present application, melting curve analysis can be performed by using a detection probe labeled with a reporter group and a quencher group. Briefly, at ambient temperature, the detection probe is capable of forming a duplex with its complementary sequence through base pairing. In this case, the reporter group (e.g., a fluorophore) and the quencher group on the detection probe are separated from each other, and the quencher group cannot absorb the signal (e.g., a fluorescent signal) from the reporter group. At this time, it is possible to detect the strongest signal (e.g., fluorescent signal). As the temperature increases, the two strands of the duplex begin to dissociate (i.e., the detection probe gradually dissociates from its complementary sequence), and the dissociated detection probe assumes a single-stranded free-coil state. In this case, the reporter group (e.g., a fluorophore) and the quencher group on the dissociated detection probe are in close proximity to each other, and the signal (e.g., fluorescent signal) emitted by the reporter group (e.g., a fluorophore) is absorbed by the quencher group. Therefore, as the temperature increases, the detected signal (e.g., fluorescence signal) gradually becomes weaker. When the two strands of the duplex are completely dissociated, all the detection probes are in single-stranded free coiled state. In this case, all the signals (e.g., fluorescent signals) emitted by the reporter groups (e.g., fluorophores) on the detection probes are absorbed by the quencher groups. As a result, the signals (e.g., fluorescent signals) emitted by the reporter groups (e.g., fluorophores) are essentially undetectable. Therefore, by detecting the signal (e.g., fluorescent signal) emitted by the duplex containing the detection probe during the heating or cooling process, the hybridization and dissociation processes of the detection probe and its complementary sequence can be observed, and the curve of signal intensity as a function of temperature can be obtained. Further, by performing derivative analysis on the obtained curve, a curve with the rate of change of signal intensity as the ordinate and temperature as the abscissa can be obtained (i.e., a melting curve of duplex). The peak in the melting curve is the melting peak, and its corresponding temperature is the melting point (Tₘ) of the duplex. In general, the higher the match degree between the detection probe and the complementary sequence (e.g., fewer bases are mismatched and more bases are matched), the higher the Tm of the duplex. Thus, by detecting the Tₘ of the duplex, the presence and identity of the sequence in the duplex that is complementary to the detection probe can be determined. Herein, the terms “melting peak”, “melting point” and “Tₘ” have the same meaning and are used interchangeably.

Through in-depth research, the inventors of the present application have established a method for detecting SNP sites of a donor-derived sample and a recipient-derived sample by using multiple asymmetric PCR amplification and multicolor probe melting curve analysis. On this basis, combined with the digital PCR system, the present application has developed a method for detecting the presence and proportion of a donor in a recipient sample, as well as a kit for implementing the method.

Therefore, in one aspect, the present application provides a method for detecting a SNP site with different genotypes between a donor and a recipient, comprising the following steps:
(a) providing a first sample containing one or more target nucleic acids derived from the donor, and a second sample containing one or more target nucleic acids derived from the recipient, in which the target nucleic acid contains one or more candidate SNP sites, and,
providing a first universal primer and a second universal primer, and, for each candidate SNP site, providing at least one target-specific primer pair; wherein,
the first universal primer comprises a first universal sequence;
the second universal primer comprises a second universal sequence, the second universal sequence comprises the first universal sequence and additionally comprises at least one nucleotide at the 3′ end of the first universal sequence;
the target-specific primer pair is capable of performing amplification using the target nucleic acid as a template to generate a nucleic acid product containing the candidate SNP site, and the target-specific primer pair contains a forward primer and a reverse primer, wherein, the forward primer comprises the first universal sequence and a forward nucleotide sequence specific to the target nucleic acid, and the forward nucleotide sequence is located at the 3′ end of the first universal sequence; the reverse primer comprises the second universal sequence and a reverse nucleotide sequence specific to the target nucleic acid, and the reverse nucleotide sequence is located at the 3′ end of the second universal sequence; and, the second universal sequence is not completely complementary to a complementary sequence of the forward primer; and
(b) under a condition that allows nucleic acid amplification, amplifying the target nucleic acids in the first sample and the second sample, respectively, by using the first universal primer and the second universal primer and the target-specific primer pair, thereby obtaining amplification products respectively corresponding to the first sample and the second sample;
(c) performing melting curve analysis on the amplification products corresponding to the first sample and the second sample obtained in step (b);
(d) according to the result of the melting curve analysis of step (c), determining such an SNP site at which the first sample and the second sample have different genotypes.

In the method of the present application, the forward primer and the reverse primer respectively comprise a forward nucleotide sequence and a reverse nucleotide sequence specific to the target nucleic acid, thus, during the PCR reaction, the target-specific primer pair (forward primer and reverse primer) will anneal to the target nucleic acid and initiate PCR amplification, thereby producing an initial amplification product comprising two nucleic acid strands (nucleic acid strand A and nucleic acid strand B) that are complementary to the forward primer and the reverse primer, respectively. Further, since both the forward primer and the first universal primer contain the first universal sequence, the nucleic acid strand A complementary to the forward primer can also be complementary to the first universal primer. Similarly, the nucleic acid strand B complementary to the reverse primer can also be complementary to the second universal primer.

Therefore, as the PCR reaction proceeds, the first universal primer and the second universal primer will respectively anneal to the nucleic acid strand A and the nucleic acid strand B of the initial amplification product, and further initiate PCR amplification. In this process, since the reverse primer/second universal primer contains the first universal sequence, the first universal primer not only can anneal to the nucleic acid strand A (the nucleic acid strand complementary to the forward primer/first universal primer) and synthesize its complementary strand, but also can anneal to the nucleic acid strand B (the nucleic acid strand complementary to the reverse primer/second universal primer) and synthesize its complementary strand. That is, the first universal primer can simultaneously amplify the nucleic acid strand A and the nucleic acid strand B of the initial amplification product. At the same time, the second universal primer contains an additional nucleotide at the 3′ end of the first universal primer, although the second universal primer may also anneal to the nucleic acid strand A (the nucleic acid strand complementary to the forward primer/first universal primer, which has a sequence complementary to the forward primer), it does not match the nucleic acid strand A at the 3′ end (i.e., not completely complementary at the 3′ end). Thus, during the amplification process, the second universal primer will preferentially anneal to the nucleic acid strand B (the nucleic acid strand complementary to the reverse primer/second universal primer) and synthesize its complementary strand, while will substantially be unable to extend and synthesize the complementary strand of the nucleic acid strand A (the nucleic acid strand complementary to first forward primer/first universal primer).

Therefore, as the PCR amplification proceeds, the synthesis efficiency of the complementary strand (nucleic acid strand B) of the nucleic acid strand A will be significantly lower than that of the complementary strand (nucleic acid strand A) of the nucleic acid strand B, resulting in the complementary strand (nucleic acid strand A) of the nucleic acid strand B is synthesized and amplified in large quantities, while the synthesis and amplification of the complementary strand (nucleic acid strand B) of the nucleic acid strand A is inhibited, thereby resulting in a large amount of single-stranded product (nucleic acid strand A, which contains the sequence complementary to the forward primer/first universal primer and the sequence of the reverse primer/second universal primer) and realizing asymmetric amplification of the target nucleic acid containing one or more SNP sites. Thus, in steps (a) and (b) of the method of the present application, asymmetric amplification of one or more target nucleic acids in a sample is achieved.

In addition, since both the forward primer and the reverse primer contain the first universal sequence, during the PCR reaction, the primer dimers formed due to the non-specific amplification of the forward primer and the reverse primer will produce a single-stranded nucleic acid with reverse sequences complementary to each other contained at its 5′ end and 3′ end after denaturation, and the single-stranded nucleic acid is easy to self-anneal during the annealing stage to form a stable panhandle structure, which prevents the first universal primer and the second universal primer from performing annealing and extension of the single-stranded nucleic acid, thereby inhibiting further amplification of the primer dimers. Therefore, in the method of the present invention, non-specific amplification of primer dimers can be effectively suppressed.

In some embodiments, in step (d) of the method, the genotype of each candidate SNP site in the first sample and the second sample is determined according to the results of the melting curve analysis, thereby detecting an SNP site with different genotypes in the donor and the recipient.

In certain embodiments, the recipient has received or intends to receive or be transplanted an organ, tissue or cell from the donor.

In certain embodiments, the recipient has received or intends to receive or be transplanted an organ (e.g., kidney, heart, lung, liver, pancreas, or any combination thereof) from the donor.

In certain embodiments, the recipient has received or intends to receive or be transplanted with a hematopoietic stem cell (e.g., bone marrow hematopoietic stem cell, peripheral blood hematopoietic stem cell, umbilical cord blood hematopoietic stem cell, or any combination thereof) or a hematopoietic stem cell-containing tissue or organ (e.g., bone marrow) from the donor.

In certain embodiments, the second sample is substantially free of nucleic acids from the donor. In such embodiments, “substantially free of nucleic acids from the donor” means that there are no nucleic acids from the donor, or, the nucleic acid from the donor accounts for no more than 10% (e.g., no more than 5%, not more than 3%, not more than 1%, or lower) of the total nucleic acids in the second sample.

In certain embodiments, the first sample is from the donor; for example, the first sample comprises a cell or tissue from the donor; for example, the first sample is selected from skin, saliva, urine, blood, hair, nail, or any combination thereof from the donor.

In certain embodiments, the second sample is from the recipient (e.g., a recipient who has or has not undergone transplantation); for example, the second sample comprises a cell or tissue from the recipient; for example, the second sample is selected from skin, saliva, urine, blood, hair, nail, or any combination thereof from the recipient.

In certain embodiments, for the recipient who has not undergone transplantation, the second sample can be any cell or tissue (e.g., skin, saliva, urine, blood, etc.). For recipient who has undergone transplantation, the second sample contains substantially no nucleic acids from the donor.

In some preferred embodiments, for the recipient who has undergone hematopoietic stem cell transplantation, the second sample can be selected from skin, saliva, urine, hair, nail, or tissue, etc., but cannot be selected from blood, because the blood sample from the recipient who has undergone hematopoietic stem cell transplantation may contain a large amount of the nucleic acids from the donor. In some preferred embodiments, for the recipient who has undergone kidney transplantation, the second sample can be selected from skin, saliva, hair, nail, or tissue, etc., but cannot be selected from blood and urine, because the blood and urine samples from the recipient who has undergone kidney transplantation may contain a large amount of the nucleic acids form the donor. In some preferred embodiments, for the recipient who has undergone liver transplantation, the second sample can be selected from skin, saliva, hair, nail, urine, or tissue, etc., but cannot be selected from blood, because the blood sample from the recipient who has undergone kidney transplantation may contain a large amount of the nucleic acids from the donor.

In some embodiments, in step (a), for each candidate SNP site, a detection probe is also provided, the detection probe comprises a nucleotide sequence specific to the target nucleic acid and is capable of annealing or hybridizing to a region containing the candidate SNP site in the target nucleic acid, and the detection probe is labeled with a reporter group and a quencher group, wherein the reporter group is capable of sending a signal, and the quencher group is capable of absorbing or quenching the signal sent by the reporter group; and, the signal sent by the detection probe when it is hybridized to its complementary sequence is different from the signal sent when it is not hybridized to its complementary sequence;

Furthermore, in step (c), the detection probes are used to perform melting curve analysis on the amplification products corresponding to the first sample and the second sample obtained in step (b).

In certain embodiments, the first sample comprises DNA (e.g., genomic DNA).

In certain embodiments, the second sample comprises DNA (e.g., genomic DNA).

In a second aspect, the present application provides a method for detecting the presence or proportion of nucleic acids of a donor in a sample from a recipient who has undergone transplantation, wherein the method comprises the following steps:
(1) providing a nucleic acid-containing sample to be tested from the recipient who has been transplanted with a cell, tissue or organ from the donor;
(2) identifying one or more target SNP sites, wherein, at the target SNP site, the recipient has a first genotype comprising a first allele, and the donor has a second genotype comprising a second allele, wherein the first genotype is different from the second genotype and the first allele is different from the second allele;
(3) performing quantitative detection on the first allele and the second allele of each target SNP site in the sample to be tested, respectively; then, according to the results of the quantitative detection of the first allele and the second allele, determining the presence or proportion of the nucleic acids from the donor in the sample to be tested.

In some embodiments, in step (2), different alleles at the SNP site can be discriminated by a mechanism selected from: probe hybridization, primer extension, hybridization ligation and specific digestion. In some embodiments, in step (2), the target SNP site can be identified by a method selected from the following: sequencing method (e.g., first generation sequencing method, pyrosequencing method, second generation sequencing method), chip method (e.g., using a solid-phase chip or liquid-phase chip capable of detecting SNP), qPCR-based detection method (e.g., Tagman probe method), mass spectrometry (e.g., iPLEX™ Gold based on MassARRAY), chromatography (e.g., denaturing high performance liquid chromatography (dHPLC), electrophoresis (e.g., SNPshot method), detection method based on melting curve analysis. In certain embodiments, in step (2), the target SNP site is identified by a detection method based on multiplex PCR combined with melting curve analysis.

In certain embodiments, the target SNP site is identified by the method as previously described.

In some embodiments, in step (3), the first allele and the second allele of each target SNP site in the sample are quantitatively detected respectively by digital PCR.

In certain embodiments, step (3) is performed by the following scheme:
(I) selecting at least one (e.g., 1, 2, 3, or more) target SNP sites from step (2), and, for each selected target SNP site, providing an amplification primer set and a probe set, wherein,
(I-1) the amplification primer set comprises at least one amplification primer (e.g., a pair of amplification primers or more amplification primers), which can specifically amplify a nucleic acid molecule containing the target SNP site under a condition that allows acid hybridization or annealing;
(I-2) the probe set comprises a first probe and a second probe; wherein,
(i) the first probe and the second probe are each independently labeled with a reporter group and a quencher group, wherein the reporter group can emit a signal, and the quencher group is capable of absorbing or quenching the signal emitted by the reporter group; and, the first probe and the second probe are respectively labeled with different reporter groups (e.g., fluorophores); and
(ii) the first probe is capable of hybridizing or annealing (preferably completely complementary) to a nucleic acid molecule containing the first allele of the target SNP site, and the second probe is capable of hybridizing or annealing (preferably completely complementary) to a nucleic acid molecule containing the second allele of the target SNP site; and, the first probe and the second probe are specific for different alleles;
(II) performing digital PCR on the recipient sample using the amplification primer set and the probe set to quantitatively detect the nucleic acid molecule with the first allele and the nucleic acid molecule with the second allele;
(III) according to the quantitative detection results of step (II), determining the presence or proportion of the nucleic acids from the donor in the sample to be tested.

In certain embodiments, the first probe specifically anneals or hybridizes to a nucleic acid molecule having the first allele during the digital PCR reaction; and the second probe specifically anneals or hybridizes to a nucleic acid molecule having the second allele during the digital PCR reaction.

In certain embodiments, the first probe does not anneal or hybridize to a nucleic acid molecule having the second allele during the digital PCR reaction; and/or, the second probe does not anneal or hybridize to a nucleic acid molecule having the first allele during the digital PCR.

In some embodiments, before step (3), the sample to be tested from the recipient undergoes a pretreatment.

In certain embodiments, the pretreatment comprises extracting a nucleic acid from the sample and/or enriching nucleic acids in the sample (e.g., by concentration and/or amplification).

In certain embodiments, the recipient has received or transplanted with a hematopoietic stem cell (e.g., bone marrow hematopoietic stem cell, peripheral blood hematopoietic stem cell, umbilical cord blood hematopoietic stem cell, or any combination thereof) or a hematopoietic stem cell-containing tissue or organ (e.g., bone marrow) from the donor.

In certain embodiments, the sample to be tested comprises blood (e.g., peripheral blood) or component thereof (e.g., blood cell, plasma, monocyte, granulocyte, T cell, or any combination thereof) from the recipient after transplantation.

In certain embodiments, the target SNP site is an SNP site at which the recipient has a first genotype comprising a homozygous first allele, and, the donor has a second genotype comprising a homozygous second allele; or, the recipient has a first genotype comprising heterozygous first and second alleles, and the donor has a second genotype comprising a homozygous second allele.

In certain embodiments, the target SNP site is an SNP site at which the recipient has a first genotype comprising a homozygous first allele, and the donor has a second genotype comprising a homozygous second allele.

In certain embodiments, the ratio of donor in the recipient sample is calculated by one or more of the following methods:
(1) when the target SNP site is an SNP site at which the recipient has a first genotype (e.g., BB) containing a homozygous first allele, and the donor has a second genotype (e.g., AA) containing a homozygous second allele, the ratio of donor in the recipient sample is:

$$donor chimerism rate = 1 - \frac{{N}_{B}}{{N}_{A} + {N}_{B}}$$

wherein, N_{B} is the copy number of allele B (which can be determined by digital PCR), N_{A} is the copy number of allele A (which can be determined by digital PCR);
(2) when the target SNP site is an SNP site at which the recipient has a first genotype (e.g., AB) containing heterozygous first and second alleles, and the donor has a second genotype (e.g., AA) containing a homozygous second allele, the ratio of donor in the recipient sample is:

$$donor chimerism rate = 1 - \frac{2 {N}_{B}}{{N}_{A} + {N}_{B}}$$

wherein, N_{B} is the copy number of allele B (which can be determined by digital PCR), and N_{A} is the copy number of allele A (which can be determined by digital PCR).

In certain embodiments, wherein the recipient has received or transplanted with an organ (e.g., kidney, heart, lung, liver, pancreas, or any combination thereof) from the donor.

In certain embodiments, the recipient has received or transplanted with kidney from the donor.

In certain embodiments, the sample to be tested comprises blood (e.g., peripheral blood) or urine (especially in the case of kidney transplantation) from the recipient after transplantation.

In certain embodiments, the target SNP site is an SNP site at which the donor has a first genotype comprising a homozygous first allele, and the recipient has a second genotype comprising a homozygous second allele; or, the donor has a first genotype comprising heterozygous first and second alleles, and the recipient has a second genotype comprising a homozygous second allele.

In certain embodiments, the target SNP site is an SNP site at which the donor has a first genotype comprising a homozygous first allele, and the recipient has a second genotype comprising a homozygous second allele.

In certain embodiments, the ratio of donor in the recipient sample is calculated by one or more of the following methods:
(1) when the target SNP site is an SNP site at which the donor has a first genotype (e.g., BB) containing a homozygous first allele, and the recipient has a second genotype (e.g., AA) containing a homozygous second allele, the ratio of donor in the recipient sample is:

$$dd - cf DNA \% = \frac{{N}_{B}}{{N}_{A} + {N}_{B}}$$

wherein, N_{B} is the copy number of allele B (which can be determined by digital PCR), and N_{A} is the copy number of allele A (which can be determined by digital PCR);
(2) when the target SNP site is an SNP site at which the donor has a first genotype (e.g., AB) containing heterozygous first and second alleles, and the recipient has a second genotype (e.g., AA) containing a homozygous second allele, the ratio of donor in the recipient sample is:

$$dd - cf DNA \% = \frac{2 {N}_{B}}{{N}_{A} + {N}_{B}}$$

wherein, N_{B} is the copy number of allele B (which can be determined by digital PCR), N_{A} is the copy number of allele A (which can be determined by digital PCR).

In certain embodiments, wherein steps (a) to (b) of the method are performed by a scheme comprising steps (I) to (VI):
(I) providing the first sample, the second sample, the first universal primer and the second universal primer, as well as the target-specific primer pair; and optionally, the detection probe;
(II) mixing the sample with the first universal primer, the second universal primer, the target-specific primer pair, a nucleic acid polymerase, and optionally, the detection probe;
(III) incubating the product of the previous step under a condition that allows nucleic acid denaturation;
(IV) incubating the product of the previous step under a condition that allows nucleic acid annealing or hybridization;
(V) incubating the product of the previous step under a condition that allows nucleic acid extension; and
(VI) optionally, repeating steps (III) to (V) once or more times.

In certain embodiments, in step (III), the product of step (II) is incubated at a temperature of 80 to 105° C., thereby allowing the nucleic acid denaturation.

In certain embodiments, in step (III), the product of step (II) is incubated for 10 to 20 s, 20 to 40 s, 40 to 60 s, 1 to 2 min, or 2 to 5 min.

In certain embodiments, in step (IV), the product of step (III) is incubated at a temperature of 35 to 40° C., 40 to 45° C., 45 to 50° C., 50 to 55° C., 55 to 60° C., 60 to 65° C., or 65 to 70° C., thereby allowing the nucleic acid annealing or hybridization.

In certain embodiments, in step (IV), the product of step (III) is incubated for 10 to 20 s, 20 to 40 s, 40 to 60 s, 1 to 2 min, or 2 to 5 min.

In certain embodiments, in step (V), the product of step (IV) is incubated at a temperature of 35 to 40° C., 40 to 45° C., 45 to 50° C., 50 to 55° C., 55 to 60° C., 60 to 65° C., 65 to 70° C., 70 to 75° C., 75 to 80° C., 80 to 85° C., thereby allowing the nucleic acid extension.

In certain embodiments, in step (V), the product of step (IV) is incubated for 10 to 20 s, 20 to 40 s, 40 to 60 s, 1 to 2 min, 2 to 5 min, 5 to 10 min, 10 to 20 min or 20 to 30 min.

In certain embodiments, steps (IV) and (V) are performed at the same or different temperatures.

In certain embodiments, steps (III) to (V) are repeated at least once, for example, at least 2 times, at least 5 times, at least 10 times, at least 20 times, at least 30 times, at least 40 times, or at least 50 times. In certain embodiments, when steps (III) to (V) are repeated once or more times, the conditions used for each cycle of steps (III) to (V) are each independently the same or different.

In certain embodiments, the primers of the amplification primer set each independently have a length of 15 to 20 nt, 20 to 30 nt, 30 to 40 nt, 40 to 50 nt, 50 to 60 nt, 60 to 70 nt, 70 nt to 80 nt, 80 to 90 nt, 90 to 100 nt, 100 to 110 nt, 110 to 120 nt, 120 to 130 nt, 130 to 140 nt, 140 to 150 nt.

In certain embodiments, the primers of the amplification primer set, or any components thereof, each independently comprise or consist of naturally occurring nucleotides (e.g., deoxyribonucleotides or ribonucleotides), modified nucleotides, non-natural nucleotides, or any combination thereof.

In certain embodiments, the amplification primer set each independently comprises a primer pair having nucleotide sequences selected from the following or any combination thereof (e.g., combination of any 5 pairs, 10 pairs, 15 pairs, 20 pairs, 23 pairs): SEQ ID NOs: 72 and 73; 77 and 76; 80 and 81; 84 and 85; 88 and 89; 92 and 93; 96 and 97; 100 and 101; 104 and 105; 108 and 109; and 113; 116 and 117; 120 and 121; 124 and 125; 128 and 129; 132 and 133; 136 and 137; 140 and 141; 144 and 145; 148 and 149; 152 and 153; 156 and 157; 160 and 161.

In some embodiments, the first probe and the second probe each independently comprise or consist of naturally occurring nucleotides (e.g., deoxyribonucleotides or ribonucleotides), modified nucleotides, non-natural nucleotides (e.g., peptide nucleic acid (PNA) or locked nucleic acid), or any combination thereof.

In certain embodiments, the first probe and the second probe each independently have a length of 15 to 20 nt, 20 to 30 nt, 30 to 40 nt, 40 to 50 nt, 50 to 60 nt, 60 nt to 70 nt, 70 to 80 nt, 80 to 90 nt, 90 to 100 nt, 100 to 200 nt, 200 to 300 nt, 300 to 400 nt, 400 to 500 nt, 500 to 600 nt, 600 to 700 nt, 700 nt to 800 nt, 800 to 900 nt, 900 to 1000 nt.

In certain embodiments, the first probe and the second probe each independently have 3′-OH end; alternatively, the 3′-end of the probe is blocked; for example, the 3′-end of the probe is blocked by adding a chemical moiety (e.g., biotin or alkyl) to the 3′-OH of the last nucleotide of the probe, by removing the 3′-OH of the last nucleotide of the probe, or by replacing the last nucleotide with a dideoxynucleotide.

In certain embodiments, the first probe and the second probe each is independently a self-quenching probe; for example, the probe is labeled with a reporter group at or upstream of its 5′ end and labeled with a quencher group at or downstream of its 3′ end, or labeled with a reporter group at or downstream of its 3′ end and labeled with a quencher group at or upstream of its 5′ end. In certain embodiments, the reporter group and quencher group are separated by a distance of 10 to 80 nt or longer.

In certain embodiments, the reporter groups in the probes are each independently a fluorophore (e.g., ALEX-350, FAM, VIC, TET, CAL Fluor Gold 540, JOE, HEX, CAL Fluor Orange 560, TAMRA, CAL Fluor Red 590, ROX, CAL Fluor Red 610, TEXAS RED, CAL Fluor Red 635, Quasar 670, CY3, CY5, CY5.5, Quasar 705); and, the quencher group is a molecule or group (e.g., DABCYL, BHQ (e.g., BHQ-1 or BHQ-2), ECLIPSE, and/or TAMRA) capable of absorbing/quenching the fluorescence.

In certain embodiments, the first probe and the second probe are each independently linear, or have a hairpin structure.

In certain embodiments, the first probe and the second probe have different reporter groups. In certain embodiments, the first probe and the second probe are degradable by a nucleic acid polymerase (e.g., a DNA polymerase).

In certain embodiments, the probe set comprises probes having nucleotide sequences selected from the following or any combination thereof (e.g., any combination of 5, 10, 20, 40, 60 nucleotide sequences): SEQ ID NOs: 73, 74, 78, 79, 82, 83, 86, 87, 90, 91, 94, 95, 98, 99, 102, 103, 106, 107, 110, 111, 114, 115, 118, 119, 122, 123, 126, 127, 130, 131, 134, 135, 138, 139, 142, 143, 146, 147, 150, 151, 154, 155, 158, 159, 162, 163.

In a third aspect, the present application provides a method for identifying an SNP site having a first genotype comprising a homozygous first allele in a recipient, which comprises the following steps:
(a) providing a fifth sample from the recipient, in which the fifth sample contains one or more target nucleic acids derived from the recipient and is substantially free of nucleic acids derived from a donor; the target nucleic acid comprises one or more candidate SNP sites, and,
providing a first universal primer and a second universal primer, and, for each candidate SNP site, providing at least one target-specific primer pair; wherein,
the first universal primer comprises a first universal sequence;
the second universal primer comprises a second universal sequence, the second universal sequence comprises the first universal sequence and additionally comprises at least one nucleotide at the 3′ end of the first universal sequence;
the target-specific primer pair is capable of performing amplification using the target nucleic acid as a template to generate a nucleic acid product containing the candidate SNP site, and the target-specific primer pair comprises a forward primer and a reverse primer, wherein, the forward primer comprises the first universal sequence and a forward nucleotide sequence specific to the target nucleic acid, and the forward nucleotide sequence is located at the 3′ end of the first universal sequence; the reverse primer comprises the second universal sequence and a reverse nucleotide sequence specific to the target nucleic acid, and the reverse nucleotide sequence is located at the 3′ end of the second universal sequence; and, the second universal sequence is not completely complementary to the complementary sequence of the forward primer; and
(b) under a condition that allows nucleic acid amplification, amplifying the target nucleic acid in the fifth sample by using the first universal primer and the second universal primer and the target-specific primer pair, respectively, thereby obtaining amplification products corresponding to the fifth sample;
(c) performing melting curve analysis on the amplification products corresponding to the fifth sample obtained in step (b);
(d) according to the results of the melting curve analysis of step (c), identifying such an SNP site at which the recipient has a first genotype comprising a homozygous first allele.

In the method of the present application, the forward primer and the reverse primer are respectively comprises a forward nucleotide sequence and a reverse nucleotide sequence specific to the target nucleic acid, whereby, during the PCR reaction, the target-specific primer pair (forward primer and reverse primer) will anneal to the target nucleic acid, and initiates PCR amplification to produce an initial amplification product comprising two nucleic acid strands (nucleic acid strand A and nucleic acid strand B) complementary to the forward primer and the reverse primer, respectively. Further, since both the forward primer and the first universal primer contain the first universal sequence, the nucleic acid strand A complementary to the forward primer can also be complementary to the first universal primer. Similarly, the nucleic acid strand B complementary to the reverse primer can also be complementary to the second universal primer.

Therefore, as the PCR reaction proceeds, the first universal primer and the second universal primer will respectively anneal to the nucleic acid strand A and the nucleic acid strand B of the initial amplification product, and further initiate PCR amplification. In this process, since the reverse primer/second universal primer contains the first universal sequence, the first universal primer not only can anneal to the nucleic acid strand A (the nucleic acid strand complementary to the forward primer/first universal primer) and synthesize its complementary strand, but also can anneal to the nucleic acid strand B (the nucleic acid strand complementary to the reverse primer/second universal primer) and synthesize its complementary strand. That is, the first universal primer can simultaneously amplify the nucleic acid strand A and the nucleic acid strand B of the initial amplification product. At the same time, the second universal primer contains an additional nucleotide at the 3′ end of the first universal primer, although the second universal primer may also anneal to the nucleic acid strand A (the nucleic acid strand complementary to the forward primer/first universal primer, which has a sequence complementary to the forward primer), it does not match with the nucleic acid strand A at the 3′ end (i.e., not completely complementary at the 3′ end). Thus, during the amplification process, the second universal primer will preferentially anneal to the nucleic acid strand B (the nucleic acid strand complementary to the reverse primer/second universal primer) and synthesize its complementary strand, while substantially unable to perform extension to synthesize the complementary strand of the nucleic acid strand A (the nucleic acid strand complementary to first forward primer/first universal primer).

Therefore, as the PCR amplification proceeds, the synthesis efficiency of the complementary strand (nucleic acid strand B) of the nucleic acid strand A will be significantly lower than that of the complementary strand (nucleic acid strand A) of the nucleic acid strand B, so that the complementary strand (nucleic acid strand A) of the nucleic acid strand B is synthesized and amplified in large quantities, while the synthesis and amplification of the complementary strand (nucleic acid strand B) of the nucleic acid strand A is inhibited, thereby resulting in a large amount of single-stranded product (nucleic acid strand A, which contains the sequence complementary to the forward primer/first universal primer and the sequence of the reverse primer/second universal primer), and realizing the asymmetric amplification of the target nucleic acid containing one or more SNP sites. Thus, in steps (a) and (b) of the method of the present application, the asymmetric amplification of one or more target nucleic acids in the sample is achieved.

In addition, since both the forward primer and the reverse primer contain the first universal sequence, during the PCR reaction, the primer dimers formed due to the non-specific amplification of the forward primer and the reverse primer will produce a single-stranded nucleic acid with reverse sequences complementary to each other contained at its 5′ end and 3′ end, and the single-stranded nucleic acid is easy to self-anneal during the annealing stage to form a stable panhandle structure, which prevents the first universal primer and the second universal primer from performing annealing and extension of the single-stranded nucleic acid, thereby inhibiting the further amplification of primer dimers. Therefore, in the method of the present invention, the non-specific amplification of primer dimers can be effectively suppressed.

In certain embodiments, “substantially free of donor-derived nucleic acids” means not containing donor-derived nucleic acids, or, the donor-derived nucleic acid accounts for no more than 10% (e.g., no more than 5%, not more than 3%, not more than 1%, or lower) of the total nucleic acids in the fifth sample.

In certain embodiments, the fifth sample is from the recipient (e.g., the recipient who has or has not undergone transplantation); for example, the fifth sample comprises a cell or tissue from the recipient; for example, the fifth sample is selected from skin, saliva, urine, blood, hair, nails, or any combination thereof from the recipient.

In some embodiments, in step (a), for each candidate SNP site, a detection probe is also provided, the detection probe comprises a nucleotide sequence specific to the target nucleic acid and is capable of annealing or hybridizing to a region containing the candidate SNP site in the target nucleic acid, and the detection probe is labeled with a reporter group and a quencher group, wherein the reporter group can emit a signal, and the quencher group is capable of absorbing or quenching the signal emitted by the reporter group; and, the signal emitted by the detection probe when hybridized to its complementary sequence is different from the signal sent when it is not hybridized to its complementary sequence;

Furthermore, in step (c), the detection probes are used to perform melting curve analysis on the amplification products corresponding to the fifth sample obtained in step (b).

In certain embodiments, the fifth sample comprises DNA (e.g., genomic DNA).

In a fourth aspect, the present application provides a method for detecting the presence or proportion of nucleic acids of a donor in a sample from a recipient who has undergone transplantation, wherein the method comprises the following steps:
(1) providing a nucleic acid-containing sample to be tested from the recipient who has been transplanted with a cell, tissue or organ form the donor;
(2) identifying a plurality (e.g., at least 5, at least 6, at least 7, at least 8, at least 9, at least 10 or more) of such candidate SNP sites, in which the candidate SNP sites exhibit at least a first allele and a second allele in the species to which the recipient belongs, and, at the candidate SNP site, the recipient has a first genotype comprising a homozygous first allele;
(3) performing quantitative detection of each allele of each candidate SNP site in the sample to be tested;
(4) according to the quantitative detection results of step (3), selecting such a target SNP site from the candidate SNP sites, at which the sample to be tested exhibits a signal of the first allele and a signal of the second allele;
(5) according to the results of quantitative detection of the first allele and the second allele of the target SNP site, determining the presence or proportion of the nucleic acids from the donor in the sample to be tested.

In certain embodiments, in step (2), different alleles at an SNP site can be discriminated by a mechanism selected from the following so as to identify the candidate SNP site: probe hybridization, primer extension, hybridization ligation and specific digestion. In certain embodiments, in step (2), the candidate SNP site can be identified by a method selected from the group consisting of sequencing method (e.g., first-generation sequencing, pyrosequencing, next-generation sequencing), chip method (e.g., using solid-phase chip or liquid-phase chip capable of detecting SNP), qPCR-based detection method (e.g., Tagman probe method), mass spectrometry (e.g., iPLEX™ Gold based on MassARRAY), chromatography (e.g., denaturing high performance liquid chromatography (dHPLC)), electrophoresis (e.g., SNPshot method), detection method based on melting curve analysis. In certain embodiments, in step (2), the candidate SNP site is identified by a detection method based on multiplex PCR combined with melting curve analysis.

In certain embodiments, the candidate SNP site is identified by the method as previously described.

In some embodiments, in step (3), each allele of each candidate SNP site is quantitatively detected by digital PCR.

In certain embodiments, step (3) is performed by the following scheme:
(I) selecting a plurality (e.g., at least 5, at least 6, at least 7, at least 8, at least 9, at least 10 or more) of candidate SNP sites from step (2), and, for each selected candidate SNP site, providing an amplification primer set and a probe set, wherein,
(I-1) the amplification primer set comprises at least one amplification primer (e.g., a pair of amplification primers or more amplification primers), which can specifically amplify the nucleic acid containing the candidate SNP site under a condition that allows acid hybridization or annealing;
(I-2) the probe set comprises a first probe and a second probe; wherein,
(i) the first probe and the second probe are each independently labeled with a reporter group and a quencher group, wherein the reporter group can emit a signal, and the quencher group is capable of absorbing or quenching the signal emitted by the reporter group; and, the first probe and the second probe are respectively labeled with different reporter groups (e.g., fluorophores); and
(ii) the first probe is capable of hybridizing or annealing (preferably completely complementary) to a nucleic acid molecule containing the first allele of the candidate SNP site, and the second probe is capable of hybridizing or annealing (preferably completely complementary) to a nucleic acid molecule containing the second allele of the candidate SNP site; and, the first probe and the second probe are specific for different alleles;
(II) performing digital PCR on the sample to be tested from the recipient by using the amplification primer set and the probe set to quantitatively detect the nucleic acid molecule having the first allele and the nucleic acid molecule having the second allele;

In certain embodiments, the first probe specifically anneals or hybridizes to the nucleic acid molecule having the first allele during the digital PCR reaction; and the second probe specifically anneals or hybridizes to the nucleic acid molecule having the second allele during the digital PCR reaction;

In certain embodiments, the first probe does not anneal or hybridize to the nucleic acid molecule having the second allele during the digital PCR reaction; and/or, the second probe does not anneal or hybridize to the nucleic acid molecule having the first allele during the digital PCR reaction;

In the method of the present application, taking the first probe in the probe set as an example, it can hybridize or anneal (preferably completely complementary) to the nucleic acid molecule having the first allele. Therefore, when performing the digital PCR reaction, during annealing or extension process, the first probe will form a duplex with the nucleic acid molecule and be degraded by a nucleic acid polymerase (e.g., DNA polymerase) during amplification, releasing a reporter group (e.g., a fluorophore). Thus, after the digital PCR amplification reaction is completed, the endpoint fluorescence of each droplet is detected by a droplet detector, and according to the intensity of signal (e.g., a first fluorescence signal) of the free first reporter group (e.g., a first fluorophore), the numbers of positive droplets and negative droplets can be determined, thereby determining the amount of nucleic acid molecules having the first allele in the sample. Similarly, after the digital PCR amplification reaction is completed, the endpoint fluorescence of each droplet is detected by a droplet detector, and according to the intensity of signal (e.g., a second fluorescence signal) of the free second reporter group (e.g., a second fluorophore), the numbers of positive droplets and negative droplets can be determined, and the amount of nucleic acid molecules having the second allele in the sample can be determined. Since the genotypes of the donor/recipient are different, the contents corresponding to the first/second alleles are different. Therefore, by comparing and analyzing the amounts of nucleic acid molecules containing the first/second alleles, the presence or absence of a donor in the sample from the recipient can be determined, and optionally, the ratio of donor can be determined.

In the method of the present application, in some embodiments, the first probe does not anneal or hybridize to the nucleic acid molecule having the second allele during the digital PCR reaction; and/or, the second probe does not anneal or hybridize to the nucleic acid molecule having the first allele during the digital PCR reaction. It is easy to understand that the hybridization specificity of the first/second probe is particularly advantageous, which can help to accurately determine the content of the first allele/second allele, thereby helping to calculate the ratios of the donor sample and the recipient sample, respectively. In some embodiments, the hybridization specificity of the first/second probe can be obtained by controlling the annealing temperature and/or the extension temperature of the digital PCR reaction. For example, the annealing temperature and/or extension temperature can be set to be lower than the melting point of the duplex formed by the first probe and the nucleic acid molecule having the first allele, but higher than the melting point of the duplex formed by the first probe and the nucleic acid molecule having the second allele, such that the first probe hybridizes to the nucleic acid molecule having the first allele but not to the nucleic acid molecule having the second allele during the digital PCR reaction. Similarly, the annealing temperature and/or extension temperature can be set to be lower than the melting point of the duplex formed by the second probe and the nucleic acid molecule having the second allele, but higher than the melting point of the duplex formed by the second probe and the nucleic acid molecule having the first allele, such that the second probe hybridizes to the nucleic acid molecule having the second allele but not to the nucleic acid molecule having the first allele during the digital PCR reaction.

In the method of the present application, the copy number of allele can be detected by the digital PCR platform and directly output by the software according to the Poisson distribution principle. The relevant principles and calculation methods can be found in, for example, Milbury C A, Zhong Q, Lin J, et al. Determining lower limits of detection of digital PCR assays for cancer to related gene mutations. Biomol Detect Quantif 2014; 1(1):8 to 22. Published 2014 August 20. doi:10.1016/j.bdq.2014.08.001.

In some embodiments, in step (5), the quantitative detection results of the second allele of the plurality (for example at least 5, at least 6, at least 7, at least 8, at least 9, at least 10 or more) of target SNP sites are subjected to cluster analysis; then, according to the result of the cluster analysis, the genotype of the donor at each target SNP site is determined; then, according to the genotypes of the recipient and the donor at each target SNP site, as well as the quantitative detection results of the first allele and the second allele in the sample to be tested, the presence or proportion of the nucleic acids from the donor in the sample to be tested is determined.

Since the recipient contains a homozygous first allele at the target SNP site, the signal of the second allele detected in the sample to be tested must originate from the donor. In other words, the genotype of the donor at the target SNP site may be homozygous second allele, or heterozygous first and second alleles. Theoretically, for the same one sample, during the digital PCR quantitative detection process, the detection result (corresponding to the absolute copy number) exhibited by the second allele of the homozygous SNP site will be twice the detection result exhibited by the second allele of the heterozygous SNP site. Therefore, by performing cluster analysis on the detection results of the second allele of the plurality of target SNP sites, it can be determined that the donor has homozygous second allele at the target SNP site, and that the donor has heterozygous first and second alleles at the target SNP site, in which the detection result (corresponding to the absolute copy number) of the former will be twice that of the latter. In other words, by performing cluster analysis on the detection signals of the second allele, the genotype of the donor at each target SNP site can be determined. On this basis, according to the genotypes of the recipient and the donor at each target SNP site, and the quantitative detection results of the first allele and the second allele in the sample to be tested, the presence or proportion of the donor's nucleic acids in the sample to be tested can be easily determined.

In certain embodiments, before step (3), the sample to be tested from the recipient undergoes a pretreatment.

In certain embodiments, the pretreatment comprises extracting nucleic acids from the sample and/or enriching nucleic acids in the sample (e.g., by concentration and/or amplification).

In certain embodiments, wherein the recipient has received or transplanted with a hematopoietic stem cell (e.g., bone marrow hematopoietic stem cell, peripheral blood hematopoietic stem cell, umbilical cord blood hematopoietic stem cell or any combination thereof) or a tissue or organ (e.g., spinal cord) containing a hematopoietic stem cell from the donor.

In certain embodiments, the sample to be tested comprises blood (e.g., peripheral blood) or component thereof (e.g., blood cell, plasma, monocyte, granulocyte, T cell, or any combination) from the recipient after transplantation.

In certain embodiments, wherein the recipient has received or transplanted with an organ (e.g., kidney, heart, lung, liver, pancreas, or any combination thereof) from the donor.

In certain embodiments, the recipient has received or transplanted with a kidney from the donor.

In certain embodiments, the sample to be tested comprises blood (e.g., peripheral blood) or urine (especially in the case of kidney transplantation) from the recipient after transplantation.

In certain embodiments, wherein steps (a) to (b) of the method are performed by a scheme comprising steps (I) to (VI):
(I) providing the fifth sample, the first universal primer and the second universal primer, and the target-specific primer pair; and optionally, the detection probe;
(II) mixing the fifth sample with the first universal primer, the second universal primer, the target-specific primer pair, a nucleic acid polymerase, and optionally, the detection probe;
(III) incubating the product of the previous step under a condition that allows nucleic acid denaturation;
(IV) incubating the product of the previous step under a condition that allows nucleic acid annealing or hybridization;
(V) incubating the product of the previous step under a condition that allows nucleic acid extension; and
(VI) optionally, repeating steps (III) to (V) once or more times.

In certain embodiments, in step (III), the product of step (II) is incubated at a temperature of 80 to 105° C., thereby performing the nucleic acid denaturation.

In certain embodiments, in step (III), the product of step (II) is incubated for 10 to 20 s, 20 to 40 s, 40 to 60 s, 1 to 2 min, or 2 to 5 min.

In certain embodiments, in step (IV), the product of step (III) is incubated at a temperature of 35 to 40° C., 40 to 45° C., 45 to 50° C., 50 to 55° C., 55 to 60° C., 60 to 65° C., or 65 to 70° C., thereby allowing the nucleic acid annealing or hybridization.

In certain embodiments, in step (IV), the product of step (III) is incubated for 10 to 20 s, 20 to 40 s, 40 to 60 s, 1 to 2 min, or 2 to 5 min.

In certain embodiments, in step (V), the product of step (IV) is incubated at a temperature of 35 to 40° C., 40 to 45° C., 45 to 50° C., 50 to 55° C., 55 to 60° C., 60 to 65° C., 65 to 70° C., 70 to 75° C., 75 to 80° C., 80 to 85° C., thereby allowing the nucleic acid extension.

In certain embodiments, in step (V), the product of step (IV) is incubated for 10 to 20 s, 20 to 40 s, 40 to 60 s, 1 to 2 min, 2 to 5 min, 5 to 10 min, 10 to 20 min or 20 to 30 min.

In certain embodiments, steps (IV) and (V) are performed at the same or different temperatures.

In certain embodiments, steps (III) to (V) are repeated at least once, for example, at least 2 times, at least 5 times, at least 10 times, at least 20 times, at least 30 times, at least 40 times, or at least 50 times. In certain embodiments, when steps (III) to (V) are repeated once or more times, the conditions used for each cycle of steps (III) to (V) are each independently the same or different.

In certain embodiments, the primers of the amplification primer set each independently have a length of 15 to 20 nt, 20 to 30 nt, 30 to 40 nt, 40 to 50 nt, 50 to 60 nt, 60 to 70 nt, 70 nt to 80 nt, 80 to 90 nt, 90 to 100 nt, 100 to 110 nt, 110 to 120 nt, 120 to 130 nt, 130 to 140 nt, 140 to 150 nt.

In certain embodiments, the primers of the amplification primer set, or any components thereof, each independently comprise or consist of naturally occurring nucleotides (e.g., deoxyribonucleotides or ribonucleotides), modified nucleotides, non-natural nucleotides, or any combination thereof.

In certain embodiments, the amplification primer set each independently comprises a primer pair having nucleotide sequences selected from the following or any combination thereof (e.g., any 5 pairs, 10 pairs, 15 pairs, 20 pairs, 23 pairs): SEQ ID NOs: 72 and 73; 77 and 76; 80 and 81; 84 and 85; 88 and 89; 92 and 93; 96 and 97; 100 and 101; 104 and 105; 108 and 109; and 113; 116 and 117; 120 and 121; 124 and 125; 128 and 129; 132 and 133; 136 and 137; 140 and 141; 144 and 145; 148 and 149; 152 and 153; 156 and 157; 160 and 161.

In some embodiments, the first probe and the second probe each independently comprise or consist of naturally occurring nucleotides (e.g., deoxyribonucleotides or ribonucleotides), modified nucleotides, non-natural nucleotides (e.g., peptide nucleic acid (PNA) or locked nucleic acid), or any combination thereof.

In certain embodiments, the first probe and the second probe each independently have a length of 15 to 20 nt, 20 to 30 nt, 30 to 40 nt, 40 to 50 nt, 50 to 60 nt, 60 nt to 70 nt, 70 to 80 nt, 80 to 90 nt, 90 to 100 nt, 100 to 200 nt, 200 to 300 nt, 300 to 400 nt, 400 to 500 nt, 500 to 600 nt, 600 to 700 nt, 700 nt to 800 nt, 800 to 900 nt, 900 to 1000 nt.

In certain embodiments, the first probe and the second probe each independently have a 3-OH end; alternatively, the 3′-end of the probe is blocked; for example, the 3′-end of the probe is blocked by adding a chemical moiety (e.g., biotin or alkyl) to the 3-OH of the last nucleotide of the probe, or by removing the 3-OH of the last nucleotide of the probe, or by replacing the last nucleotide with a dideoxynucleotide.

In certain embodiments, the first probe and the second probe each independently are a self-quenching probe; for example, the probe is labeled with a reporter group at or upstream of its 5′ end and labeled with a quencher group at or downstream of its 3′ end, or labeled with a reporter group at or downstream of its 3′ end and labeled with a quencher group at or upstream of its 5′ end. In certain embodiments, the reporter group and quencher group are separated by a distance of 10 to 80 nt or longer.

In certain embodiments, the reporter groups in the probes are each independently a fluorophore (e.g., ALEX-350, FAM, VIC, TET, CAL Fluor Gold 540, JOE, HEX, CAL Fluor Orange 560, TAMRA, CAL Fluor Red 590, ROX, CAL Fluor Red 610, TEXAS RED, CAL Fluor Red 635, Quasar 670, CY3, CY5, CY5.5, Quasar 705); and the quencher group is a molecule or group (e.g., DABCYL, BHQ (e.g., BHQ-1 or BHQ-2), ECLIPSE, and/or TAMRA) capable of absorbing/quenching the fluorescence.

In certain embodiments, the first probe and the second probe are each independently linear, or have a hairpin structure.

In certain embodiments, the first probe and the second probe have different reporter groups. In certain embodiments, the first probe and the second probe are degradable by a nucleic acid polymerase (e.g., a DNA polymerase).

In certain embodiments, the probe set comprises probes having nucleotide sequences selected from the following or any combination thereof (e.g., any combination of 5, 10, 20, 40, 60): SEQ ID NOs: 73, 74, 78, 79, 82, 83, 86, 87, 90, 91, 94, 95, 98, 99, 102, 103, 106, 107, 110, 111, 114, 115, 118, 119, 122, 123, 126, 127, 130, 131, 134, 135, 138, 139, 142, 143, 146, 147, 150, 151, 154, 155, 158, 159, 162, 163.

In a fifth aspect, the present application provides a method for detecting an SNP site with different genotypes between a donor and a recipient, comprising the following steps:
(a) providing a third sample from the recipient and a fourth sample from the recipient after undergoing transplantation, wherein the third sample contains one or more target nucleic acids derived from the recipient, and substantially does not contain nucleic acids derived from the donor; the fourth sample contains one or more target nucleic acids derived from the donor, and the target nucleic acid comprises one or more candidate SNP sites, and,
providing a first universal primer and a second universal primer, and, for each candidate SNP site, providing at least one target-specific primer pair; wherein,
the first universal primer comprises a first universal sequence;
the second universal primer comprises a second universal sequence, the second universal sequence comprises the first universal sequence and additionally comprises at least one nucleotide at the 3′ end of the first universal sequence;
the target-specific primer pair is capable of performing amplification using the target nucleic acid as a template to generate a nucleic acid product containing the candidate SNP site, and the target-specific primer pair comprises a forward primer and a reverse primer, wherein, the forward primer comprises the first universal sequence and a forward nucleotide sequence specific to the target nucleic acid, and the forward nucleotide sequence is located at the 3′ end of the first universal sequence; the reverse primer comprises the second universal sequence and a reverse nucleotide sequence specific to the target nucleic acid, and the reverse nucleotide sequence is located at the 3′ end of the second universal sequence; and, the second universal sequence is not completely complementary to the complementary sequence of the forward primer; and
(b) under a condition that allows nucleic acid amplification, amplifying the target nucleic acids in the third sample and the fourth sample by using the first universal primer and the second universal primer and the target-specific primer pair, respectively, thereby obtaining amplification products corresponding to the third sample and the fourth sample respectively;
(c) performing melting curve analysis on the amplification products corresponding to the third sample and the fourth sample obtained in step (b);
(d) according to the result of the melting curve analysis of step (c), determining such a SNP site at which the third sample only exhibits a first allele, and the fourth sample exhibits at least a second allele (e.g., exhibiting the first and second alleles); in which the SNP site is an SNP site with different genotypes between the donor and the recipient;

In the method of the present application, the forward primer and the reverse primer respectively comprise a forward nucleotide sequence and a reverse nucleotide sequence specific to the target nucleic acid, thus, during the PCR reaction, the target-specific primer pair (forward primer and reverse primer) will anneal to the target nucleic acid and initiate PCR amplification to produce an initial amplification product comprising two nucleic acid strands (nucleic acid strand A and nucleic acid strand B). Further, since both the forward primer and the first universal primer contain the first universal sequence, the nucleic acid strand A complementary to the forward primer can also be complementary to the first universal primer. Similarly, the nucleic acid strand B complementary to the reverse primer can also be complementary to the second universal primer.

Therefore, as the PCR reaction proceeds, the first universal primer and the second universal primer will respectively anneal to the nucleic acid strand A and the nucleic acid strand B of the initial amplification product, and further initiate PCR amplification. In this process, since the reverse primer/second universal primer contains the first universal sequence, the first universal primer not only can anneal to the nucleic acid strand A (the nucleic acid strand complementary to the forward primer/first universal primer) and synthesize its complementary strand, but also can anneal to the nucleic acid strand B (the nucleic acid strand complementary to the reverse primer/second universal primer) and synthesize its complementary strand. That is, the first universal primer can simultaneously amplify the nucleic acid strand A and the nucleic acid strand B of the initial amplification product. At the same time, the second universal primer contains an additional nucleotide at the 3′ end of the first universal primer, although the second universal primer may also anneal to the nucleic acid strand A (the nucleic acid strand complementary to the forward primer/first universal primer, which has a sequence complementary to the forward primer), but it does not match the nucleic acid strand A at the 3′ end (i.e., not completely complementary at the 3′ end). Thus, during the amplification process, the second universal primer will preferentially anneal to the nucleic acid strand B (the nucleic acid strand complementary to the reverse primer/second universal primer) and synthesize its complementary strand, while substantially unable to perform extension to synthesize the complementary strand of the nucleic acid strand A (the nucleic acid strand complementary to first forward primer/first universal primer).

Therefore, as the PCR amplification proceeds, the synthesis efficiency of the complementary strand (nucleic acid strand B) of the nucleic acid strand A will be significantly lower than that of the complementary strand (nucleic acid strand A) of the nucleic acid strand B, so that the complementary strand (nucleic acid strand A) of the nucleic acid strand B is synthesized and amplified in large quantities, while the synthesis and amplification of the complementary strand (nucleic acid strand B) of the nucleic acid strand A is inhibited, resulting in a large amount of a single-stranded product (nucleic acid strand A, which contains the sequence complementary to the forward primer/first universal primer and the sequence of the reverse primer/second universal primer), and realizing the asymmetric amplification of the target nucleic acid containing one or more SNP sites. Thus, in steps (a) and (b) of the method of the present application, the asymmetric amplification of one or more target nucleic acids in the sample is achieved.

In addition, since both the forward primer and the reverse primer contain the first universal sequence, during the PCR reaction, the primer dimers formed due to the non-specific amplification of the forward primer and the reverse primer will produce a single-stranded nucleic acid with reverse sequences complementary to each other contained at its 5′ end and 3′ end, and the single-stranded nucleic acid is easy to self-anneal during the annealing stage to form a stable panhandle structure, which prevents the first universal primer and the second universal primer from performing annealing and extension of the single-stranded nucleic acid, thereby inhibiting further amplification of the primer dimers. Therefore, in the method of the present invention, non-specific amplification of primer dimers can be effectively suppressed.

In certain embodiments, in step (d) of the method, the genotypes of each candidate SNP site in the third sample and the fourth sample are determined according to the result of the melting curve analysis, thereby determining such an SNP site at which the third sample only exhibits a first allele, and the fourth sample only exhibits the first and second alleles;

In certain embodiments, “substantially free of nucleic acids from the donor” means that there is free of nucleic acids from the donor, or, the nucleic acids from the donor accounts for no more than 10% (e.g., no more than 5%, not more than 3%, not more than 1%, or lower) of the total nucleic acids in the second sample.

In certain embodiments, the third sample is from the recipient (e.g., the recipient who has or has not undergone transplantation); for example, the third sample comprises a cell or tissue from the recipient; for example, the third sample is selected from skin, saliva, urine, blood, hair, nail, or any combination thereof from the recipient;

In certain embodiments, for the recipient who has not undergone transplantation, the third sample can be any cell or tissue (e.g., skin, saliva, urine, blood, etc.). For the recipient who has undergone transplantation, the third sample contains substantially no nucleic acids from the donor.

In some preferred embodiments, for the recipient who has undergone hematopoietic stem cell transplantation, the third sample can be selected from skin, saliva, urine, hair, nail, or tissue, etc., but cannot be selected from blood, because the blood sample from the recipient who has undergone hematopoietic stem cell transplantation may contain a large amount of the nucleic acids from the donor. In some preferred embodiments, for the recipient who has undergone kidney transplantation, the third sample can be selected from skin, saliva, hair, nail, or tissue, etc., but cannot be selected from blood and urine, because the blood and urine samples from the recipient who has undergone kidney transplantation may contain a large amount of the nucleic acids from the donor. In some preferred embodiments, for the recipient who has undergone liver transplantation, the third sample can be selected from skin, saliva, hair, nails, urine, or tissue, etc., but cannot be selected from blood, because the blood sample from the recipient who has undergone kidney transplantation may contain a large amount of the nucleic acids from the donor.

In certain embodiments, in the fourth sample, the amount of the nucleic acids from the donor accounts for at least 20%, such as at least 25%, at least 30%, at least 35%, at least 40%, at least 50%, or higher of the amount of total nucleic acids in the fourth sample;

In certain embodiments, the recipient has received or transplanted with an organ, tissue or cell from the donor;

For example, the recipient has received or transplanted with an organ (e.g., kidney, heart, lung, liver, pancreas, or any combination thereof) from the donor; in certain embodiments, the fourth sample comprises the blood (e.g., peripheral blood) or urine (especially in the case of kidney transplantation) from the recipient who has undergone transplantation; in certain embodiments, the fourth sample comprises the blood (e.g., peripheral blood) or urine (especially in the case of kidney transplantation) from the recipient who has undergone transplantation no more than 5 days (e.g., no more than 3 days, 2 days or 1 day);

For example, the recipient has received or transplanted with a hematopoietic stem cell (e.g., bone marrow hematopoietic stem cell, peripheral blood hematopoietic stem cell, umbilical cord blood hematopoietic stem cell) or a hematopoietic stem cell-containing tissue or organ (e.g., bone marrow) from the donor; in certain embodiments, the fourth sample comprises the blood (e.g., peripheral blood) or component thereof (e.g., blood cell) from the recipient who has undergone transplantation; in certain embodiments, the fourth sample comprises the blood (e.g., peripheral blood) or component thereof (e.g., blood cell) from the recipient who has undergone transplantation at least 5 days (e.g., at least 10 days, at least 15 days, at least 20 days, at least 30 days);

In some embodiments, in step (a), for each candidate SNP site, a detection probe is also provided, the detection probe comprises a nucleotide sequence specific to the target nucleic acid and is capable of performing annealing or hybridization to a region containing the candidate SNP site in the target nucleic acid, and the detection probe is labeled with a reporter group and a quencher group, wherein the reporter group can send a signal, and the quencher group is capable of absorbing or quenching the signal emit by the reporter group; and, the signal emitted by the detection probe when hybridized to its complementary sequence is different from the signal sent when it is not hybridized to its complementary sequence;

And, in step (c), the detection probe is used to perform melting curve analysis on the amplification products corresponding to the third sample and the fourth sample obtained in step (b), respectively;

In certain embodiments, the third sample comprises DNA (e.g., genomic DNA).

In certain embodiments, the fourth sample comprises DNA (e.g., genomic DNA).

In a sixth aspect, the present application provides a method for detecting the presence or proportion of nucleic acids from a donor in a sample from a recipient who has undergone transplantation, wherein the method comprises the following steps:
(1) providing a nucleic acid-containing sample to be tested from the recipient who has been transplanted with a cell, tissue or organ from the donor;
(2) identifying a plurality (e.g., at least 5, at least 6, at least 7, at least 8, at least 9, at least 10 or more) of target SNP sites, wherein, at the target SNP sites, the recipient has a first genotype comprising a homozygous first allele, and the donor has a second genotype comprising a second allele, wherein the first genotype is different from the second genotype, and the first allele is different from the second allele;
(3) performing quantitative detection on the first allele and the second allele of each target SNP site in the sample to be tested;
(4) according to the results of quantitative detection of the first allele and the second allele of the target SNP site, determining the presence or proportion of the nucleic acids from the donor in the sample to be tested;

In some embodiments, in step (2), the target SNP site can be identified by discriminating different alleles at an SNP site by a mechanism selected from the following: probe hybridization, primer extension, hybridization ligation and specific digestion. In some embodiments, in step (2), the target SNP site can be identified by a method selected from the following: sequencing method (e.g., first-generation sequencing method, pyrosequencing method, second-generation sequencing method), chip method (e.g., using solid-phase chip or liquid-phase chip capable of detecting SNP), qPCR-based detection method (e.g., Tagman probe method), mass spectrometry (e.g., iPLEX™ Gold based on MassARRAY), chromatography (e.g., denaturing high performance liquid chromatography (dHPLC), electrophoresis (e.g., SNPshot method), detection method based on melting curve analysis. In certain embodiments, in step (2), the target SNP site is identified by a detection method based on multiplex PCR combined with melting curve analysis.

In certain embodiments, the target SNP site is identified by the method as previously described.

In some embodiments, in step (3), the first allele and the second allele of each target SNP site in the sample are quantitatively detected by digital PCR, respectively.

In certain embodiments, step (3) is performed by the following scheme:
(I) for each target SNP site, providing an amplification primer set and a probe set, wherein,
(I-1) the amplification primer set comprises at least one amplification primer (e.g., a pair of amplification primers or more amplification primers), which can specifically amplify the nucleic acid molecule containing the target SNP site under a condition that allows acid hybridization or annealing;
(I-2) the probe set comprises a first probe and a second probe; wherein,
(i) the first probe and the second probe are each independently labeled with a reporter group and a quencher group, wherein the reporter group can emit a signal, and the quencher group is capable of absorbing or quenching the signal emitted by the reporter group; and, the first probe and the second probe are respectively labeled with different reporter groups (e.g., fluorophores); and
(ii) the first probe is capable of hybridizing or annealing (preferably completely complementary) to a nucleic acid molecule containing the first allele of the target SNP site, and the second probe is capable of hybridizing or annealing (preferably completely complementary) to a nucleic acid molecule containing the second allele of the target SNP site; and, the first probe and the second probe are specific for different alleles;
(II) using the amplification primer set and the probe set to perform digital PCR on the sample to be tested, and performing quantitative detection on the nucleic acid molecule having the first allele and the nucleic acid molecule having the second allele.

In certain embodiments, the first probe specifically anneals or hybridizes to the nucleic acid molecule having the first allele during the digital PCR reaction; and the second probe specifically anneals or hybridizes to the nucleic acid molecule having the second allele during the digital PCR reaction.

In certain embodiments, the first probe does not anneal or hybridize to the nucleic acid molecule having the second allele during the digital PCR reaction; and/or, the second probe does not anneal or hybridize to the nucleic acid molecule having the first allele during the digital PCR reaction.

In the method of the present application, taking the first probe in the probe set as an example, it can hybridize or anneal (preferably completely complementary) to the nucleic acid molecule having the first allele. Therefore, when performing the digital PCR reaction, during the annealing or extension process, the first probe will form a duplex with the nucleic acid molecule and be degraded by a nucleic acid polymerase (e.g., DNA polymerase) during amplification to release a reporter group (e.g., a fluorophore). Thus, after the digital PCR amplification reaction is completed, the endpoint fluorescence of each droplet is detected by a droplet detector, and according to the signal (e.g., a first fluorescence signal) of the free first reporter group (e.g., a first fluorophore), the numbers of positive droplets and negative droplets can be determined, and the amount of the nucleic acid molecule having the first allele in the sample can be determined. Similarly, after the digital PCR amplification reaction is completed, the endpoint fluorescence of each droplet is detected by a droplet detector, and according to the signal (e.g., a second fluorophore) of the free second reporter group (e.g., a second fluorophore), the numbers of positive droplets and negative droplets can be determined, and the amount of the nucleic acid molecule having the second allele in the sample can be determined. Since the genotypes of the donor/recipient are different, the contents corresponding to the first/second alleles are different. Therefore, by comparing and analyzing the amounts of the nucleic acid molecules containing the first/second alleles, the presence or absence of the donor in the sample from the recipient can be determined, and optionally, the ratio of the donor can be determined.

In the method of the present application, in some embodiments, the first probe does not anneal or hybridize to the nucleic acid molecule having the second allele during the digital PCR reaction; and/or, the second probe does not anneal or hybridize to the nucleic acid molecule having the first allele during the digital PCR reaction. It is easy to understand that the hybridization specificity of the first/second probe is particularly advantageous, which can help to accurately determine the content of the first allele/second allele, thereby helping to calculate the ratio thereof in each of the donor sample and the recipient sample. In some embodiments, the hybridization specificity of the first/second probe can be obtained by controlling the annealing temperature and/or the extension temperature of the digital PCR reaction. For example, the annealing temperature and/or extension temperature can be set to be lower than the melting point of the duplex formed by the first probe and the nucleic acid molecule having the first allele, but higher than the melting point of the duplex formed by the first probe and the nucleic acid molecule having the second allele, such that the first probe hybridizes to the nucleic acid molecule having the first allele but not to the nucleic acid molecule having the second allele during the digital PCR reaction. Similarly, the annealing temperature and/or extension temperature can be set to be lower than the melting point of the duplex formed by the second probe and the nucleic acid molecule having the second allele, but higher than the melting point of the duplex formed by the second probe and the nucleic acid molecule having the first allele, such that the second probe hybridizes to the nucleic acid molecule having the second allele but not to the nucleic acid molecule having the first allele during the digital PCR reaction.

In the method of the present application, the copy number of the allele can be detected by the digital PCR platform and directly output by the software according to the Poisson distribution principle. The relevant principles and calculation methods can be found in, for example, Milbury C A, Zhong Q, Lin J, et al. Determining lower limits of detection of digital PCR assays for cancer to related gene mutations. Biomol Detect Quantif 2014; 1(1):8 to 22. Published 2014 August 20. doi:10.1016/j.bdq.2014.08.001.

In some embodiments, in step (4), the quantitative detection results of the second allele of the plurality (for example at least 5, at least 6, at least 7, at least 8, at least 9, at least 10 or more) of target SNP sites are subjected to cluster analysis; then, according to the result of the cluster analysis, the genotype of the donor at each target SNP site is determined; then, according to the genotypes of the recipient and the donor at each target SNP site, as well as the quantitative detection results of the first allele and the second allele in the sample to be tested, the presence or proportion of the nucleic acids from the donor in the sample to be tested is determined.

Since the recipient contains a homozygous first allele at the target SNP site, the signal of the second allele detected in the sample to be tested must originate from the donor. In other words, the genotype of the donor at the target SNP site may be homozygous second allele, or heterozygous first and second alleles. Theoretically, for the same one sample, during the digital PCR quantitative detection process, the detection result (corresponding to the absolute copy number) exhibited by the second allele of the homozygous SNP site will be twice the detection result exhibited by the second allele of the heterozygous SNP site. Therefore, by performing cluster analysis on the detection results of the second allele of the plurality of target SNP sites, it can be determined that the donor has homozygous second allele at the target SNP site, and that the donor has heterozygous first and second alleles at the target SNP site, in which the detection result (corresponding to the absolute copy number) of the former will be twice that of the latter. In other words, by performing cluster analysis on the detection signals of the second allele, the genotype of the donor at each target SNP site can be determined. On this basis, according to the genotypes of the recipient and the donor at each target SNP site, and the quantitative detection results of the first allele and the second allele in the sample to be tested, the presence or proportion of the donor's nucleic acids in the sample to be tested can be easily determined.

In some embodiments, before step (3), the sample to be tested from the recipient is subjected to a pretreatment.

In certain embodiments, the pretreatment comprises extracting nucleic acids from the sample and/or enriching nucleic acids in the sample (e.g., by concentration and/or amplification).

In certain embodiments, wherein the recipient has received or transplanted with a hematopoietic stem cell (e.g., bone marrow hematopoietic stem cell, peripheral blood hematopoietic stem cell, umbilical cord blood hematopoietic stem cell or any combination thereof) or a hematopoietic stem cell-containing tissue or organ (e.g., spinal marrow) from the donor;

In certain embodiments, the sample to be tested comprises the blood (e.g., peripheral blood) or component thereof (e.g., blood cell, plasma, monocyte, granulocyte, T cell, or any combination) from the recipient after transplantation.

In certain embodiments, wherein the recipient has received or transplanted with an organ (e.g., kidney, heart, lung, liver, pancreas, or any combination thereof) from the donor.

In certain embodiments, the recipient has received or transplanted with a kidney from the donor.

In certain embodiments, the sample to be tested comprises the blood (e.g., peripheral blood) or urine (especially in the case of kidney transplantation) from the recipient after transplantation.

In certain embodiments, wherein steps (a) to (b) of the method are performed by a scheme comprising steps (I) to (VI):
(I) providing the third sample and the fourth sample, the first universal primer and the second universal primer, and the target-specific primer pair; and optionally, the detection probe;
(II) mixing the sample with the first universal primer, the second universal primer, the target-specific primer pair, a nucleic acid polymerase, and optionally, the detection probe;
(III) incubating the product of the previous step under a condition that allows nucleic acid denaturation;
(IV) incubating the product of the previous step under a condition that allows nucleic acid annealing or hybridization;
(V) incubating the product of the previous step under a condition that allows nucleic acid extension; and
(VI) optionally, repeating steps (III) to (V) once or more times.

In certain embodiments, in step (III), the product of step (II) is incubated at a temperature of 80 to 105° C., thereby performing the nucleic acid denaturation.

In certain embodiments, in step (III), the product of step (II) is incubated for 10 to 20 s, 20 to 40 s, 40 to 60 s, 1 to 2 min, or 2 to 5 min.

In certain embodiments, in step (IV), the product of step (III) is incubated at a temperature of 35 to 40° C., 40 to 45° C., 45 to 50° C., 50 to 55° C., 55 to 60° C., 60 to 65° C., or 65 to 70° C., thereby allowing the nucleic acid annealing or hybridization.

In certain embodiments, in step (IV), the product of step (III) is incubated for 10 to 20 s, 20 to 40 s, 40 to 60 s, 1 to 2 min, or 2 to 5 min.

In certain embodiments, in step (V), the product of step (IV) is incubated at a temperature of 35 to 40° C., 40 to 45° C., 45 to 50° C., 50 to 55° C., 55 to 60° C., 60 to 65° C., 65 to 70° C., 70 to 75° C., 75 to 80° C., 80 to 85° C., thereby allowing the nucleic acid extension.

In certain embodiments, in step (V), the product of step (IV) is incubated for 10 to 20 s, 20 to 40 s, 40 to 60 s, 1 to 2 min, 2 to 5 min, 5 to 10 min, 10 to 20 min or 20 to 30 min.

In certain embodiments, steps (IV) and (V) are performed at the same or different temperatures.

In certain embodiments, steps (III) to (V) are repeated at least once, for example, at least 2 times, at least 5 times, at least 10 times, at least 20 times, at least 30 times, at least 40 times, or at least 50 times. In certain embodiments, when steps (III) to (V) are repeated once or more times, the conditions used for each cycle of steps (III) to (V) are each independently the same or different.

In certain embodiments, the primers of the amplification primer set each independently have a length of 15 to 20 nt, 20 to 30 nt, 30 to 40 nt, 40 to 50 nt, 50 to 60 nt, 60 to 70 nt, 70 nt to 80 nt, 80 to 90 nt, 90 to 100 nt, 100 to 110 nt, 110 to 120 nt, 120 to 130 nt, 130 to 140 nt, 140 to 150 nt.

In certain embodiments, the primers of the amplification primer set, or any component thereof, each independently comprise or consist of naturally occurring nucleotides (e.g., deoxyribonucleotides or ribonucleotides), modified nucleotides, non-natural nucleotides, or any combination thereof.

In certain embodiments, the amplification primer set each independently comprises a primer pair having nucleotide sequences selected from the following or any combination thereof (e.g., any 5 pairs, 10 pairs, 15 pairs, 20 pairs, 23 pairs): SEQ ID NOs: 72 and 73; 77 and 76; 80 and 81; 84 and 85; 88 and 89; 92 and 93; 96 and 97; 100 and 101; 104 and 105; 108 and 109; and 113; 116 and 117; 120 and 121; 124 and 125; 128 and 129; 132 and 133; 136 and 137; 140 and 141; 144 and 145; 148 and 149; 152 and 153; 156 and 157; 160 and 161.

In some embodiments, the first probe and the second probe each independently comprise or consist of naturally occurring nucleotides (e.g., deoxyribonucleotides or ribonucleotides), modified nucleotides, non-natural nucleotides (e.g., peptide nucleic acid (PNA) or locked nucleic acid), or any combination thereof.

In certain embodiments, the first probe and the second probe each independently have a length of 15 to 20 nt, 20 to 30 nt, 30 to 40 nt, 40 to 50 nt, 50 to 60 nt, 60 nt to 70 nt, 70 to 80 nt, 80 to 90 nt, 90 to 100 nt, 100 to 200 nt, 200 to 300 nt, 300 to 400 nt, 400 to 500 nt, 500 to 600 nt, 600 to 700 nt, 700 nt to 800 nt, 800 to 900 nt, 900 to 1000 nt.

In certain embodiments, the first probe and the second probe each independently have a 3′-OH end; alternatively, the 3′-end of the probe is blocked; for example, the 3′-end of the probe is blocked by adding a chemical moiety (e.g., biotin or alkyl) to the 3′-OH of the last nucleotide of the probe, or by removing the 3′-OH of the last nucleotide of the probe, or by replacing the last nucleotide with a dideoxynucleotide.

In certain embodiments, the first probe and the second probe each independently are a self-quenching probe; for example, the probe is labeled with a reporter group at or upstream of its 5′ end and labeled with a quencher group at or downstream of its or 3′ end, or labeled with a reporter group at or downstream of its 3′ end and labeled with a quencher group at or upstream of its 5′ end. In certain embodiments, the reporter group and the quencher group are separated by a distance of 10 to 80 nt or longer.

In certain embodiments, the reporter groups in the probes are each independently a fluorophore (e.g., ALEX-350, FAM, VIC, TET, CAL Fluor Gold 540, JOE, HEX, CAL Fluor Orange 560, TAMRA, CAL Fluor Red 590, ROX, CAL Fluor Red 610, TEXAS RED, CAL Fluor Red 635, Quasar 670, CY3, CY5, CY5.5, Quasar 705); and the quencher group is a molecule or group (e.g., DABCYL, BHQ (e.g., BHQ-1 or BHQ-2), ECLIPSE, and/or TAMRA) capable of absorbing/quenching the fluorescence.

In certain embodiments, the first probe and the second probe are each independently linear, or have a hairpin structure.

In certain embodiments, the first probe and the second probe have different reporter groups. In certain embodiments, the first probe and the second probe are degradable by a nucleic acid polymerase (e.g., a DNA polymerase).

In certain embodiments, the probe set comprises probes having nucleotide sequences selected from the following or any combination thereof (e.g., any combination of 5, 10, 20, 40, 60): SEQ ID NOs: 73, 74, 78, 79, 82, 83, 86, 87, 90, 91, 94, 95, 98, 99, 102, 103, 106, 107, 110, 111, 114, 115, 118, 119, 122, 123, 126, 127, 130, 131, 134, 135, 138, 139, 142, 143, 146, 147, 150, 151, 154, 155, 158, 159, 162, 163.

In some embodiments, the candidate SNP site has one or more features selected from the following:
(1) the candidate SNP site has a Fst of less than 0.3 (e.g., less than 0.2, less than 0.1, less than 0.05, less than 0.01) between different human races;
(2) the candidate SNP site is located on different chromosomes;
(3) the candidate SNP site has an allele frequency between 0.2 and 0.8 (e.g., between 0.3 and 0.7, between 0.4 and 0.6).

In some embodiments, the candidate SNP site has one or more features selected from the following:
(1) the candidate SNP site has a Fst of less than 0.01 between different human races;
(2) the candidate SNP site is located on different chromosomes;
(3) the candidate SNP site has an allele frequency between 0.3 and 0.7.

In certain embodiments, the candidate SNP site is an SNP site with a biallelic polymorphism.

In certain embodiments, the candidate SNP site is an SNP site in the human genome; for example, the target nucleic acid comprises a human genome SNP site selected from the group consisting of: rs16363, rs1610937, rs5789826, rs1611048, rs2307533, rs112552066, rs5858210, rs2307839, rs149809066, rs66960151, rs34765837, rs68076527, rs10779650, rs4971514, rs6424243, rs12990278, rs2122080, rs98506667, rs774763, rs711725, rs2053911, rs9613776, rs7160304, and any combination of the aforementioned SNP sites (e.g., any combination of 5, 10, 15, 20, 23 of the aforementioned SNP sites).

In certain embodiments, the target nucleic acid in the sample comprises the following human genome SNP sites: rs16363, rs1610937, rs5789826, rs1611048, rs2307533, rs112552066, rs5858210, rs2307839, rs149809066, rs66960151, rs34765837, rs68076527, rs10779650, rs4971514, rs6424243, rs12990278, rs2122080, rs98506667, rs774763, rs711725, rs2053911, rs9613776 and rs7160304.

In certain embodiments, in step (b), the sample is mixed with the first universal primer, the second universal primer, the target-specific primer pair, and a nucleic acid polymerase, and subjected to nucleic acid amplification (e.g., PCR reaction), and then, the detection probe is added to the product of step (b), and melting curve analysis is carried out; or, in step (b), the sample is mixed with the first universal primer, the second universal primer, the target-specific primer pair, the detection probe, and a nucleic acid polymerase, and subjected to nucleic acid amplification (e.g., PCR reaction), and then, after the PCR reaction is completed, melting curve analysis is carried out.

In certain embodiments, the detection probe comprises or consists of naturally occurring nucleotides (e.g., deoxyribonucleotides or ribonucleotides), modified nucleotides, non-natural nucleotides (e.g., peptide nucleic acid (PNA) or locked nucleic acid), or any combination thereof. In certain preferred embodiments, the detection probe comprises modified nucleotides, such as modified deoxyribonucleotides or ribonucleotides, such as 5-methylcytosine or 5-hydroxymethylcytosine. In certain preferred embodiments, the detection probe comprises non-natural nucleotides, such as deoxyinosine, inosine, 1-(2′-deoxy-β-D-ribofuranosyl)-3-nitropyrrole, 5-nitroindole or locked nucleic acid (LNA).

In certain embodiments, the detection probe has a length of 15 to 20 nt, 20 to 30 nt, 30 to 40 nt, 40 to 50 nt, 50 to 60 nt, 60 to 70 nt, 70 to 80 nt, 80 to 90n t, 90 to 100 nt, 100 to 200 nt, 200 to 300 nt, 300 to 400 nt, 400 to 500 nt, 500 to 600 nt, 600 to 700 nt, 700 to 800 nt, 800 to 900 nt, 900 to 1000 nt.

In certain embodiments, the detection probe has a 3′-OH end; alternatively, the 3′-end of the detection probe is blocked; for example, the 3′-end of the detection probe is blocked by adding a chemical moiety (e.g., biotin or alkyl) to the 3′-OH of the last nucleotide of the detection probe, by removing the 3′-OH of the last nucleotide of the detection probe, or replacing the last nucleotide with a dideoxynucleotide.

In certain embodiments, the detection probe is a self-quenching probe; for example, the detection probe is labeled with a reporter group at or upstream of its 5′ end and labeled with a quencher group at or downstream of its 3′ end, or labeled with a reporter group at or downstream of its 3′ end and labeled with a quencher group at or upstream of its 5′ end. In such embodiments, when the detection probe is not hybridized to other sequence, the quencher group is located at a position capable of absorbing or quenching the signal of the reporter group (e.g., the quencher is located adjacent to the reporter group), thereby absorbing or quenching the signal emitted by the reporter group. In this case, the detection probe does not emit a signal. Further, when the detection probe is hybridized to its complementary sequence, the quencher group is located at a position incapable of absorbing or quenching the signal of the reporter group (e.g., the quencher group is located at a position far away from the reporter group), thereby not absorbing or quenching the signal emitted by the reporter group. In this case, the detection probe emits a signal.

The design of such self-quenching detection probes is within the capability of those skilled in the art. For example, the detection probe can be labeled with a reporter group at the 5′ end and a quencher group at the 3′ end, or the detection probe can be labeled with a reporter group at the 3′ end and a quencher group at the 5′ end. Thus, when the detection probe exists alone, the reporter group and the quencher group approach and interact with each other, so that the signal emitted by the reporter group is absorbed by the quencher group, so that the detection probe does not emit a signal; and when the detection probe hybridizes to its complementary sequence, the reporter group and the quencher group are separated from each other, so that the signal emitted by the reporter group cannot be absorbed by the quencher group, so that the detection probe emits a signal.

However, it should be understood that the reporter group and the quencher group need not necessarily be labeled at the ends of the detection probe. A reporter group and/or a quencher group can also be labeled inside the detection probe, as long as a signal emitted by the detection probe when it is hybridized to its complementary sequence is different from a signal emitted when it is not hybridized to its complementary sequence. For example, a reporter group can be labeled upstream (or downstream) of the detection probe, and a quencher group can be labeled downstream (or upstream) of the detection probe, and the two are separated by a sufficient distance (e.g., 10 to 20 nt, 20 to 30 nt, 30 to 40 nt, 40 to 50 nt, 50 to 60 nt, 60 to 70 nt, 70 to 80 nt, or longer). Thus, when the detection probe exists alone, the reporter group and the quencher group are close to and interact between each other due to the free coil of the probe molecule or the formation of secondary structure (e.g., a hairpin structure) of the probe, so that the signal sent by the reporter group is absorbed by the quencher group, and the detection probe does not emit a signal; and, when the detection probe hybridizes to its complementary sequence, the reporter group and the quencher group are separated from each other by a sufficient distance such that the signal emitted by the reporter group cannot be absorbed by the quencher group, thereby allowing the detection probe to emit a signal. In certain preferred embodiments, the reporter group and the quencher group are separated by a distance of 10 to 80 nt or longer, such as 10 to 20 nt, 20 to 30 nt, 30 to 40 nt, 40 to 50 nt, 50 nt to 60 nt, 60 to 70 nt, 70 to 80 nt. In certain preferred embodiments, the reporter group and the quencher group are separated by no more than 80 nt, no more than 70 nt, no more than 60 nt, no more than 50 nt, no more than 40 nt, no more than 30 nt, or no more than 20 nt. In certain preferred embodiments, the reporter group and the quencher group are separated by a distance of least 5 nt, at least 10 nt, at least 15 nt, or at least 20 nt.

Thus, the reporter group and the quencher group can be labeled at any suitable position on the detection probe, as long as a signal emitted by the detection probe when it hybridized to its complementary sequence is different from a signal emitted when it is not hybridized to its complementary sequence. However, in certain preferred embodiments, at least one of the reporter group and the quencher group is located at an end (e.g., 5′ or 3′ end) of the detection probe. In some preferred embodiments, one of the reporter group and the quencher group is located at the 5′ end of the detection probe or at a position 1 to 10 nt away from the 5′ end, and the reporter group and the quencher group are separated by a suitable distance so that the quencher group is able to absorb or quench the signal of the reporter group before the detection probe is hybridized to its complementary sequence. In certain preferred embodiments, one of the reporter group and the quencher group is located at the 3′ end of the detection probe or at a position 1 to 10 nt away from the 3′ end, and the reporter group and the quencher group are separated by a suitable distance so that the quencher group is able to absorb or quench the signal of the reporter group before the detection probe is hybridized to its complementary sequence. In certain preferred embodiments, the reporter group and the quencher group may be separated by a distance as defined above (e.g., a distance of 10 to 80 nt or longer). In certain preferred embodiments, one of the reporter group and the quencher group is located at the 5′ end of the detection probe and the other is located at the 3′ end.

In certain embodiments, the reporter group in the detection probe is a fluorophore (e.g., ALEX-350, FAM, VIC, TET, CAL Fluor Gold 540, JOE, HEX, CAL Fluor Orange 560, TAMRA, CAL Fluor Red 590, ROX, CAL Fluor Red 610, TEXAS RED, CAL Fluor Red 635, Quasar 670, CY3, CY5, CY5.5, Quasar 705); and, the quencher group is a molecule or group (e.g., DABCYL, BHQ (e.g., BHQ-1 or BHQ-2), ECLIPSE, and/or TAMRA) capable of absorbing/quenching the fluorescence.

In certain embodiments, the detection probe has no resistance to nuclease activity, or has resistance to nuclease activity (e.g., 5′ nuclease activity, such as 5′ to 3′ exonuclease activity); for example, the backbone of the detection probe does not contain a nuclease-resistant modification, or contains a nuclease-resistant modification, such as thiophosphate ester bond, alkylphosphotriester bond, arylphosphotriester bond, alkylphosphonate ester bond, arylphosphonate ester bond, hydrogenated phosphate ester bond, alkylaminophosphate ester bond, arylaminophosphate ester bond, 2′-O-aminopropyl modification, 2′-O-alkyl modification, 2′-O-allyl modification, 2′-O-butyl modification, and 1-(4′-thio-PD-ribofuranosyl) modification.

In certain embodiments, the detection probe is linear, or has a hairpin structure.

In certain embodiments, the detection probes each independently have the same or different reporter groups. In some embodiments, the detection probes have the same reporter group, and the product of step (b) is subjected to melting curve analysis, and then the presence of the target nucleic acid is determined according to the melting peak in the melting curve; or, the detection probes have different reporter groups, and the product of step (b) is subjected to melting curve analysis, and then the presence of the target nucleic acid is determined according to the signal type of the reporter group and the melting peak in the melting curve.

In some embodiments, in step (c), the product of step (b) is gradually heated or cooled and a signal from reporter group on each detection probe is monitored in real time, so that the curve of signal intensity of each reporting group as a function of temperature is obtained. For example, the product of step (2) can be gradually heated from a temperature of 45° C. or lower (e.g., no more than 45° C., no more than 40° C., no more than 35° C., no more than 30° C., no more than 25° C.) to 75° C.° C. or higher (e.g., at least 75° C., at least 80° C., at least 85° C., at least 90° C., at least 95° C.), and the signal sent by the reporter group on the detection probe is monitored in real time, so as to obtain the curve of signal intensity of the reporter group as a function of temperature. The rate of temperature increase can be routinely determined by one skilled in the art. For example, the rate of temperature increase can be: 0.01 to 1° C. (e.g., 0.01 to 0.05° C., 0.05 to 0.1° C., 0.1 to 0.5° C., 0.5 to 1° C., 0.04 to 0.4° C., such as 0.01° C., 0.02° C., 0.03° C., 0.04° C., 0.05° C., 0.06° C., 0.07° C., 0.08° C., 0.09° C., 0.1° C., 0.2° C., 0.3° C., 0.4° C., 0.5° C., 0.6° C., 0.7° C., 0.8° C., 0.9° C. or 1.0° C.) per step, and each step is maintained for 0.5 to 15 s (e.g., 0.5 to 1 s, 1 to 2 s, 2 to 3 s, 3 to 4 s, 4 to 5 s, 5 to 10 s, 10 to 15 s); or the temperature is elevated by 0.01 to 1° C. (e.g., 0.01 to 0.05° C., 0.05 to 0.1° C., 0.1 to 0.5° C., 0.5 to 1° C., 0.04 to 0.4° C., for example, 0.01° C., 0.02° C., 0.03° C., 0.04° C., 0.05° C., 0.06° C., 0.07° C., 0.08° C., 0.09° C., 0.1° C., 0.2° C., 0.3° C., 0.4° C., 0.5° C., 0.6° C., 0.7° C., 0.8° C., 0.9° C. or 1.0° C.) per second.

The curve is then derived to obtain a melting curve for the product of step (b).

In certain embodiments, the genotype of each SNP site is determined according to the melting peak (melting point) in the melting curve.

In certain embodiments, the detection probes comprise detection probes having nucleotide sequences selected from the following of any combination thereof (e.g., any combination of 5, 10, 15, 20, 23): SEQ ID NOs: 3, 6, 9, 12, 15, 18, 21, 24, 27, 30, 33, 36, 39, 42, 45, 48, 51, 54, 57, 60, 63, 66 and 69.

In certain embodiments, in step (a) of the method, there is provided with 1 to 5, 5 to 10, 10 to 15, 15 to 20 or more target-specific primer pairs.

In certain embodiments, in step (b) of the method, the first universal primer and the second universal primer have a working concentration higher than the working concentrations of the forward primer and the reverse primer; for example, the first universal primer and the second universal primer have a working concentration 1 to 5 times, 5 to 10 times, 10 to 15 times, 15 to 20 times, 20 to 50 times or more times higher than the working concentrations of the forward primer and the reverse primer.

In some embodiments, in step (b) of the method, the first universal primer and the second universal primer have the same working concentration; or, the first universal primer has a working concentration lower than that of the second universal primer.

In certain embodiments, in step (b) of the method, the forward primer and the reverse primer have the same or different working concentrations.

In certain embodiments, the sample or target nucleic acid comprises a mRNA, and the sample is subjected to a reverse transcription reaction prior to step (b) of the method.

In certain embodiments, in step (b) of the method, a nucleic acid polymerase (especially a template-dependent nucleic acid polymerase) is used to perform the PCR reaction. In certain embodiments, the nucleic acid polymerase is a DNA polymerase, for example, a thermostable DNA polymerase. In certain embodiments, the thermostable DNA polymerase is obtained from, Thermus aquaticus (Taq), Thermus thermophiles (Tth), Thermus filiformis, Thermis flavus, Thermococcus literalis, Thermus antranildanii, Thermus caldophllus, Thermus chliarophilus, Thermus flavus, Thermus igniterrae, Thermus lacteus, Thermus oshimai, Thermus ruber, Thermus rubens, Thermus scotoductus, Thermus silvanus, Thermus thermophllus, Thermotoga maritima, Thermotoga neapolitana, Thermosipho africanus, Thermococcus litoralis, Thermococcus barossi, Thermococcus gorgonarius, Thermotoga maritima, Thermotoga neapolitana, Thermosipho africanus, Pyrococcus woesei, Pyrococcus horikoshii, Pyrococcus abyssi, Pyrodictium occultum, Aquifex pyrophilus and Aquifex aeolieus. In certain embodiments, the DNA polymerase is Taq polymerase.

In some embodiments, the first universal primer consists of the first universal sequence, or comprises the first universal sequence and an additional sequence, and the additional sequence is located at the 5′ end of the first universal sequence. In certain embodiments, the additional sequence comprises 1 to 5, 5 to 10, 10 to 15, 15 to 20 or more nucleotides.

In certain embodiments, the first universal sequence is located at or forms the 3′ portion of the first universal primer.

In the embodiment of the present application, the first universal primer can be of any length as long as it can perform PCR reaction. In certain embodiments, the first universal primer has a length of 5 to 15 nt, 15 to 20 nt, 20 to 30 nt, 30 to 40 nt, or 40 to 50 nt.

In certain embodiments, the first universal primer or any constituent thereof comprises or consists of naturally occurring nucleotides (e.g., deoxyribonucleotides or ribonucleotides), modified nucleotides, non-natural nucleotides, or any combination thereof. In certain preferred embodiments, the first universal primer (or any constituent thereof) comprises or consists of natural nucleotides (e.g., deoxyribonucleotides or ribonucleotides). In certain preferred embodiments, the first universal primer (or any constituent thereof) comprises a modified nucleotide, such as a modified deoxyribonucleotide or ribonucleotide, such as 5-methylcytosine or 5-hydroxymethylcytosine. In certain preferred embodiments, the first universal primer (or any constituent thereof) comprises a non-natural nucleotide such as deoxyinosine, inosine, 1-(2′-deoxy-β-D-ribofuranosyl)-3-nitropyrrole, 5-nitroindole or locked nucleic acid (LNA).

In some embodiments, the second universal primer consists of the second universal sequence, or alternatively, comprises the second universal sequence and an additional sequence, and the additional sequence is located at the 5′ end of the second universal sequence. In certain embodiments, the additional sequence comprises 1 to 5, 5 to 10, 10 to 15, 15 to 20 or more nucleotides.

In certain embodiments, the second universal sequence is located at or forms the 3′ portion of the second universal primer.

In some embodiments, the second universal sequence comprises the first universal sequence and additionally comprises 1 to 5, 5 to 10, 10 to 15, 15 to 20 or more nucleotides at the 3′ end of the first universal sequence.

In the embodiment of the present application, the second universal primer can be of any length as long as it can carry out PCR reaction. In certain embodiments, the second universal primer has a length of 8 to 15 nt, 15 to 20 nt, 20 to 30 nt, 30 to 40 nt, or 40 to 50 nt.

In certain embodiments, the second universal primer or any constituent thereof comprises or consists of naturally occurring nucleotides (e.g., deoxyribonucleotides or ribonucleotides), modified nucleotides, non-natural nucleotides, or any combination thereof. In certain preferred embodiments, the second universal primer (or any constituent thereof) comprises or consists of natural nucleotides (e.g., deoxyribonucleotides or ribonucleotides). In certain preferred embodiments, the second universal primer (or any constituent thereof) comprises a modified nucleotide, such as a modified deoxyribonucleotide or ribonucleotide, such as 5-methylcytosine or 5-hydroxymethylcytosine. In certain preferred embodiments, the second universal primer (or any constituent thereof) comprises a non-natural nucleotide such as deoxyinosine, inosine, 1-(2′-deoxy-β-D-ribofuranosyl)-3-nitropyrrole, 5-nitroindole or locked nucleic acid (LNA).

In some embodiments, in the forward primer, the forward nucleotide sequence is directly ligated to the 3′ end of the first universal sequence, or ligated through a nucleotide linker to the 3′ end of the first universal sequence. In certain embodiments, the nucleotide linker comprises 1 to 5, 5 to 10, 10 to 15, 15 to 20 or more nucleotides.

In certain embodiments, the forward primer further comprises an additional sequence located at the 5′ of the first universal sequence. In certain embodiments, the additional sequence comprises 1 to 5, 5 to 10, 10 to 15, 15 to 20 or more nucleotides.

In certain embodiments, the forward primer comprises or consists of the first universal sequence and the forward nucleotide sequence from 5′ to 3′; or, comprises or consists of the first universal sequence, the nucleotide linker and the forward nucleotide sequence from 5′ to 3′; or, comprises or consists of the additional sequence, the first universal sequence and the forward nucleotide sequence from 5′ to 3′; or, comprises or consists of the additional sequence, the first universal sequence, the nucleotide linker and the forward nucleotide sequence from 5′ to 3′.

In certain embodiments, the forward nucleotide sequence is located at or constitutes the 3′ portion of the forward primer.

In some embodiments, the forward nucleotide sequence has a length of 10 to 20 nt, 20 to 30 nt, 30 to 40 nt, 40 to 50 nt, 50 to 60 nt, 60 to 70 nt, 70 to 80 nt, 80 to 90 nt, 90 to 100 nt.

In certain embodiments, the forward primer has a length of 15 to 20 nt, 20 to 30 nt, 30 to 40 nt, 40 to 50 nt, 50 to 60 nt, 60 to 70 nt, 70 to 80 nt, 80 to 90 nt, 90 to 100 nt, 100 to 110 nt, 110 to 120 nt, 120 to 130 nt, 130 to 140 nt, 140 to 150 nt.

In certain embodiments, the forward primer, or any constituent thereof, comprises or consists of naturally occurring nucleotides (e.g., deoxyribonucleotides or ribonucleotides), modified nucleotides, non-natural Nucleotides, or any combination thereof. In certain preferred embodiments, the forward primer (or any constituent thereof) comprises or consists of natural nucleotides (e.g., deoxyribonucleotides or ribonucleotides). In certain preferred embodiments, the forward primer (or any constituent thereof) comprises a modified nucleotide, such as modified deoxyribonucleotide or ribonucleotide, such as 5-methylcytosine or 5-hydroxymethylcytosine. In certain preferred embodiments, the forward primer (or any constituent thereof) comprises a non-natural nucleotide, such as deoxyinosine, inosine, 1-(2′-deoxy-β-D-ribofuranosyl)-3-nitropyrrole, 5-nitroindole or locked nucleic acid (LNA).

In some embodiments, in the reverse primer, the reverse nucleotide sequence is directly ligated to the 3′ end of the second universal sequence, or the reverse nucleotide sequence is ligated through a nucleotide linker to the 3′ end of the second universal sequence. In certain embodiments, the nucleotide linker comprises 1 to 5, 5 to 10, 10 to 15, 15 to 20 or more nucleotides.

In certain embodiments, the reverse primer further comprises an additional sequence located at the 5′ of the second universal sequence. In certain embodiments, the additional sequence comprises 1 to 5, 5 to 10, 10 to 15, 15 to 20 or more nucleotides.

In some embodiments, the reverse primer comprises or consists of the second universal sequence and the reverse nucleotide sequence from 5′ to 3′; or, comprises or consists of the second universal sequence, the nucleotide linker and the reverse nucleotide sequence from 5′ to 3′; or, comprises or consists of the additional sequence, the second universal sequence and the reverse nucleotide sequence from 5′ to 3′; or, comprises or consists of the additional sequence, the second universal sequence, the nucleotide linker and the reverse nucleotide sequence from 5′ to 3′.

In certain embodiments, the reverse nucleotide sequence is located at or constitutes the 3′ portion of the reverse primer.

In some embodiments, the reverse nucleotide sequence has a length of 10 to 20 nt, 20 to 30 nt, 30 to 40 nt, 40 to 50 nt, 50 to 60 nt, 60 to 70 nt, 70 to 80 nt, 80 to 90 nt, 90 to 100 nt.

In certain embodiments, the primer has a length of is 15 to 20 nt, 20 to 30 nt, 30 to 40 nt, 40 to 50 nt, 50 to 60 nt, 60 to 70 nt, 70 to 80 nt, 80 to 90 nt, 90 to 100 nt, 100 to 110 nt, 110 to 120 nt, 120 to 130 nt, 130 to 140 nt, 140 to 150 nt.

In certain embodiments, the reverse primer, or any constituent thereof, comprises or consists of naturally occurring nucleotides (e.g., deoxyribonucleotides or ribonucleotides), modified nucleotides, non-natural Nucleotides, or any combination thereof. In certain preferred embodiments, the reverse primer (or any constituent thereof) comprises or consists of natural nucleotides (e.g., deoxyribonucleotides or ribonucleotides). In certain preferred embodiments, the reverse primer (or any constituent thereof) comprises a modified nucleotide, such as modified deoxyribonucleotide or ribonucleotide, such as 5-methylcytosine or 5-hydroxymethylcytosine. In certain preferred embodiments, the reverse primer (or any constituent thereof) comprises a non-natural nucleotide such as deoxyinosine, inosine, 1-(2′-deoxy-β-D-ribofuranosyl)-3-nitropyrrole, 5-nitroindole or locked nucleic acid (LNA).

In some embodiments, the second universal sequence cannot be completely complementary to the complementary sequence of the forward primer; for example, at least one nucleotide, such as 1 to 5, 5 to 10, 10 to 15, 15 to 20 or more nucleotides located at the 3′ end of the second universal sequence cannot be complementary to the complementary sequence of the forward primer.

In some embodiments, the sequence of the first universal primer is set forth in SEQ ID NO: 71.

In some embodiments, the sequence of the second universal primer is set forth in SEQ ID NO: 70.

In certain embodiments, the target-specific primer pair comprises a primer pair having nucleotide sequences selected from the following or any combination thereof (e.g., any 5 pairs, 10 pairs, 15 pairs, 20 pairs, 23 combination): SEQ ID NOs: 1 and 2; 4 and 5; 7 and 8; 10 and 11; 13 and 14; 16 and 17; 19 and 20; 22 and 23; 34 and 35; 37 and 38; 40 and 41; 43 and 44; 46 and 47; 49 and 50; 52 and 53; 55 and 56; 58 and 59; 61 and 62; 64 and 65; 67 and 68.

In certain embodiments, the target SNP sites are each independently selected from:
(1) an SNP site at which the donor has a first homozygous genotype and the recipient has a second homozygous genotype;
(2) an SNP site at which the donor has a homozygous genotype and the recipient has a heterozygous genotype.

In certain preferred embodiments, the ratio of the donor in the sample from the recipient is calculated by scheme (1).

In certain embodiments, the ratio of the donor in the sample from the recipient is calculated by one or more of the following methods:
(1) when the target SNP site is an SNP site at which the donor has a first homozygous genotype (e.g., AA) and the recipient has a second homozygous genotype (e.g., BB), the ratio of the donor in the sample from the recipient is:

$$donor chimerism rate = 1 - \frac{{N}_{B}}{{N}_{A} + {N}_{B}}$$

wherein, N_{B} is the copy number of allele B (which can be determined by digital PCR), and N_{A} is the copy number of allele A (which can be determined by digital PCR);
(2) when the target SNP site is an SNP site at which the donor has a homozygous genotype (e.g., AA), and the recipient has a heterozygous genotype (e.g., AB), the ratio of the donor in the sample from the recipient is:

$$donor chimerism rate = 1 - \frac{2 {N}_{B}}{{N}_{A} + {N}_{B}}$$

wherein, N_{B} is the copy number of allele B (which can be determined by digital PCR), and N_{A} is the copy number of allele A (which can be determined by digital PCR).

In certain embodiments, the transplant is an organ transplant.

In certain embodiments, the organ transplant is selected from kidney, heart, lung, liver, pancreas, or any combination thereof.

In certain embodiments, the recipient sample is selected from the blood (e.g., peripheral blood), urine, and any combination thereof from the recipient after transplantation.

In certain embodiments, the target SNP sites are each independently selected from:
(1) an SNP site at which the donor has a first homozygous genotype and the recipient has a second homozygous genotype;
(2) an SNP site at which the donor has a heterozygous genotype and the recipient has a homozygous genotype.

In certain preferred embodiments, the ratio of the donor in the sample of the recipient is calculated by scheme (1).

In certain embodiments, the ratio of the donor in the sample from the recipient is calculated by one or more of the following methods:
(1) when the target SNP site is an SNP site at which the donor has a first homozygous genotype (e.g., BB) and the recipient has a second homozygous genotype (e.g., AA), the ratio of the donor in the sample from the recipient is:

$$dd - cf DNA \% = \frac{{N}_{B}}{{N}_{A} + {N}_{B}}$$

wherein, N_{B} is the copy number of allele B (which can be determined by digital PCR), and N_{A} is the copy number of allele A (which can be determined by digital PCR);
(2) when the target SNP site is an SNP site at which the donor has a heterozygous genotype (e.g., AB) and the recipient has a homozygous genotype (e.g., AA), the ratio of the donor in the sample from the recipient is:

$$dd - cf DNA \% = \frac{2 {N}_{B}}{{N}_{A} + {N}_{B}}$$

wherein, N_{B} is the copy number of allele B (which can be determined by digital PCR), and N_{A} is the copy number of allele A (which can be determined by digital PCR).

In the method of the present application, taking the first probe in the probe set as an example, it can be hybridized or annealed (preferably completely complementary) to the nucleic acid molecule having the first allele. Therefore, when performing the digital PCR reaction, during annealing or extension process, the first probe will form a duplex with the nucleic acid molecule and be degraded by a nucleic acid polymerase (e.g., a DNA polymerase) during amplification, to release a reporter group (e.g., a fluorophore). Thus, after the digital PCR amplification reaction is completed, the endpoint fluorescence of each droplet is detected by a droplet detector, and according to the signal (e.g., a first fluorescence signal) intensity of the free first reporter group (e.g., a first fluorophore), the numbers of positive droplets and negative droplets can be determined, thereby determining the amount of the nucleic acid molecule having the first allele in the sample. Similarly, after the digital PCR amplification reaction is completed, the endpoint fluorescence of each droplet is detected by a droplet detector, and according to the signal (e.g., a second fluorescence signal) intensity of the free second reporter group (e.g., a second fluorophore), the numbers of positive droplets and negative droplets can be determined, thereby determining the amount of the nucleic acid molecule having the second allele in the sample. Since the genotypes of the donor/recipient are different, the contents corresponding to the first/second alleles are different. Therefore, by comparing and analyzing the amounts of nucleic acid molecules containing the first/second alleles, the presence or absence of the donor in the sample from the recipient can be determined, and optionally, the ratio of the donor is determined.

In the method of the present application, in some embodiments, the first probe is not annealed or hybridized to the nucleic acid molecule having the second allele during the digital PCR reaction; and/or, the second probe is not annealed or hybridized to the nucleic acid molecule having the first allele during the digital PCR reaction. It is easy to understand that the hybridization specificity of the first/second probe is particularly advantageous, which can help to accurately determine the content of the first allele/second allele, thereby helping to calculate the ratios of the donor sample and the recipient sample, respectively. In some embodiments, the hybridization specificity of the first/second probe can be obtained by controlling the annealing temperature and/or the extension temperature of the digital PCR reaction. For example, the annealing temperature and/or extension temperature can be set to be lower than the melting point of the duplex formed by the first probe and the nucleic acid molecule having the first allele, but higher than the melting point of the duplex formed by the first probe and the nucleic acid molecule having the second allele, such that the first probe can be hybridized to the nucleic acid molecule having the first allele but cannot be hybridized to the nucleic acid molecule having the second allele during the digital PCR reaction. Similarly, the annealing temperature and/or extension temperature can be set to be lower than the melting point of the duplex formed by the second probe and the nucleic acid molecule having the second allele, but higher than the melting point of the duplex formed by the second probe and the nucleic acid molecule having the first allele, such that the second probe can be hybridized to the nucleic acid molecule having the second allele but cannot be hybridized to the nucleic acid molecule having the first allele during the digital PCR reaction.

In the method of the present application, the copy numbers of alleles can be detected by the digital PCR platform and directly output by the software according to the Poisson distribution principle. The relevant principles and calculation methods can be found in, for example, Milbury C A, Zhong Q, Lin J, et al. Determining lower limits of detection of digital PCR assays for cancer to related gene mutations. Biomol Detect Quantif 2014; 1(1):8 to 22. Published 2014 August 20. doi:10.1016/j.bdq.2014.08.001.

In the seventh aspect, the present application provides a kit, the kit comprising an identification primer set capable of asymmetrically amplifying a target nucleic acid containing a candidate SNP site.

In some embodiments, the identification primer set comprises: a first universal primer and a second universal primer, and, for each candidate SNP site, at least one target-specific primer pair is provided, wherein,
the first universal primer comprises a first universal sequence;
the second universal primer comprises a second universal sequence, the second universal sequence comprises the first universal sequence and additionally comprises at least one nucleotide at the 3′ end of the first universal sequence;
the target-specific primer pair is capable of performing amplification using the target nucleic acid as a template to generate a nucleic acid product containing the candidate SNP site, and the target-specific primer pair comprises a forward primer and a reverse primer, wherein, the forward primer comprises the first universal sequence and a forward nucleotide sequence specific to the target nucleic acid, and the forward nucleotide sequence is located at the 3′ end of the first universal sequence; the reverse primer comprises the second universal sequence and a reverse nucleotide sequence specific to the target nucleic acid, and the reverse nucleotide sequence is located at the 3′ end of the second universal sequence; and, the second universal sequence is not completely complementary to the complementary sequence of the forward primer.

In some embodiments, the kit further comprises one or more detection probes capable of detecting the candidate SNP site, the detection probe comprises a nucleotide sequence specific to the target nucleic acid and is capable of annealing or hybridizing to a region of the target nucleic acid containing the candidate SNP site, and is labeled with a reporter group and a quencher group, wherein the reporter group is capable of emitting a signal, and the quencher group is capable of absorbing or quenching the signal emitted by the reporter group; and the signal emitted by the detection probe when it is hybridized to its complementary sequence is different from the signal sent when it is not hybridized to its complementary sequence.

In some embodiments, the candidate SNP site has one or more features selected from the following:
(1) the candidate SNP has a Fst of less than 0.3 (e.g., less than 0.2, less than 0.1, less than 0.05, less than 0.01) between different human races;
(2) the candidate SNP site is located on different chromosomes;
(3) the candidate SNP site has an allele frequency between 0.2 and 0.8 (e.g., between 0.3 and 0.7, between 0.4 and 0.6).

In some embodiments, the candidate SNP site has one or more features selected from the following:
(1) the candidate SNP site has a Fst of less than 0.01 between different human races;
(2) the candidate SNP site is located on different chromosomes;
(3) the candidate SNP site has an allele frequency between 0.3 and 0.7.

In certain embodiments, the candidate SNP site is an SNP site with a biallelic polymorphism.

In certain embodiments, the candidate SNP site is an SNP site in the human genome; for example, the target nucleic acid comprises a human genome SNP site selected from the group consisting of: rs16363, rs1610937, rs5789826, rs1611048, rs2307533, rs112552066, rs5858210, rs2307839, rs149809066, rs66960151, rs34765837, rs68076527, rs10779650, rs4971514, rs6424243, rs12990278, rs2122080, rs98506667, rs774763, rs711725, rs2053911, rs9613776, rs7160304, and any combination of the aforementioned SNP sites (e.g., combination of any 5, 10, 15, 20, 23 of the aforementioned SNP sites).

In certain embodiments, the target nucleic acid comprises a human genome SNP site selected from the group consisting of: rs16363, rs1610937, rs5789826, rs1611048, rs2307533, rs112552066, rs5858210, rs2307839, rs149809066, rs66960151, rs34765837, rs68076527, rs10779650, rs4971514, rs6424243, rs12990278, rs2122080, rs98506667, rs774763, rs711725, rs2053911, rs9613776 and rs7160304.

In certain embodiments, the detection probe comprises a detection probe having nucleotide sequence selected from or any combination thereof (e.g., any combination of 5, 10, 15, 20, 23): SEQ ID NOs: 3, 6, 9, 12, 15, 18, 21, 24, 27, 30, 33, 36, 39, 42, 45, 48, 51, 54, 57, 60, 63, 66 and 69.

In some embodiments, the sequence of the first universal primer is set forth in SEQ ID NO: 71.

In some embodiments, the sequence of the second universal primer is set forth in SEQ ID NO: 70.

In certain embodiments, the target-specific primer pair comprises a primer pair having nucleotide sequences selected from the following or any combination thereof (e.g., any 5 pairs, 10 pairs, 15 pairs, 20 pairs, 23 combination): SEQ ID NOs: 1 and 2; 4 and 5; 7 and 8; 10 and 11; 13 and 14; 16 and 17; 19 and 20; 22 and 23; 34 and 35; 37 and 38; 40 and 41; 43 and 44; 46 and 47; 49 and 50; 52 and 53; 55 and 56; 58 and 59; 61 and 62; 64 and 65; 67 and 68.

It is easy to understand that the first universal primer, the second universal primer, the target-specific primer pair and the detection probe in the kit of the present application are used to implement the method as described above. Therefore, the detailed descriptions above for the first universal primer, the second universal primer, the target-specific primer pair and the detection probe (including the description of various preferred features and exemplary features) are also applicable here.

In certain embodiments, the kit further comprises one or more components selected from the group consisting of: an amplification primer set, a probe set, and reagents for performing the digital PCR.

In some embodiments, the amplification primer set comprises at least one amplification primer (e.g., a pair of amplification primers or more amplification primers), which can specifically amplify a nucleic acid molecule containing the SNP site under a condition allowing nucleic acid hybridization or annealing.

In certain embodiments, the probe set comprises a first probe and a second probe; wherein,
(i) the first probe and the second probe are each independently labeled with a reporter group and a quencher group, wherein the reporter group can emit a signal, and the quencher group is capable of absorbing or quenching the signal emitted by the reporter group; and, the first probe and the second probe are respectively labeled with different reporter groups (e.g., fluorophores); and
(ii) the first probe is capable of hybridizing or annealing (preferably completely complementary) to a nucleic acid molecule containing the first allele of the target SNP site, and the second probe is capable of hybridizing or annealing (preferably completely complementary) to a nucleic acid molecule containing the second allele of the target SNP site; and, the first probe and the second probe are specific for different alleles.

In certain embodiments, the probe set comprises probes having nucleotide sequences selected from the following or any combination thereof (e.g., any combination of 5, 10, 20, 40, 60): SEQ ID NOs: 73, 74, 78, 79, 82, 83, 86, 87, 90, 91, 94, 95, 98, 99, 102, 103, 106, 107, 110, 111, 114, 115, 118, 119, 122, 123, 126, 127, 130, 131, 134, 135, 138, 139, 142, 143, 146, 147, 150, 151, 154, 155, 158, 159, 162, 163.

In some embodiments, the amplification primer set comprises a primer pair having nucleotide sequences selected from the following or any combination thereof (e.g., any combination of 5 pairs, 10 pairs, 15 pairs, 20 pairs, 23 pairs): SEQ ID NOs: 72 and 73; 77 and 76; 80 and 81; 84 and 85; 88 and 89; 92 and 93; 96 and 97; 100 and 101; 104 and 105; 108 and 109; 116 and 117; 120 and 121; 124 and 125; 128 and 129; 132 and 133; 136 and 137; 140 and 141; 144 and 145; 148 and 149; 152 and 153; 156 and 157; 160 and 161.

In certain embodiments, the reagents for performing the digital PCR are one or more components selected from the group consisting of: reagents for preparing droplet samples, reagents for nucleic acid amplification, nucleic acid polymerase, reagents for detecting droplet samples, or any combination thereof.

In certain embodiments, the kit further comprises one or more components selected from the group consisting of: nucleic acid polymerase, reagents for nucleic acid amplification, reagents for performing melting curve analysis, or any combination thereof.

It is easy to understand that the amplification primer set and the probe set (first probe and second probe) in the kit of the present application are used to implement the method as described above. Therefore, the detailed descriptions (including the descriptions of various preferred features and exemplary features) above for the amplification primer set and the probe set (first probe and second probe) are also applicable here.

In some embodiments, the nucleic acid polymerase is a template-dependent nucleic acid polymerase, such as a DNA polymerase, particularly a thermostable DNA polymerase; in some embodiments, the nucleic acid polymerase is as defined above.

In certain embodiments, the reagents for performing nucleic acid amplification comprise, working buffers for enzymes (e.g., nucleic acid polymerase), dNTPs (labeled or unlabeled), water, solutions containing ions (e.g., Mg²⁺), single-stranded DNA binding protein, or any combination thereof.

In some embodiments, the kit is used to determine whether a sample from the recipient contains the donor, or to calculate the ratio of the donor in a sample from the recipient.

In certain embodiments, the digital PCR is selected from droplet digital PCR and chip digital PCR.

In certain embodiments, the present application provides a use of the identification primer set as described above in the manufacture of a kit, in which the kit is used for asymmetrically amplifying a target nucleic acid molecule, or for detecting a genotype of a candidate SNP site in a target nucleic acid molecule; or for identifying an SNP site at which the donor and the recipient have different genotypes; or for identifying an SNP site at which the recipient has a homozygous allele.

In certain embodiments, the kit further comprises a detection probe as defined above.

In certain embodiments, the kit is used to implement the method as previously described.

In certain embodiments, the present application provides a use of the amplification primer set and the probe set as described above in the manufacture of a kit, in which the kit is used for detecting the presence or proportion of nucleic acids from the donor in a sample from the recipient after transplantation.

In certain embodiments, the kit further comprises reagents for determining a genotype of one or more SNP sites in the genome of the recipient or donor.

In certain embodiments, the kit further comprises the identification primer set and the detection probe as defined above.

In certain embodiments, the kit is used to implement the method as previously described.

### Beneficial Effects of the Present Invention

Compared with the prior art, the advantages of the present application are: (1) automatic detection, less manual operation steps, and short detection cycle. The unique SNP typing system of the present application can realize the typing of multiple SNPs at the same time, with a high degree of automation. The whole process from nucleic acid extraction to obtaining results can be completed within one day, and can timely measure the donor chimerism rate in a patient with transplanted bone marrow, evaluate the chimerism status of hematopoietic stem cells, and can complete the measurement of dd-cfDNA ratio in a patient with transplanted organ, thereby reflecting the healthy status of transplant; (2) high accuracy and sensitivity. It can absolutely quantify the copy number of heterologous DNA, accurately calculate the donor chimerism rate or dd-cfDNA ratio, and the detection sensitivity of heterologous DNA is as low as 0.1%. (3) Non-invasive and universal detection process: it does not rely on quantitative analysis of donor sample, and has low cost and intuitive digital quantitative results, so that the method can be widely used.

The embodiments of the present invention will be described in detail below with reference to the drawings and examples, but those skilled in the art will understand that the following drawings and examples are only for illustrating the present invention, rather than limiting the scope of the present invention. Various objects and advantages of the present invention will become apparent to those skilled in the art from the accompanying drawings and the following detailed description of the preferred embodiments.

### BRIEF DESCRIPTION OF THE DRAWINGS

FIG. 1 shows a schematic diagram for depicting an exemplary embodiment of the method of the present invention for detecting the presence or proportion of a donor in a recipient sample by SNP typing to illustrate the basic principle of the method of the present invention.

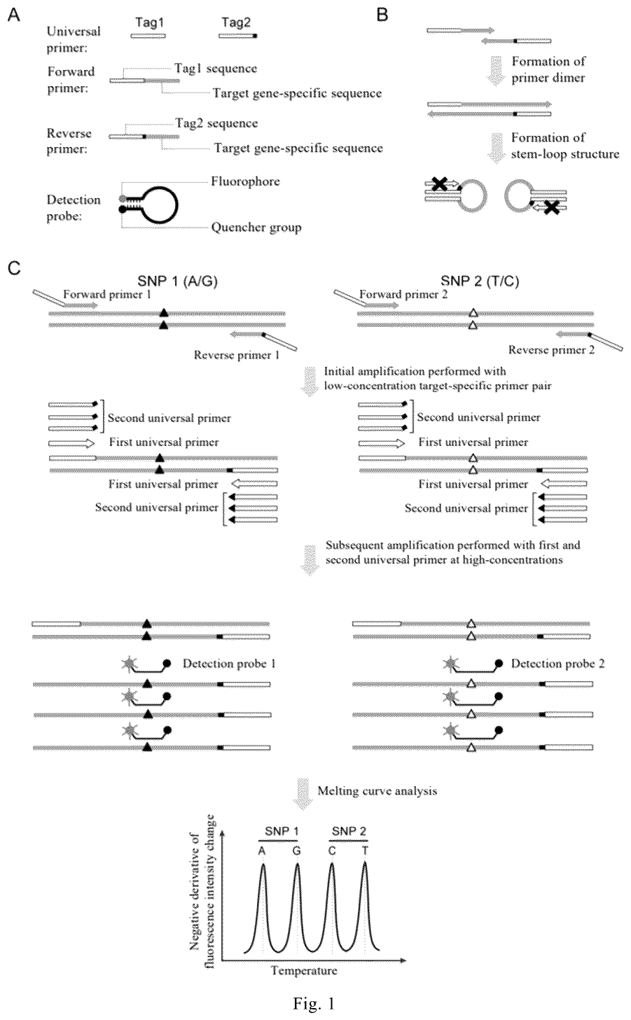

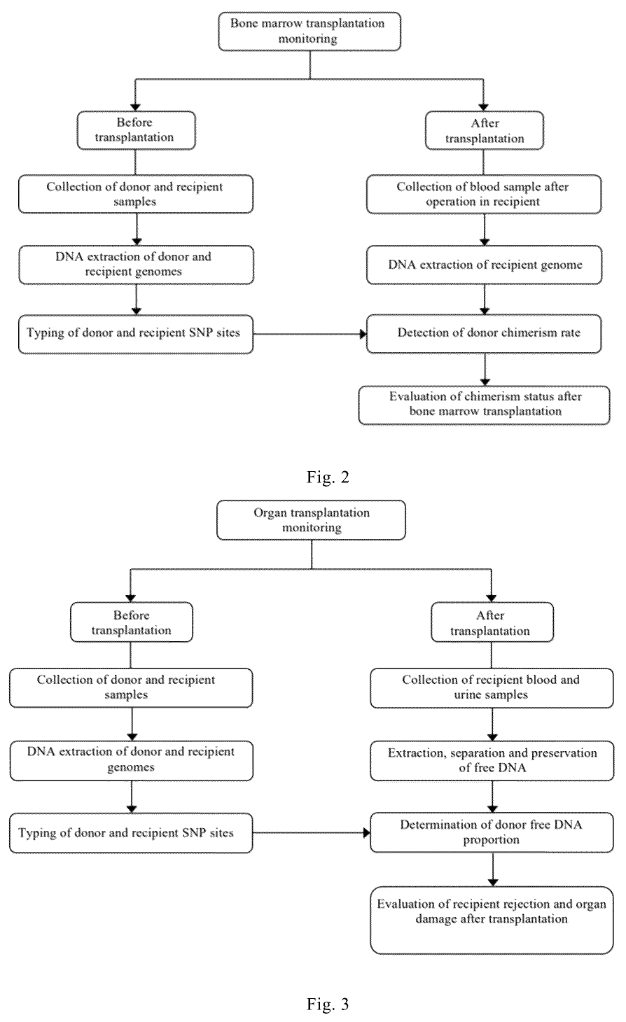

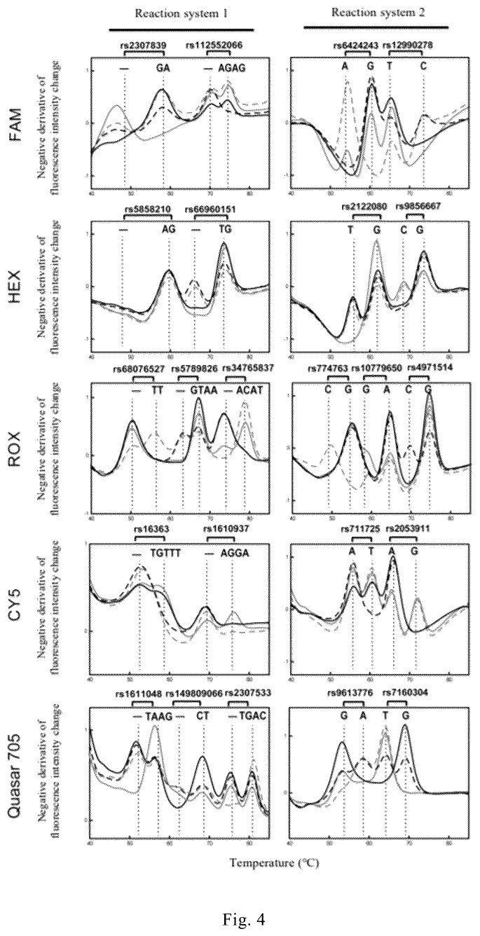

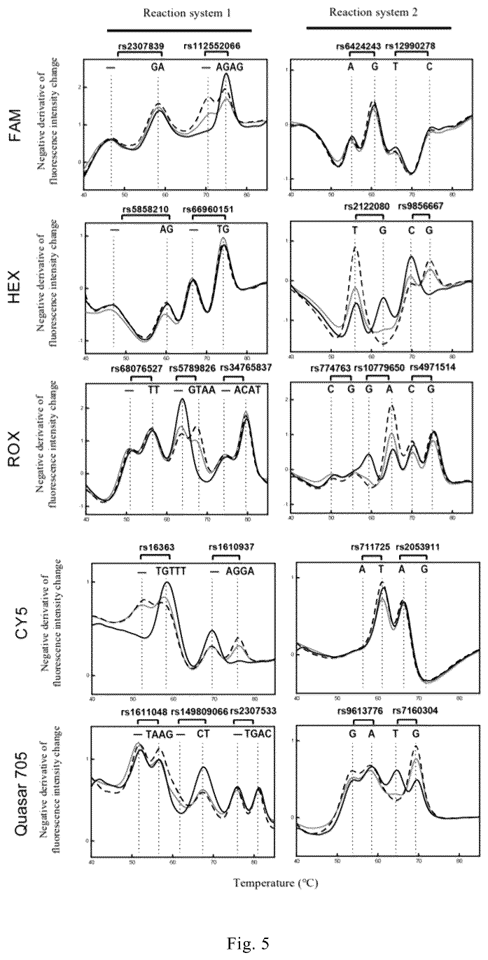

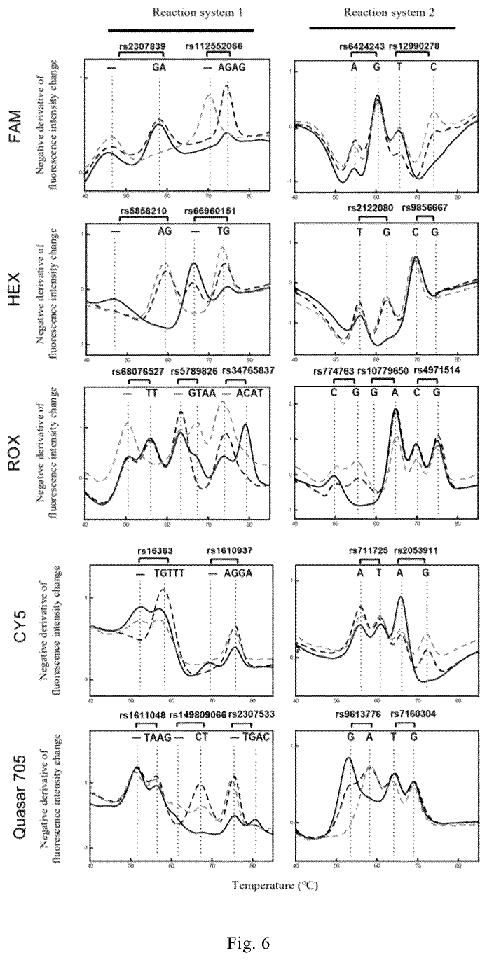

FIG. 1A shows a schematic diagram for depicting a primer set and a self-quenching fluorescent detection probe involved in this embodiment, wherein the primer set comprises: a first universal primer and a second universal primer, and a target-specific primer pair containing a forward primer and a reverse primer; wherein,
the first universal primer comprises a first universal sequence (Tag1);
the second universal primer comprises a second universal sequence (Tag2), which comprises the first universal sequence and additionally comprises at least one nucleotide (e.g., 1 to 5, 5 to 10, 10 to 15, 15 to 20 or more nucleotides) at the 3′ end of the first universal sequence;
the forward primer comprises the first universal sequence and a forward nucleotide sequence specific to the target nucleic acid containing the SNP site, and the forward nucleotide sequence is located at the 3′ end of the first universal sequence;
the reverse primer comprises the second universal sequence and a reverse nucleotide sequence specific to the target nucleic acid containing the SNP site, and the reverse nucleotide sequence is located at the 3′ end of the second universal sequence; and,
the forward primer and the reverse primer are capable of specifically amplifying the corresponding target nucleic acid containing the SNP site; and,
the second universal sequence cannot be completely complementary to the complementary sequence of the forward primer.

FIG. 1B shows a schematic diagram for depicting the principle that the non-specific amplification of primer dimers is suppressed when the primer set of FIG. 1A is used for amplification, wherein, since the primer dimers formed by the non-specific amplification of the forward primer and the reverse primer will produce a single-stranded nucleic acid with reverse sequences complementary to each other contained at its 5′ end and 3′ end after denaturation, the single-stranded nucleic acid itself will form a panhandle structure during the annealing stage, which prevents the first universal primer and the second universal primer from performing the annealing and extension of the single-stranded nucleic acid, thereby inhibiting further amplification of the primer dimers.

FIG. 1C shows a schematic diagram for depicting the principle of simultaneous detection of multiple target nucleic acids containing SNP sites using the primer set and detection probe of FIG. 1A. In this embodiment, for each target nucleic acid containing SNP site, a pair of forward primer and reverse primer, and a self-quenching fluorescent detection probe are designed, and the specific detection process is as follows:

First, PCR amplification is initiated by the target-specific primer pair at a low-concentration to generate an initial amplification product comprising two nucleic acid strands (nucleic acid strand A and nucleic acid strand B) that are complementary to the forward primer/first universal primer and reverse primer/second universal primer, respectively; subsequently, the initial amplification product is subjected to subsequent PCR amplification with the first universal primer and the second universal primer at a high-concentration.

Since the reverse primer/second universal primer contains the first universal sequence, the first universal primer not only can anneal to the nucleic acid strand A (the nucleic acid strand complementary to the forward primer/first universal primer) and synthesize its complementary strand, but also can anneal to the nucleic acid strand B (the nucleic acid strand complementary to the reverse primer/second universal primer) and synthesize its complementary strand. That is, the first universal primer can simultaneously amplify the nucleic acid strand A and the nucleic acid strand B.

The second universal primer contains an additional nucleotide at the 3′ end of the first universal primer, therefore, it does not match the nucleic acid strand A (the nucleic acid strand complementary to the forward primer/first universal primer) at the at the 3′ end (i.e., not completely complementary at the 3′ end). Thus, during the amplification process, the second universal primer will preferentially anneal to the nucleic acid strand B (the nucleic acid strand complementary to the reverse primer/second universal primer) and synthesize its complementary strand, but substantially cannot perform extension to synthesize the complementary strand of the nucleic acid strand A (the nucleic acid strand complementary to the forward primer/first universal primer).

Therefore, as the PCR amplification proceeds, the synthesis efficiency of the complementary strand (nucleic acid strand B) of the nucleic acid strand A will be significantly lower than that of the complementary strand (nucleic acid strand A) of the nucleic acid strand B, so that the complementary strand (nucleic acid strand A) of the nucleic acid strand B is synthesized and amplified in large quantities, while the synthesis and amplification of the complementary strand (nucleic acid strand B) of the nucleic acid strand A is inhibited, thereby producing a large amount of the target single-stranded product (the nucleic acid strand A, which contains a sequence complementary to the forward primer/first universal primer and a sequence of the reverse primer/second universal primer), and achieving the asymmetric amplification. In addition, in order to further enhance the asymmetry of amplification, the ratio of the first universal primer to the second universal primer can also be adjusted, so that the concentration of the first universal primer is lower than that of the second universal primer, so as to better enrich the target single-stranded product. By using multiple pairs of forward primers and reverse primers in the same reaction system, multiple target nucleic acids containing SNP sites can be asymmetrically amplified at the same time, and a large number of single-stranded target nucleic acids containing SNP sites can be generated.

After the PCR amplification is completed, multiple self-quenching fluorescent detection probes added in advance are combined with corresponding target nucleic acid single strands containing SNP sites, respectively, to form double-stranded hybrids of the detection probes and the target nucleic acid single strands. Since the double-stranded hybrids have different stability, different melting peaks can be obtained after melting curve analysis, and then the genotype of SNP in each target nucleic acid single strand can be determined according to the melting point (Tₘ) and the type of fluorophore labeled by the probe.

FIG. 2 shows a flow chart of the method of the present invention for determining a donor chimerism rate of bone marrow transplantation.

FIG. 3 shows a flow chart of the method of the present invention for determining the ratio of dd-cfDNA of organ transplantation.

FIG. 4 shows the results of melting curve analysis after the donor and recipient genomic DNAs (10 ng/μL) of the bone marrow transplantation Case 1 sample group and Case 2 sample group were amplified by using the system of the present invention in Example 2. Wherein, the black solid line represents the detection results of the donor genomic DNA in the bone marrow transplantation Case 1 sample group; the black dotted line represents the detection results of the recipient genomic DNA in the Case 1 sample group; the gray solid line represents the detection results of the donor genomic DNA in the bone marrow transplantation Case 2 sample group; and the gray dotted line represents the detection results of the recipient genomic DNA in the Case 2 sample group.

FIG. 5 shows the results of melting curve analysis after the donor and recipient genomic DNAs (10 ng/μL) of the organ transplantation Case 3 sample group was amplified by using the system of the present invention in Example 3, wherein the black solid line represents the detection results of the donor genomic DNA in the organ transplantation Case 3 sample group; the black dotted line represents the detection result of the recipient genomic DNA in the Case 3 sample group; and, the gray solid line represents the results of melting curve analysis after the urine cell-free DNA (1 ng/μL) on the third day after operation in the organ transplantation Case 3 sample group was amplified by using the system of the present invention in Example 4.

FIG. 6 shows the results of melting curve analysis after the free urine DNA (1 ng/μL) and the recipient genomic DNA (10 ng/μL) of the organ transplantation Case 4 sample group was amplified by using the system of the present invention in Example 5, wherein, the black solid line represents the detection result of postoperative urine cell-free DNA in the organ transplantation Case 4 sample group; the black dotted line represents the detection result of the recipient genomic DNA in the Case 4 sample group; and, the results of melting curve analysis after the recipient genomic DNA (10 ng/μL) of the organ transplantation Case 5 (without donor sample) was amplified by using the system of the present invention, wherein the gray dotted line represents the detection result of the recipient genomic DNA in Case 5.

### SPECIFIC MODELS FOR CARRYING OUT THE PRESENT INVENTION

The present invention will now be described with reference to the following examples, which are intended to illustrate the present invention (not limit the present invention). It should be understood that these examples are only used to illustrate the principles and technical effects of the present invention, but do not represent all possibilities of the present invention. The present invention is not limited to the materials, reaction conditions or parameters mentioned in these examples. Those skilled in the art can use other similar materials or reaction conditions to implement other technical solutions according to the principles of the present invention. Such technical solutions do not deviate from the basic principles and concepts described in the present invention, and fall within the scope of the present invention.

### Example 1. Selection of Candidate SNP Sites

The SNP sites covered by the present invention were selected from the single nucleotide polymorphism site library (dbSNP) of the National Center for Biotechnology Information (NCBI) in the United States of America, and the SNP sites of the present invention preferably met the following conditions: (1) Fst (population fixation coefficient) between different human races was <0.01, that was, the degree of differentiation of these sites in different human races was very small, and the level of gene heterozygosity was close; (2) allele frequency was between 0.3 and 0.7; (3) the distribution in the Asian population followed Hardy-Weinberg equilibrium; (4) the distance between every two SNPs was >1 Mb; (5) in order to avoid linkage between different sites, sites on different chromosomes were selected as much as possible. SNP sites were screened according to the above criteria. In this example, 23 preferred SNP sites were selected, as shown in Table 1. The information and sequences of SNP sites were queried and downloaded from the dbSNP database of the National Center for Biotechnology Information (NCBI) in the United States of America, the allele frequencies were referred to the Asian population frequencies from the Thousand Genomes Project database, and these sites were evenly distributed on each chromosome of the genome.

TABLE 1
Information of SNP sites selected in Example 1

| | Allele | Allele | |
|---|---|---|---|
| SNP name | type | frequency | Chromosome |
| rs16363 | TGTTT/− | 0.4740/0.5260 | 22 |
| rs1610937 | AGGA/− | 0.4300/0.5700 | 5 |
| rs5789826 | GTAA/− | 0.4440/0.5560 | 11 |
| rs1611048 | TAAG/− | 0.4300/0.5700 | 7 |
| rs2307533 | TGAC/− | 0.5790/0.4210 | 14 |
| rs112552066 | AGAG/− | 0.4940/0.5060 | 1 |
| rs5858210 | AG/− | 0.4380/0.5620 | 4 |
| rs2307839 | GA/− | 0.4700/0.5300 | 6 |
| rs149809066 | CT/− | 0.4680/0.5320 | 2 |
| rs66960151 | TG/− | 0.4900/0.5100 | 12 |
| rs34765837 | ACAT/− | 0.4554/0.5446 | 10 |
| rs68076527 | TT/− | 0.5258/0.4742 | 7 |
| rs10779650 | G/A | 0.3353/0.6647 | 1 |
| rs4971514 | G/C | 0.6399/0.3601 | 2 |
| rs6424243 | G/A | 0.5873/0.4127 | 1 |
| rs12990278 | T/C | 0.7113/0.2887 | 2 |
| rs2122080 | A/C | 0.5009/0.4991 | 2 |
| rs98506667 | C/G | 0.6348/0.3652 | 3 |
| rs774763 | C/G | 0.5654/0.4346 | 3 |
| rs711725 | A/T | 0.3740/0.6260 | 3 |
| rs2053911 | G/A | 0.8392/0.1608 | 16 |
| rs9613776 | A/G | 0.5645/0.4355 | 22 |
| rs7160304 | G/T | 0.5109/0.4891 | 14 |

### Example 2. Determination of Donor Chimerism Rate after Bone Marrow Transplantation

The detection process of this example was shown in FIG. 2. Taking two bone marrow transplant sample groups as examples, the following two parts of samples were collected: 1. the donor samples and recipient samples of bone marrow transplant patients before transplantation were collected and extracted, and used for SNP typing, and the principle of SNP typing was shown in FIG. 1; 2. the peripheral blood was collected at various time points during the period of monitoring the recipient after transplantation, the genomic DNA was extracted for quantification of target SNP sites, detection of donor chimerism rate after bone marrow transplantation, and evaluation of chimerism state after allogeneic hematopoietic stem cell transplantation.

The specific operation steps of the above detection process were as follows:

1. Two bone marrow transplantation sample groups were collected (each group included donor samples and recipient samples before transplantation, and recipient samples at various time points after transplantation), in which the blood samples were collected using EDTA anticoagulant tubes (Zhejiang Gongdong Medical Technology Co., Ltd., Taizhou) and stored at 4° C.; the saliva samples were collected with a saliva collector (Xiamen Zeesan Biotech Co., Ltd., Xiamen) according to the instructions and stored at room temperature.

2. Lab-Aid 824 Nucleic Acid Extraction System and supporting reagents (Xiamen Zeesan Biotech Co., Ltd., Xiamen) for extraction of blood and saliva genomic DNAs were used to extract the genomic DNAs of the above blood and saliva samples, and Nanodrop 2000 spectrophotometer (Thermo Fisher Scientific, USA) was used to determine the concentrations and purities of the genomic DNAs.

3. SNP Typing

According to the selected SNP sites, corresponding primers and probes were designed, and the multiple asymmetric PCR system of the present invention (the principle was shown in FIG. 1) was used for typing 23 SNPs in 2 PCR reaction systems at the same time, and the sequences and concentrations of primers and probes used were shown in Table 2.

TABLE 2
Sequences and concentrations of the primers and probes used in Example 2

| Reaction | | | | SEQ |
|---|---|---|---|---|
| system | Name | Sequence (5′-3′) | Concentration | ID No. |
| 1 | rs2307839-F | Tag1-TAGGTAATCTGAGGTGGCATC | 0.04 | 1 |
| | rs2307839-R | Tag2-TGTAATTTCCTACCTAAGTAGTTACAGT | 0.04 | 2 |
| | rs2307839-P | FAM-GGTGATTATGAGAGAACAA-BHQ1 | 0.1 | 3 |
| | rs112552066-F | Tag1-TGGAGGAAAAGTGGTAATGAGA | 0.06 | 4 |
| | rs112552066-R | Tag2-CAGCAGAAGGAACAGGA | 0.06 | 5 |
| | rs112552066-P | FAM- | 0.2 | 6 |
| | | ACTCAGAGAGACTACGAGCCAGCTTTAAGCAAC- | | |
| | | BHQ1 | | |
| | rs5858210-F | Tag1-CAGCCTCACTTTTGAACAC | 0.06 | 7 |
| | rs5858210-R | Tag2-GCTGACAGGGAGGAAAAC | 0.06 | 8 |
| | rs5858210-P | HEX-TCTCCAACAAACCAGAGT-BHQ1 | 0.2 | 9 |
| | rs66960151-F | Tag1-TGTCTGACTGCTGATTTGAT | 0.06 | 10 |
| | rs66960151-R | Tag2-GAGTGGCCATGTAGGAT | 0.06 | 11 |
| | rs66960151-P | HEX-AGAAGAGACGACAACGCTGTGAGGCTC- | 0.2 | 12 |
| | | BHQ1 | | |
| | rs68076527-F | Tag1-CAGAAATGAGATTCATTTGCTGGA | 0.03 | 13 |
| | rs68076527-R | Tag2-GTCTAGGCCACTTCCCTC | 0.03 | 14 |
| | rs68076527-P | ROX-TCTTTGAGTGTCAATTTC-BHQ2 | 0.2 | 15 |
| | rs5789826-F | Tag1-GCCCTGTTTCAAGTTATCTG | 0.03 | 16 |
| | rs5789826-R | Tag2-TTGGGCCAGTCTAGCAG | 0.03 | 17 |
| | rs5789826-P | ROX-TCCAGTGACTCTGCTATTATGGTAAGT- | 0.4 | 18 |
| | | BHQ2 | | |
| | rs34765837-F | Tag1-GCGTTAGTCAGTCTTACCCTAAAC | 0.03 | 19 |
| | rs34765837-R | Tag2-TACACGAGTTTCGTTCTTTGC | 0.03 | 20 |
| | rs34765837-P | ROX- | 0.2 | 21 |
| | | GCACATTCCGGGAGGGCGTTATGGGCACTG- | | |
| | | BHQ2 | | |
| | rs16363-F | Tag1-TAGAAGAACACAGTGGGGC | 0.05 | 22 |
| | rs16363-R | Tag2-GCTGGATTGAAGTGCATTTGA | 0.05 | 23 |
| | rs16363-P | CY5-CAAAACAAAACAGGATTCA-BHQ2 | 0.3 | 24 |
| | rs1610937-F | Tag1-GTTAGGAAGCCAAATAGGATGT | 0.05 | 25 |
| | rs1610937-R | Tag2-CACTTACACTAGAATGAGCA | 0.05 | 26 |
| | rs1610937-P | CY5- | 0.2 | 27 |
| | | CACGAGGAAGGAAGGGAAGACATGACCCA- | | |
| | | BHQ2 | | |
| | rs1611048-F | Tag1-TACAAGCACAAATGAACAAG | 0.03 | 28 |
| | rs1611048-R | Tag2-TTTACTGTAATTCCACTCCACT | 0.03 | 29 |
| | rs1611048-P | QUASAR705- | 0.3 | 30 |
| | | GGGCATTTGACTGACAGAGTAGGGGACAGTC | | |
| | | GGAGAGC-BHQ3 | | |
| | rs149809066-F | Tag1-CATTAGTTATGTCCACTATTCA | 0.03 | 31 |
| | rs149809066-R | Tag2-TCTTGCAATAACCCTCACAG | 0.03 | 32 |
| | rs149809066-P | QUASAR705-AGTAAGGAAAGTAATTATTTCA- | 0.2 | 33 |
| | | BHQ3 | | |
| | rs2307553-F | Tag1-GGTTACCAAGACCAGATGGA | 0.04 | 34 |
| | rs2307553-R | Tag2-GCACACATGCACATGAGT | 0.04 | 35 |
| | rs2307553-P | QUASAR705-GCTCTCTCTCAGTTGGGACTT- | 0.2 | 36 |
| | | BHQ3 | | |
| 2 | rs6424243-F | Tag1-CCACATCTCCTCCAGCA | 0.06 | 37 |
| | rs6424243-R | Tag2-CAAAGGGATGGGTTCCTC | 0.06 | 38 |
| | rs6424243-P | FAM-CGCAGCTACAAATGTACACTGCG- | 0.2 | 39 |
| | | BHQ1 | | |
| | rs12990278-F | Tag1-TAGGTGTGAACGAGCCTG | 0.06 | 40 |
| | rs12990278-R | Tag2-CCTGTTAGAGCTCCCAC | 0.06 | 41 |
| | rs12990278-P | FAM- | 0.2 | 42 |
| | | CGGTCCCCAGCCCTGTAGCCACGACCG- | | |
| | | BHQ1 | | |
| | rs2122080-F | Tag1-TAGTCTCAGTGGACTTTGGT | 0.06 | 43 |
| | rs2122080-R | Tag2-CAAACATCAAACAATTCAGCA | 0.06 | 44 |
| | rs2122080-P | HEX-ACCTGAGAATGTGGTTACTTGCAGGT- | 0.2 | 45 |
| | | BHQ1 | | |
| | rs98506667-F | Tag1-TCCCCACCCAGAAGAAAC | 0.03 | 46 |
| | rs98506667-R | Tag2-GGGAGGAGAAGGACTGATG | 0.03 | 47 |
| | rs98506667-P | HEX- | 0.5 | 48 |
| | | CCTCAGCTGTCCTCCCCACTTCCGTCACTGAGG- | | |
| | | BHQ1 | | |
| | rs774763-F | Tag1-CCCCAGTAATGGCAGATCA | 0.03 | 49 |
| | rs774763-R | Tag2-TGCCTTCCAGATATGCATTC | 0.03 | 50 |
| | rs774763-P | ROX-ACAGCAAGTCAATTCACTGT-BHQ2 | 0.2 | 51 |
| | rs10779650-F | Tag1-CTCCAGAATCAAGCTGTGT | 0.03 | 52 |
| | rs10779650-R | Tag2-TCATGTAGGAGTGCATTGT | 0.03 | 53 |
| | rs10779650-P | ROX-CCAGTAAGACAGCTGTACACTGGT- | 0.2 | 54 |
| | | BHQ2 | | |
| | rs4971514-F | Tag1-CGTATCATTCGGTTATCAAG | 0.05 | 55 |
| | rs4971514-R | Tag2-CCCATCTGAGCAAAGAACT | 0.05 | 56 |
| | rs4971514-P | ROX- | 0.3 | 57 |
| | | AATCGGCCGGATTTCCCTCCAGGTACCGAT- | | |
| | | BHQ2 | | |
| | rs711725-F | Tag1-TAATTTCTCTATGCTCATAGGTTCT | 0.05 | 58 |
| | rs711725-R | Tag2-TTCAAACCTCCTATTCCACAG | 0.05 | 59 |
| | rs711725-P | CY5-ACAGCACATGTAACATATGGAGTGCT- | 0.3 | 60 |
| | | BHQ2 | | |
| | rs2053911-F | Tag2-GCACAGGCAATTGAGAAGA | 0.03 | 61 |
| | rs2053911-R | Tag1-CTCCTTTAAAAGGGTCGGT | 0.03 | 62 |
| | rs2053911-P | CY5- | 0.3 | 63 |
| | | ACAGCCCATTTGTTTCTCCTGTCTTGAGGCTG- | | |
| | | BHQ2 | | |
| | rs9613776-F | Tag2-CCAAACTCCTGGATCATAAAACA | 0.03 | 64 |
| | rs9613776-R | Tag1-GGAATCAGGGATAATCTCTATCA | 0.03 | 65 |
| | rs9613776-P | Quasar 705-TCCAGGGTGCTTACACTG-BHQ3 | 0.2 | 66 |
| | rs7160304-F | Tag1-TCTACCGTCTAACCTGCAAG | 0.03 | 67 |
| | rs7160304-R | Tag2-ATCTACGCCTGAGGGACA | 0.03 | 68 |
| | rs7160304-P | Quasar705- | 0.2 | 69 |
| | | TGCTGCCTGAGTGATGATAAGTGTCAGCA- | | |
| | | BHQ3 | | |

| | | | |
|---|---|---|---|
| Tag2 | GTCGCAAGCACTCACGTAGAGA | 2.4 | 70 |
| Tag1 | TCGCAAGCACTCACGTAGAG | 0.3 | 71 |

The SNP typing system was performed in a 25-L-PCR reaction containing 1×PCR buffer (TAKARA, Beijing), 5.0 mM MgCl₂, 0.2 mM dNTPs, 1 U Taq DNA polymerase (TAKARA, Beijing), primers and probes listed in Table 2, and 5 μL of human genomic DNA or negative control (water). The PCR amplification program was done as follow: pre-denaturation at 95° C. for 5 min; 10 cycles of denaturation at 95° C. for 15 s, annealing at 65° C. to 56° C. for 15 s (1° C. decreased per cycle), extension at 76° C. for 20 s; 50 cycles of denaturation at 95° C. for 15 s, annealing at 55° C. for 15 s, extension at 76° C. for 20 s, then the melting curve analysis was carried out as follow: 95° C. for 1 min, 37° C. for 3 min, followed by a temperature increase from 40° C. to 85° C. at a heating rate of 0.04° C./s, and the fluorescence signals of FAM, HEX, ROX, CY5, and Quasar705 channels were collected. The instrument used in this experiment was SLAN 96 real-time fluorescent PCR instrument (Shanghai Hongshi Medical Technology Co., Ltd.). The typical results of SNP typing of the donor and recipient samples of the bone marrow transplant cases in this example were shown in FIG. 4.

4. Screening of Target SNP Sites

Target SNP site was obtained by comparing the genotypes of the corresponding SNP sites in the donor DNA sample and the recipient DNA sample, that was, the same SNP site in the donor DNA sample and the recipient DNA sample, at which the genotype of the SNP site of the donor sample was homozygous AA (or BB), and the genotype of the SNP site of the recipient sample was another homozygous BB (or AA); or the genotype of the SNP site of the donor sample was homozygous AA (or BB), and the genotype of the SNP site of the recipient sample was heterozygous AB. In this example, taking the bone marrow transplantation Case 1 sample group and Case 2 sample group as examples, the SNP typing results of the donor DNA samples and the recipient DNA samples were shown in Table 3 and FIG. 4. Wherein, there were 6 target SNP sites in the Case 1 sample group (i.e., rs2307839, rs16363, rs12990278, rs4971514, rs9613376, rs7160304), and two of the preferred target SNP sites (i.e., rs12990278, rs4971514) were selected and subjected to the quantitative analysis of their allele copy numbers by the digital PCR system, thereby determining their donor chimerism rates. There were 10 target SNP sites in the Case 2 sample group (i.e., rs2307839, rs66960151, rs68076527, rs5789826, rs1611048, rs149809066, rs12990278, rs2122080, rs774763, rs9613776), and the two preferred target SNP sites (i.e., rs5789826, rs2122080) were selected and subjected to the quantitative analysis of their allele copy numbers by the digital PCR system, thereby determining their donor chimerism rates.

TABLE 3
SNP typing results of bone marrow transplantation Case 1 sample group and
Case 2 sample group

| Sample name | Case 1 donor | Case 1 recipient | Case 2 donor | Case 2 recipient |
|---|---|---|---|---|
| SNP name | (black solid line) | (black dotted line) | (gray solid line) | (gray dotted line) |
| rs2307839 | GA/GA | GA/− | −/− | GA/− |
| rs112552066 | AGAG/− | −/− | AGAG/− | AGAG/− |
| rs5858210 | AG/AG | AG/AG | AG/AG | AG/AG |
| rs66960151 | TG/− | TG/TG | TG/TG | TG/− |
| rs68076527 | −/− | −/− | −/− | TT/− |
| rs5789826 | GTAA/− | GTAA/GTAA | GTAA/GTAA | GTAA/− |
| rs34765837 | −/− | −/− | ACAT/− | ACAT/ACAT |
| rs16363 | −/− | TGTTT/− | TGTTT/− | −/− |
| rs1610937 | −/− | −/− | AGGA/− | −/− |
| rs1611048 | TAAG/− | TAAG/− | TAAG/TAAG | TAAG/− |
| rs149809066 | CT/− | CT/CT | CT/CT | CT/− |
| rs2307553 | TGAC/− | TGAC/− | TGAC/− | TGAC/TGAC |
| rs6424243 | G/G | G/G | G/A | A/A |
| rs12990278 | T/T | C/T | T/T | C/T |
| rs2122080 | G/T | G/T | G/G | G/T |
| rs98506667 | G/G | G/G | G/C | G/C |
| rs774763 | G/G | G/G | G/G | C/C |
| rs10779650 | A/A | A/A | A/G | A/G |
| rs4971514 | G/G | G/C | G/G | G/G |
| rs711725 | T/A | A/A | T/A | T/A |
| rs2053911 | A/A | A/A | G/A | G/A |
| rs9613776 | G/G | A/G | G/G | A/G |
| rs7160304 | G/G | G/T | T/T | T/T |

5. Quantitative Detection of Genomic DNA Sample

Digital PCR quantitative analysis systems were established respectively according to the selected target SNP sites, each system contained a pair of primers and two fluorescent probes specific for different alleles of SNP. The primers and probes used in the quantitative analysis system for each SNP site were shown in Table 4. For the selected target SNP sites, the corresponding primer sets and probe sets in the digital PCR system were used to determine the ratio of each allele of the target SNP sites.

The instruments involved in Droplet digital PCR system contained Drop Marker instrument, Chip Reader instruments (Xinyi Biotechnology, Beijing) and Langji A300 amplification instrument (Langji Scientific Instrument Co., Ltd., Hangzhou). 30 μL of PCR reaction solution contained ddPCR general amplification reagent (Xinyi Biotechnology, Beijing), artificially synthesized sequence (Shanghai Bioengineering, Shanghai). Optionally, after pre-enrichment of the aforementioned cell-free DNA samples, the determination of SNP allele copy number was carried out by using the primer set and the probe set corresponding to the target SNP site in the digital PCR system, upstream and downstream primers had a concentration of 0.8 μmol/L, and the fluorescent probe had a concentration of 0.25 μmol/L. After the genomic DNA sample was added to PCR Master Mix, the Drop Marker instrument was used to prepare nanoliter-level droplets. The PCR amplification program is as follow: 95° C. pre-denaturation for 10 min, 40 cycles of denaturation at 94° C. for 30 s, annealing at 58° C. for 60 s, and incubation at 12° C. after amplification, with an overall temperature change rate of 1.5° C./s. After the PCR reaction, the Chip Reader instrument was used to quantitatively detect the droplets, and the reading system was used to download the sample detection data in Excel format, including the numbers of negative and positive droplets and the copy numbers of FAM and HEX fluorescence channels.

6. Calculation of Donor Chimerism Rate

The quantitative analysis model of donor chimerism rate was derived according to the biallelic characteristics of SNP molecular markers and the Hardy-Weinberg equilibrium law of genetic balance.

1) If for the target SNP site as selected, the donor SNP genotype was AA, the recipient SNP genotype was BB, the number of recipient allele B as determined by the digital PCR was N_{B}, and the number of donor allele A as determined by the digital PCR was N_{A}, then the percentage of the donor genomic DNA in the recipient's total genomic DNA was the donor chimerism rate:

$$donor chimerism rate = 1 - \frac{{N}_{B}}{{N}_{A} + {N}_{B}}$$

2) If for the target SNP site as selected, the donor SNP genotype was BB, the recipient SNP genotype was AA, the number of recipient allele A as determined by the digital PCR was N_{A}, and the number of donor allele B as determined by the digital PCR was N_{B}, then the percentage of the donor genomic DNA in the recipient's total genomic DNA was the donor chimerism rate:

$$donor chimerism rate = 1 - \frac{{N}_{A}}{{N}_{A} + {N}_{B}}$$

3) If for the target SNP site as selected, the donor SNP genotype was AA, the recipient SNP genotype was AB, the number of recipient allele B as determined by the digital PCR was N_{B}, and the number of donor allele A as determined by the digital PCR was N_{A}, then the percentage of the donor genomic DNA in the recipient's total genomic DNA was the donor chimerism rate:

$$donor chimerism rate = 1 - \frac{2 {N}_{B}}{{N}_{A} + {N}_{B}}$$

4) If for the target SNP site as selected, the donor SNP genotype was BB, the recipient SNP genotype was AB, the number of recipient allele A as determined by the digital PCR was N_{A}, and the number of donor allele B as determined by the digital PCR was N_{B}, then the percentage of the donor genomic DNA in the recipient's total genomic DNA was the donor chimerism rate:

$$donor chimerism rate = 1 - \frac{2 {N}_{A}}{{N}_{A} + {N}_{B}}$$

For the detection of multiple target SNP sites, firstly the donor chimerism rate was detected on the basis of each target SNP site, and then the average value thereof was calculated as the donor chimerism rate in the analysis report.

TABLE 4
Primers and probes used in the digital PCR quantitative analysis system

| | Sequence | | | SEQ ID |
|---|---|---|---|---|
| SNP name | name | Sequence (5′-3′) | Concentration | No. |
| rs2307839 | F | AGGTAATCTGAGGTGGCATC | 0.8 | 72 |
| | R | TGTAATTTCCTACCTAAGTAGTTACAGT | 0.8 | 73 |
| | WT-P | FAM-GGTGATTATGAGAGAACAACCTTC- | 0.25 | 74 |
| | | BHQ1 | | |
| | MT-P | HEX-GGTGATTATGAGAACAACCTTC-BHQ1 | 0.25 | 75 |
| rs112552066 | F | ATGGAAAATGTAATATTTCTGAATGAAAGA | 0.8 | 76 |
| | R | CCTTTCATCTAAATGCGTTGC | 0.8 | 77 |
| | WT-P | FAM-TTGAAACTCAGAGAGACTACGAG-BHQ1 | 0.25 | 78 |
| | MT-P | HEX-TGAAACTCAGACTACGAGCC-BHQ1 | 0.25 | 79 |
| rs5858210 | F | CGCTGGGTCATCTATTAACAC | 0.8 | 80 |
| | R | GAATGCCAGTATTCACAACAGT | 0.8 | 81 |
| | WT-P | FAM-ACAAACCAGAGTCTTCTTATGAAG- | 0.25 | 82 |
| | | BHQ1 | | |
| | MT-P | HEX-TCCAACAAACCAGTCTTCTT-BHQ1 | 0.25 | 83 |
| rs66960151 | F | TCCAATCCAGTGTTTCTTCTGA | 0.8 | 84 |
| | R | CACCCAGACAAGCCACC | 0.8 | 85 |
| | WT-P | FAM-GACAACGCTGTGAGGCTCT-BHQ1 | 0.25 | 86 |
| | MT-P | HEX-CGACAACGCTGAGGCTCT-BHQ1 | 0.25 | 87 |
| rs68076527 | F | AGAAATGAGATTCATTTGCTGGA | 0.8 | 88 |
| | R | GTCTAGGCCACTTCCCTC | 0.8 | 89 |
| | WT-P | FAM-CACCTCTTTGAGTGTCAATTTCC-BHQ1 | 0.25 | 90 |
| | MT-P | HEX-CACCTCTGAGTGTCAATTTCCC-BHQ1 | 0.25 | 91 |
| rs5789826 | F | AATTACACATCCCTCATTTATCCAG | 0.8 | 92 |
| | R | TGCAATTAAAATCTATTGAGCAATGG | 0.8 | 93 |
| | WT-P | FAM-CTGCTATTATGGTAAGTGTCGGA-BHQ1 | 0.25 | 94 |
| | MT-P | HEX-GCTATTATGGTGTCGGATTCA-BHQ1 | 0.25 | 95 |
| rs34765837 | F | CGTTAGTCAGTCTTACCCTAAAC | 0.8 | 96 |
| | R | ACACGAGTTTCGTTCTTTGC | 0.8 | 97 |
| | WT-P | FAM-CGGCACATTCCGGGAGG-BHQ1 | 0.25 | 98 |
| | MT-P | HEX-ACTGCCCGGCTCCGG-BHQ1 | 0.25 | 99 |
| rs16363 | F | AGAAGAACACAGTGGGGC | 0.8 | 100 |
| | R | GCTGGATTGAAGTGCATTTGA | 0.8 | 101 |
| | WT-P | FAM-GGACAACAAAACAAAACAGGATTC- | 0.25 | 102 |
| | | BHQ1 | | |
| | MT-P | HEX-GGGACAACAAAACAGGATTCA-BHQ1 | 0.25 | 103 |
| rs1610937 | F | CATTTAGGAAGCCAAATAGGATGTAC | 0.8 | 104 |
| | R | GTAAAAGTCTGCAGAAAATGGGT | 0.8 | 105 |
| | WT-P | FAM-ACGAGGAAGGAAGGGAAGA-BHQ1 | 0.25 | 106 |
| | MT-P | HEX-CACGAGGAAGGGAAGACAT-BHQ1 | 0.25 | 107 |
| rs1611048 | F | TGCATCCTTGCTGACGA | 0.8 | 108 |
| | R | AGCTTTTATTTCAGATACCTGTTGA | 0.8 | 109 |
| | WT-P | FAM-TGCAGTAACTACAAGTAAGGAAAGTA- | 0.25 | 110 |
| | | BHQ1 | | |
| | MT-P | HEX-TGCAGTAACTACAAGGAAAGTAATT- | 0.25 | 111 |
| | | BHQ1 | | |
| rs149809066 | F | CCCACTGATCATCTCCCAAA | 0.8 | 112 |
| | R | CACTATGGTGATTCCTAGTACCTT | 0.8 | 113 |
| | WT-P | FAM-GTGTTGCTCTCTCTCAGTTGG-BHQ1 | 0.25 | 114 |
| | MT-P | HEX-GTTGCTCTCTCAGTTGGG-BHQ1 | 0.25 | 115 |
| rs2307553 | F | GCATGCATTTCAAAGTTTATACCTG | 0.8 | 116 |
| | R | CAAGGAGAGCAATAAGTATGTATCG | 0.8 | 117 |
| | WT-P | FAM-CATTTGACTGACAGAGTAGGGG-BHQ1 | 0.25 | 118 |
| | MT-P | HEX-AGTGGGCATTTGACAGAGTA-BHQ1 | 0.25 | 119 |
| rs6424243 | F | AGCAGATCCTTGGTCAGT | 0.8 | 120 |
| | R | CAAAGGGATGGGTTCCTCT | 0.8 | 121 |
| | WT-P | FAM-GCAGCTACAAATGTACACT-BHQ1 | 0.25 | 122 |
| | MT-P | HEX-GCAGCTACAAATATACACT-BHQ1 | 0.25 | 123 |
| rs12990278 | F | TCCACCATAAATCTCAACTATTCG | 0.8 | 124 |
| | R | GCTCCCACAACCTTCCT | 0.8 | 125 |
| | WT-P | FAM-GTTGCCCTGGTCATGG-BHQ1 | 0.25 | 126 |
| | MT-P | HEX-GTTGCCCTGGTCGTGG-BHQ1 | 0.25 | 127 |
| rs2122080 | F | GCATTAGCTGAATCCTTTAAGAGA | 0.8 | 128 |
| | R | AATCCTTAAAAACAATGCAGCAG | 0.8 | 129 |
| | WT-P | FAM-CTGAGAATGTTGTTACTTGCAG-BHQ1 | 0.25 | 130 |
| | MT-P | HEX-CTGAGAATGTGGTTACTTGCAG-BHQ1 | 0.25 | 131 |
| rs9856667 | F | GAAACCTTGCCATCTCCAG | 0.8 | 132 |
| | R | GGCATCAGTGACGGAAGT | 0.8 | 133 |
| | WT-P | FAM-GTTCCCAGCTCTCCTCCC-BHQ1 | 0.25 | 134 |
| | MT-P | HEX-GTTCCCAGCTGTCCTCC-BHQ1 | 0.25 | 135 |
| rs774763 | F | TCTGCTCAGTGTGACAAGT | 0.8 | 136 |
| | R | GAGTGTGATTTGATTTTTATGCTTTTG | 0.8 | 137 |
| | WT-P | FAM-TGTGAATTGACTTGCTGAGGAA-BHQ1 | 0.25 | 138 |
| | MT-P | HEX-TGAATTGACTTGGTGAGGA-BHQ1 | 0.25 | 139 |
| rs10779650 | F | GTTACAGATATTCCCAGAGCA | 0.8 | 140 |
| | R | TTCTCCCAATTCTCAAAGCA | 0.8 | 141 |
| | WT-P | FAM-CAGTAAGGCAGCTGTACAC-BHQ1 | 0.25 | 142 |
| | MT-P | HEX-CAGTAAGACAGCTGTACACTG-BHQ1 | 0.25 | 143 |
| rs4971514 | F | CATTCGGTTATCAAGTATTACCCA | 0.8 | 144 |
| | R | CTTTCTGGCTCATGTCTGAC | 0.8 | 145 |
| | WT-P | FAM-GATTTCCCTGCAGGTACCT-BHQ1 | 0.25 | 146 |
| | MT-P | HEX-CGGATTTCCCTCCAGGTACC-BHQ1 | 0.25 | 147 |
| rs711725 | F | GTCTTTAAGGATGTTCTCTAAATTTTTGT | 0.8 | 148 |
| | R | ACCTCCTATTCCACAGAAGATTAT | 0.8 | 149 |
| | WT-P | FAM-AGCACATGTAACATATGGAG-BHQ1 | 0.25 | 150 |
| | MT-P | HEX-AGCACATGTAACATAAGGAG-BHQ1 | 0.25 | 151 |
| rs2053911 | F | ATGATCTGAACAGAGCTTCTGA | 0.8 | 152 |
| | R | GGTCTGAGTTCACCTCCTC | 0.8 | 153 |
| | WT-P | FAM-CTCAAGACAGGAGAAAC-BHQ1 | 0.25 | 154 |
| | MT-P | HEX-CCTCAAGACAAGAGAAACA-BHQ1 | 0.25 | 155 |
| rs9613776 | F | CCAAACTCCTGGATCATAAAACA | 0.8 | 156 |
| | R | CAGGAAAAGAGCTGGGTCA | 0.8 | 157 |
| | WT-P | FAM-TCCAGGGTGCTTACACT-BHQ1 | 0.25 | 158 |
| | MT-P | HEX-ATCCAGGGTGCTCACACT-BHQ1 | 0.25 | 159 |
| rs7160304 | F | ACAAGCTCTCTCATCCTACATC | 0.8 | 160 |
| | R | CCCTGAGTCTGTCTGATCTG | 0.8 | 161 |
| | WT-P | FAM-TGCCTGAGTGATGATAAGTG-BHQ1 | 0.25 | 162 |
| | MT-P | HEX-TGCCTTAGTGATGATAAGTG-BHQ1 | 0.25 | 163 |

7. Detection Results

The method of the present invention was used to measure the postoperative donor chimerism rate of 2 cases of bone marrow transplantation, blood samples were collected at 4 time points after transplantation, the detection results of donor chimerism rates at different time points of various recipients were shown in Table 5. It could be seen from the results in the table that Case 1 and Case 2 were in a recovery state after transplantation, and the donor chimerism rates at these time points were all greater than 9500.

TABLE 5
Detection results of postoperative donor chimerism
rates in 2 cases of bone marrow transplantation

| | |
|---|---|
| Case 1 | Case 2 |

| Days after | Donor chimerism | Days after | Donor chimerism |
|---|---|---|---|
| operation | rate | operation | rate |
| 14 | 95.21% | 14 | 99.88% |
| 28 | 99.87% | 28 | 99.90% |
| 35 | 99.88% | 35 | 99.88% |
| 44 | 99.95% | 44 | 99.70% |

### Example 3. Determination of Ratio of Donor-Derived Cell-Free DNA in Organ Transplantation (with Donor Information)

In this example, the determination of ratio of donor-derived cfDNA in the plasma and urine samples from one case of kidney transplantation patient was taken as an example, to monitor the organ damage of kidney transplantation Case 3, and the feasibility of using the method of the present invention to measure the dd-cfDNA ratio in organ transplantation and detection performance thereof were investigated.

The detection process of this example case was shown in FIG. 3. Taking 1 case of kidney transplant sample group as an example, the following two parts of samples needed to be collected: (1) the donor samples and recipient samples of kidney transplant patient before transplantation were collected and extracted, and used for SNP typing, and the principle of SNP typing was shown in FIG. 1; or the blood cell sediment, saliva, tissues other than transplanted organ, skin, etc. after transplantation were collected to serve as recipient samples; (2) the peripheral blood and urine samples at various time points during the period of monitoring the recipient after transplantation were collected, the plasma and urine supernatants were separated, and cfDNAs were extracted for quantification of target SNPs, the dd-cfDNA ratios after organ transplantation were detected, and the damage degree of organ after transplantation was evaluated.

The specific operation steps of the above detection process were as follows:

1. Collection of Kidney Transplant Sample Group

Case 3 sample group comprised donor peripheral blood samples and recipient peripheral blood samples before transplantation, and recipient samples (plasma and urine) at various time points after transplantation. The blood samples were collected using EDTA anticoagulant tubes (Zhejiang Gongdong Medical Technology Co., Ltd., Taizhou), and plasma separation was performed within 2 hours after collection according to the standard separation process (1600 g, centrifuged for 10 min; 16000 g, centrifuged for 10 min), and the plasma samples were stored at −80° C.; the urine samples were collected using urine collection cups (Zhejiang Gongdong Medical Technology Co., Ltd., Taizhou), and supernatants were taken within 6 hours according to the standard separation process (5000 g, centrifuged for 20 min), and the urine supernatant samples were stored at −80° C.

2. Extraction of Genomic DNA and Cell-Free DNA

Lab-Aid 824 nucleic acid extractor and supporting reagents for blood genomic DNA extraction (Xiamen Zeesan Biotech Co., Ltd., Xiamen) were used to extract the genomic DNA of each blood sample, and Nanodrop 2000 spectrophotometer (Thermo Fisher Scientific, USA) was used to determine the concentration and purity of genomic DNA. Apostle MiniMax™ high efficiency cell-free DNA enrichment and separation kit (Apostle, USA) was used to extract cell-free DNA in the plasma and urine samples, and Qubit 3.0 fluorometer (Thermo Fisher Scientific, USA) was used to determine the concentration of cell-free DNA.

3. SNP Typing

According to the selected SNP sites, corresponding primers and probes were designed, and the multiple asymmetric PCR typing system (the principle was shown in FIG. 1) was used for typing 23 SNPs in two PCR reaction systems at the same time. The primers and probe sequences used and concentrations thereof were shown in Table 2. The SNP typing system was consistent with Example 2. FIG. 5 and Table 6 showed the typical SNP typing results of kidney transplant donor and recipient samples in this case.

TABLE 6
SNP typing results of organ transplantation Case 3 sample group

| Sample name | Case 3 donor | Case 3 recipient |
|---|---|---|
| SNP name | (black solid line) | (black dotted line) |
| rs2307839 | GA/− | GA/− |
| rs112552066 | AGAG/AGAG | AGAG/− |
| rs5858210 | AG/− | AG/− |
| rs66960151 | TG/− | TG/− |
| rs68076527 | TT/− | TT/− |
| rs5789826 | −/− | GTAA/− |
| rs34765837 | ACAT/− | ACAT/− |
| rs16363 | TGTTT/TGTTT | TGTTT/− |
| rs1610937 | AGGA/AGGA | AGGA/− |
| rs1611048 | TAAG/− | TAAG/− |
| rs149809066 | CT/CT | CT/CT |
| rs2307553 | TGAC/− | TGAC/− |
| rs6424243 | G/A | G/A |
| rs12990278 | C/T | C/T |
| rs2122080 | G/T | T/T |
| rs98506667 | C/C | G/C |
| rs774763 | G/C | G/C |
| rs10779650 | A/G | A/A |
| rs4971514 | G/C | G/C |
| rs711725 | T/T | T/T |
| rs2053911 | A/A | A/A |
| rs9613776 | A/G | A/G |
| rs7160304 | G/T | G/G |

4. Screening of Target SNP Sites

Target SNP site was obtained by comparing the genotypes of the corresponding SNP sites of the donor genomic DNA and the recipient genomic DNA in the Case 3 sample group, that was, the same SNP site in the donor DNA sample and the recipient DNA sample, at which the SNP site genotype of the donor sample was homozygous AA (or BB), the SNP site genotype of the recipient sample was another homozygous BB (or AA); or the SNP site genotype of the donor sample was heterozygous AB, and the SNP site genotype of the recipient sample was homozygous AA (or BB). In this example, there were 3 target SNP sites in the Case 3 sample group (i.e., rs2122080, rs10779650, rs7160304), and these 3 target SNP sites were subjected to quantitative analysis of allele copy number by using the digital PCR system, thereby determining the ratio of donor-derived cell-free DNA.

5. Pre-Enrichment of Cell-Free DNA Samples

According to the SNP sites selected in Example 1, pre-enrichment primers were designed, and each SNP enrichment primer pair was consistent with the primer pair used in the SNP quantification system in the digital PCR, which were specifically shown in Table 4 of Example 1. The pre-enrichment system was a 50 μL PCR reaction system containing 1×PCR buffer (TAKARA, Beijing), 5.0 mM MgCl₂, 0.2 mM dNTPs, 2 U Taq DNA polymerase (TAKARA, Beijing), various primers listed in Table 4, and 1 to 10 ng of cell-free DNA. The PCR amplification program was as follow: pre-denaturation at 95° C. for 5 min; 10 cycles of denaturation at 95° C. for 20 s, annealing at 58° C. for 4 min, and extension at 72° C. for 2 min. The instrument used in this experiment was A300 amplification instrument (Langji Scientific Instrument Co., Ltd., Hangzhou).

6. Quantitative Detection of Cell-Free DNA Samples

According to the selected SNP sites in Example 1, digital PCR quantitative analysis systems were established, respectively. Each system comprised a pair of primers and two probes respectively specific to the SNP alleles. The primers and probes and amounts thereof used in each SNP site quantitative system were shown in Table 4. For the selected target SNP site, the ratio of each allele of the target SNP site was determined by using the corresponding primer set and probe set in the digital PCR system. The quantitative detection system for cell-free DNA sample was consistent with that described for the quantitative detection of genomic DNA sample in case 1.

7. Calculation of Donor-Derived Cell-Free DNA Ratio

The calculation method of Case 3 was as follows:

The quantitative analysis model of dd-cfDNA could be deduced according to the biallelic characteristics of SNP molecular markers and the Hardy-Weinberg equilibrium law of genetic balance.

1) If for the target SNP site as selected, the donor SNP genotype was AA, the recipient SNP genotype was BB, the number of donor allele A as determined by the digital PCR was N_{A}, and the number of recipient allele B as determined by the digital PCR was N_{B}, then the ratio of donor-derived cfDNA to the total cfDNA from recipient sample was:

$$d d - cf DNA \% = \frac{{N}_{A}}{{N}_{A} + {N}_{B}}$$

2) If for the target SNP site as selected, the donor SNP genotype was BB, the recipient SNP genotype was AA, the number of donor allele B as measured by the digital PCR was N_{B}, and the number of recipient allele A as determined by the digital PCR was N_{A}, then the ratio of donor-derived cfDNA to the total cfDNA from recipient sample was:

$$d d - cf D NA \% = \frac{{N}_{B}}{{N}_{A} + {N}_{B}}$$

3) If for the target SNP site as selected, the donor SNP genotype was AB, the recipient SNP genotype was AA, the number of donor allele B as measured by the digital PCR was NB, and the number of recipient allele A as determined by the digital PCR was N_{A}, then the ratio of donor-derived cfDNA to the total cfDNA from recipient sample was:

$$d d - cf DNA \% = \frac{2 {N}_{B}}{{N}_{A} + {N}_{B}}$$

4) If for the target SNP site as selected, the donor SNP genotype was AB, the recipient SNP genotype was BB, the number of donor allele A as determined by the digital PCR was N_{A}, and the number of recipient allele B as determined by the digital PCR was N_{B}, then the ratio of donor-derived cfDNA to the total cfDNA from recipient sample was:

$$d d - cf D NA \% = \frac{2 {N}_{A}}{{N}_{A} + {N}_{B}}$$

For the above detection of multiple target SNP sites, firstly the dd-cfDNA ratios were detected on the basis of these each target SNP sites, and then the average value was calculated as the dd-cfDNA ratio in the analysis report.

8. Analysis of Detection Results

The method of the present invention was used to determine the dd-cfDNA ratios after kidney transplantation, and for the blood and urine samples collected at seven time points after transplantation, the dd-cfDNA ratios of the samples collected at different time points of each recipient were shown in Table 7.

TABLE 7
Detection results of dd-cfDNA ratios after
kidney transplantation in Case 3

| Days after operation | Blood dd-cfDNA ratio | Urine dd-cfDNA ratio |
|---|---|---|
| 1 | 8.47% | 56.30% |
| 3 | 0.24% | 25.27% |
| 7 | 0.11% | 16.23% |
| 14 | 0.12% | 23.00% |
| 21 | 0.06% | 5.31% |
| 32 | 0.10% | 62.09% |
| 60 | 0.06% | 12.02% |

### Example 4. Screening Target SNP Sites by Using Urine Cell-Free DNA after Kidney Transplantation

Donor samples might not be collected during organ transplantation monitoring. In this example, the recipient sample in Example 3 was taken as an example to simulate the situation that donor sample could not be obtained, in which the urine cell-free DNA from the recipient after kidney transplantation was used as a SNP typing template, to investigate the feasibility of the method of the present application for screening target SNP sites. Based on the urine dd-cfDNA ratio in Example 3 and literature reports, the urine dd-cfDNA ratio ranged from 500 to 8000.

According to the SNP sites selected in Example 1, corresponding primers and probes were designed, and the multiple asymmetric PCR typing system (principle shown in FIG. 1) was used for typing 23 SNPs in 2 PCR reaction systems at the same time, and the sequences and concentrations of the primers and probes used were shown in Table 2. The SNP typing system was consistent with Example 2. FIG. 5 and Table 7 showed the typical SNP typing results of the recipient urine cell-free DNA samples after kidney transplantation, and the donor and recipient genomic DNA samples before kidney transplantation.

TABLE 7
SNP typing results of organ transplantation Case 3 sample group

| | | Case 3 recipient |
|---|---|---|
| Sample name | Case 3 recipient | urine sample 3 days after |
| SNP name | (black dotted line) | operation (gray solid line) |
| rs2307839 | GA/− | GA/− |
| rs112552066 | AGAG/− | AGAG/− |
| rs5858210 | AG/− | AG/− |
| rs66960151 | TG/− | TG/− |
| rs68076527 | TT/− | TT/− |
| rs5789826 | GTAA/− | GTAA/− |
| rs34765837 | ACAT/− | ACAT/− |
| rs16363 | TGTTT/− | TGTTT/− |
| rs1610937 | AGGA/− | AGGA/− |
| rs1611048 | TAAG/− | TAAG/− |
| rs149809066 | CT/CT | CT/CT |
| rs2307553 | TGAC/− | TGAC/− |
| rs6424243 | G/A | G/A |
| rs12990278 | C/T | C/T |
| rs2122080 | T/T | G/T |
| rs98506667 | G/C | G/C |
| rs774763 | G/C | G/C |
| rs10779650 | A/A | A/G |
| rs4971514 | G/C | G/C |
| rs711725 | T/T | T/T |
| rs2053911 | A/A | A/A |
| rs9613776 | A/G | A/G |
| rs7160304 | G/G | G/T |

Target SNP sites were screened by comparing the genotypes of the SNP sites corresponding to the urine cell-free DNA and the recipient genomic DNA in the Case 3 sample group on the 3^{rd} day after surgery, that was, the same SNP site as screened, at which on the 3^{rd} day, the urine cell-free DNA sample and the recipient genomic DNA sample had different alleles. In this example, there were 3 screened target SNP sites (i.e., rs2122080, rs10779650, rs7160304). The screening results were consistent with the target SNP sites screened by using the donor genomic DNA sample and the recipient genomic DNA in Example 3, indicating that the urine cell-free DNA after kidney transplantation could be used for the screening of target SNP sites when the donor samples could not be obtained.

According to the investigation results of Example 4, the cell-free DNA extracted from blood and urine samples as collected after organ transplantation (e.g., peripheral blood cfDNA on the first day after transplantation or urine cfDNA after kidney transplantation) contained some donor-derived cell-free DNA, and when the donor-derived cell-free DNA reached a certain ratio (e.g., 20% or more), the cfDNA could be directly genotyped by using the SNP typing system, and the target SNP site could be screened by comparing with the SNP typing results of the recipient's own genomic DNA.

### Example 5. Determination of Ratio of Donor Cell-Free DNA in Organ Transplantation (without Donor Information)

In this example, kidney transplant Case 4 and Case 5 sample groups were taken as examples to investigate the feasibility and detection performance of the method of the present invention for determining the dd-cfDNA ratio after organ transplantation where donor samples could not be obtained.

The operation steps were as follows:

1. Collection of Sample Groups of 2 Cases of Kidney Transplantation

The recipient samples (blood) before transplantation and the recipient samples (blood, urine) at various time points after transplantation in the Case 4 sample group, and the recipient samples (blood) before transplantation and the recipient samples (blood) at various time points after transplantation in the Case 5 sample group were collected. Wherein, the blood samples were collected using EDTA anticoagulant tubes (Zhejiang Gongdong Medical Technology Co., Ltd., Taizhou), plasma separation was performed within 2 hours after collection according to the standard separation procedure (1600 g, centrifuged for 10 min; 16000 g, centrifuged for 10 min), and the plasma samples were stored at −80° C.; the urine samples were collected using urine collection cups (Zhejiang Gongdong Medical Technology Co., Ltd., Taizhou), and supernatants were taken within 6 hours after collection according to the standard separation procedure (5000 g, centrifuged for 20 min), and the urine supernatant samples were stored at −80° C.

2. Extraction of Genomic DNA and Cell-Free DNA

Lab-Aid 824 nucleic acid extraction instrument and supporting reagents for blood extraction (Xiamen Zeesan Biotech Co., Ltd., Xiamen) were used to extract the genomic DNA of the above blood samples, and Nanodrop 2000 spectrophotometer (Thermo Fisher Scientific, USA) was used to determine the concentration and purity of genomic DNA. Apostle MiniMax™ high efficiency cell-free DNA enrichment and separation kit (Apostle, USA) was used to extract cell-free DNA from blood and urine samples, and the concentration of cell-free DNA was measured using Qubit 3.0 fluorometer (Thermo Fisher Scientific, USA).

3. SNP Typing

According to the selected SNP sites, corresponding primers and probes were designed, the multiple asymmetric PCR typing system (the principle was shown in FIG. 1) was used for typing 23 SNPs in two PCR reaction systems at the same time. The sequences and concentrations of the primers and probes used were shown in Table 2. The SNP typing system was consistent with Example 2. FIG. 6 and Table 8 showed the typical SNP typing results of the recipient urine samples after kidney transplantation and recipient blood samples before or after kidney transplantation in this case.

TABLE 8
SNP typing results of organ transplantation Case 4 and Case 5 sample groups

| | Case 4 urine | | | Case 5 |
|---|---|---|---|---|
| | sample after | Case 4 recipient | Case 5 donor | recipient |
| Sample name | operation | genomic DNA | (without | (gray |
| SNP name | (black solid line) | (black dotted line) | sample) | dotted line) |
| rs2307839 | GA/− | GA/− | | -/- |
| rs112552066 | AGAG/AGAG | AGAG/AGAG | | -/- |
| rs5858210 | AG/AG | −/− | | AG/AG |
| rs66960151 | TG/− | TG/− | | TG/TG |
| rs68076527 | TT/− | TT/− | | -/- |
| rs5789826 | GTAA/− | −/− | | GTAA/- |
| rs34765837 | ACAT/− | −/− | | -/- |
| rs16363 | TGTTT/− | TGTTT/TGTTT | | TGTTT/- |
| rs1610937 | AGGA/− | AGGA/AGGA | | AGGA/AGGA |
| rs1611048 | TAAG/− | TAAG/− | | TAAG/− |
| rs149809066 | CT/− | CT/CT | | CT/− |
| rs2307553 | TGAC/− | −/− | | −/− |
| rs6424243 | G/A | G/A | | G/A |
| rs12990278 | T/T | C/T | | C/T |
| rs2122080 | G/T | G/T | | G/T |
| rs98506667 | C/C | C/C | | C/C |
| rs774763 | C/C | G/C | | G/C |
| rs10779650 | A/A | A/A | | A/A |
| rs4971514 | G/C | G/C | | G/C |
| rs711725 | T/A | T/A | | T/A |
| rs2053911 | A/A | G/A | | G/A |
| rs9613776 | G/G | A/G | | A/A |
| rs7160304 | G/T | G/T | | G/T |

4. Screening of Target SNP Sites

Target SNP sites were obtained by comparing the genotypes of the corresponding SNP sites of the urine cfDNA after kidney transplantation and recipient blood genomic DNA before kidney transplantation in the Case 4 sample group, that was, the SNP site at which the genotype of the recipient was homozygous AA (or BB), and the recipient urine cfDNA sample and the recipient genomic DNA sample showed different alleles after transplantation. In this example, there were 6 target SNP sites screened in Case 4 (i.e., rs5858210, rs5789826, rs34765837, rs16363, rs1610937, rs149809066), among which 3 target SNP sites (i.e., rs5858210, rs149809066, rs1610937) were selected and subjected to quantitative analysis of copy number of alleles by the dPCR system, and the ratio of the donor-derived cell-free DNA was determined.

For Case 5 without donor sample, the SNP sites at which the recipient sample genotype was homozygous (e.g., AA or BB) were selected; and in Case 5, there were 11 SNP sites at which the recipient sample genotype was homozygous (i.e., rs2307839, rs112552066, rs5858210, rs66960151, rs68076527, rs34765837, rs1610937, rs2307533, rs98506667, rs10779650, rs9613776), among which 8 SNP sites were selected, and the blood cfDNA samples after operation in Case 5 were subjected to the quantitative analysis of allele copy number at the 8 SNP sites, which was used for subsequent determination of donor-derived cell-free DNA ratio.

5. Pre-Enrichment of Cell-Free DNA Samples

According to the SNP sites selected in Example 1, pre-enrichment primers were designed, and each SNP enrichment primer pair was consistent with the primer pair used in the SNP quantification system in the digital PCR, which were specifically described in Table 4 of Example 1. The pre-enrichment system was a 50 μL PCR reaction system containing 1×PCR buffer (TAKARA, Beijing), 5.0 mM MgCl₂, 0.2 mM dNTPs, 2 U Taq DNA polymerase (TAKARA, Beijing), primers and amounts thereof as shown in Table 4, and 1 to 10 ng of cell-free DNA. The PCR amplification program was as follow: pre-denaturation at 95° C. for 5 min; 10 cycles of denaturation at 95° C. for 20 s, annealing at 58° C. for 4 min, and extension at 72° C. for 2 min. The instrument used in this experiment was A300 amplification instrument (Langji Scientific Instruments Co., Ltd., Hangzhou).

6. Quantitative Detection of Cell-Free DNA Samples

According to the selected SNP sites in Example 1, digital PCR quantitative analysis systems were established, respectively. Each system comprised a pair of primers and two probes specific to the SNP alleles, respectively. The primers and probes and amount thereof used in each SNP site quantitative system were shown in Table 4. For the selected target SNP site, the allele ratios of the target SNP site were determined by using the corresponding primer set and probe set in the digital PCR system. The quantitative detection system of the cell-free DNA sample was consistent with the quantitative detection of the genomic DNA sample as described in Case 1.

7. Calculation of Donor Cell-Free DNA Ratio

7.1 Calculation Method for Case 4

After reading the digital PCR results of the target SNP sites, the absolute copy numbers of different alleles could be obtained, and the copy number ratios of donor-specific alleles could be divided into two categories by cluster analysis (K-means). There was a two-fold relationship for the values of these two categories, that was, a two-fold copy number relationship for heterozygous and homozygous. The chi-square test was performed using the data of the two categories after cluster analysis, so as to determine whether there was a significant difference in the two-fold relationship. Taking the blood cfDNA sample of Case 4 on the first day after surgery as an example, the specific allele ratios of the 3 target SNP sites determined by the digital PCR system were shown in Table 9. The corrected average of the 3 target SNP sites (rs5858210, rs149809066, rs1610937) was used as the dd-cfDNA ratio in the analysis report, which was 36.41%.

TABLE 9
Postoperative blood cfDNA sample analysis of organ
transplantation Case 4
Case 4

| SNP name | Specific allele ratio | Donor genotype |
|---|---|---|
| rs5858210 | 35.30% | Homozygous (target SNP) |
| rs149809066 | 36.81% | Homozygous (target SNP) |
| rs1610937 | 18.56% | Heterozygous (target SNP) |

7.2 Calculation Method for Case 5

It was known that the SNP genotype of the recipient of Case 5 was homozygous AA or BB, so that it could be considered that the alleles in the blood cfDNA sample of Case 5 after operation that were different from those of the recipient genomic DNA of Case 5 could be mostly from the donor, and a very small part thereof was caused by signal interference below the blank detection limit of the digital PCR. The SNP genotype of the donor might be heterozygous or homozygous, but its true genotype was unknown. After reading the digital PCR results, the absolute copy numbers of different alleles could be obtained, and the copy number ratios of donor-specific alleles could be divided into two categories by cluster analysis (K-means), and there was a two-fold relationship for the values of these two categories, that was, the two-fold copy number relationship between heterozygous and homozygous. The chi-square test was performed using the two data of the two categories after cluster analysis, so as to determine whether there was a significant difference in the two-fold relationship. Taking the blood cfDNA sample of Case 5 on the 2^{nd} day after operation as an example, the 8 SNP sites whose genotypes were homozygous in the recipient sample were selected for quantitative analysis by the digital PCR system, and the specific allele ratios as determined were shown in Table 10. The corrected average of the 4 target SNP sites (rs2307839, rs66960151, rs10779650, rs9613776) was used as the dd-cfDNA ratio in the analysis report, which was 3.76%.

TABLE 10
Postoperative blood cfDNA sample analysis of recipient
homozygous sites in organ transplant Case 5
Case 5

| SNP name | Specific allele ratio | Donor genotype |
|---|---|---|
| rs2307839 | 3.17% | Homozygous (target SNP) |
| rs112552066 | 0.02% | Homozygous (target SNP) |
| rs5858210 | 0.05% | Homozygous (target SNP) |
| rs66960151 | 4.05% | Homozygous (target SNP) |
| rs68076527 | 0.07% | Homozygous (target SNP) |
| rs34765837 | 0.01% | Homozygous (target SNP) |
| rs10779650 | 1.95% | Heterozygous (target SNP) |
| rs9613776 | 1.96% | Heterozygous (target SNP) |

8. Analysis of Detection Results

The method of the present invention was used to determine the postoperative dd-cfDNA ratios of 2 cases of organ transplantation, and blood samples were collected at 4 time points after transplantation. After the detection, the dd-cfDNA ratios of samples collected at different time points of each recipient were shown in Table 11.

TABLE 11
Detection results of dd-cfDNA ratios after operation
in 2 cases of organ transplantation

| | |
|---|---|
| Case 4 | Case 5 |

| Days after | Blood dd-cfDNA | Days after | Blood dd-cfDNA |
|---|---|---|---|
| operation | ratio | operation | ratio |
| 1 | 36.41% | 2 | 3.76% |
| 3 | 4.21% | 5 | 0.7% |
| 7 | 0.39% | 7 | 0.80% |
| 14 | 0.29% | 14 | 0.41% |
| 21 | 0.59% | 21 | 0.81% |

Although the specific models of the present invention have been described in detail, those skilled in the art will understand that: according to all the teachings that have been disclosed, various modifications and changes can be made to the details, and these changes are all within the protection scope of the present invention. The full scope of the present invention is given by the appended claims and any equivalents thereof.

What is claimed is:

1. A method for detecting a SNP site with different genotypes between a donor and a recipient, comprising the following steps:
(a) providing a first sample containing one or more target nucleic acids derived from the donor, and a second sample containing one or more target nucleic acids derived from the recipient, in which the target nucleic acids contain one or more candidate SNP sites, and,
providing a first universal primer and a second universal primer, and, for each candidate SNP site, providing at least one target-specific primer pair; wherein,
the first universal primer comprises a first universal sequence;
the second universal primer comprises a second universal sequence, the second universal sequence comprises the first universal sequence and additionally comprises at least one nucleotide at the 3′ end of the first universal sequence;
the target-specific primer pair is capable of performing amplification using the target nucleic acid as a template to generate a nucleic acid product containing the candidate SNP site, and the target-specific primer pair contains a forward primer and a reverse primer, wherein, the forward primer comprises the first universal sequence and a forward nucleotide sequence specific to the target nucleic acid, and the forward nucleotide sequence is located at the 3′ end of the first universal sequence; the reverse primer comprises the second universal sequence and a reverse nucleotide sequence specific to the target nucleic acid, and the reverse nucleotide sequence is located at the 3′ end of the second universal sequence; and, the second universal sequence is not completely complementary to a complementary sequence of the forward primer; and
(b) under a condition that allows nucleic acid amplification, amplifying the target nucleic acids in the first sample and the second sample, respectively, by using the first universal primer and the second universal primer and the target-specific primer pair, thereby obtaining amplification products respectively corresponding to the first sample and the second sample;
(c) performing melting curve analysis on the amplification products corresponding to the first sample and the second sample obtained in step (b);
(d) according to the result of the melting curve analysis of step (c), determining such an SNP site at which the first sample and the second sample have different genotypes;
preferably, in step (d) of the method, the genotypes of each candidate SNP site of the first sample and the second sample are determined according to the result of the melting curve analysis, thereby detecting an SNP site with different genotypes in the donor and the recipient;
preferably, the recipient has received or intends to receive or be transplanted with an organ, tissue or cell from the donor;
preferably, the recipient has received or intends to receive or be transplanted with an organ (e.g., kidney, heart, lung, liver, pancreas or any combination thereof) from the donor;
preferably, the recipient has or intends to receive or be transplanted with a hematopoietic stem cell (e.g., bone marrow hematopoietic stem cell, peripheral blood hematopoietic stem cell, umbilical cord blood hematopoietic stem cell or any combination thereof) or a hematopoietic stem cell-containing tissue or organ (e.g., bone marrow) from the donor;
preferably, the second sample is substantially free of nucleic acids from the donor;
preferably, the first sample is from the donor; for example, the first sample comprises a cell or tissue from the donor; for example, the first sample is selected from the group consisting of skin, saliva, urine, blood, hair, nail, or any combination thereof from the donor;
preferably, the second sample is from the recipient (e.g., the recipient who has or has not undergone transplantation); for example, the second sample comprises a cell or tissue from the recipient; for example, the second sample is selected from the group consisting of skin, saliva, urine, blood, hair, nail, or any combination thereof from the recipient;
preferably, in step (a), for each candidate SNP site, a detection probe is also provided, the detection probe comprises a nucleotide sequence specific to the target nucleic acid and is capable of performing annealing or hybridization to a region containing the candidate SNP site in the target nucleic acid, and the detection probe is labeled with a reporter group and a quencher group, wherein the reporter group is capable of emitting a signal, and the quencher group is capable of absorbing or quenching the signal emitted by the reporter group; and, the signal emitted by the detection probe when it is hybridized to its complementary sequence is different from the signal emitted when it is not hybridized to its complementary sequence;
and, in step (c), the amplification products corresponding to the first sample and the second sample obtained in step (b) are respectively subjected to melting curve analysis using the detection probe;
preferably, the first sample comprises DNA (e.g., genomic DNA);
preferably, the second sample comprises DNA (e.g., genomic DNA).

2. A method for detecting the presence or proportion of nucleic acids of a donor in a sample from a recipient who has undergone transplantation, wherein the method comprises the following steps:
(1) providing a nucleic acid-containing sample to be tested from the recipient who has been transplanted with a cell, tissue or organ from the donor;
(2) identifying one or more target SNP sites, wherein, at the target SNP site, the recipient has a first genotype comprising a first allele, and the donor has a second genotype comprising a second allele, wherein the first genotype is different from the second genotype and the first allele is different from the second allele;
(3) performing quantitative detection on the first allele and the second allele of each target SNP site in the sample to be tested, respectively; then, according to the results of the quantitative detection of the first allele and the second allele, determining the presence or proportion of the nucleic acids from the donor in the sample to be tested;
preferably, in step (2), the target SNP site can be identified by discriminating different alleles at a certain SNP site by a mechanism selected from the group consisting of: probe hybridization, primer extension, hybridization ligation and specific digestion;
preferably, in step (2), the target SNP site can be identified by a method selected from the group consisting of: sequencing method (e.g., first-generation sequencing method, pyrosequencing method, next-generation sequencing method), chip method (e.g., using solid-phase chip, liquid-phase chip capable of detecting SNP), qPCR-based assay (e.g., Tagman probe method), mass spectrometry (e.g., iPLEX™ Gold based on MassARRAY), chromatography (e.g., denaturing high performance liquid chromatography, dHPLC), electrophoresis (e.g., SNPshot method), detection method based on melting curve analysis;
preferably, in step (2), the target SNP site is identified by a detection method based on multiplex PCR combined with melting curve analysis;
preferably, the target SNP site is identified by the method described in claim 1;
preferably, in step (3), the first allele and the second allele of each target SNP site in the sample are quantitatively detected by digital PCR;
preferably, step (3) is carried out by the following scheme:
(I) selecting at least one (e.g., 1, 2, 3, or more) target SNP site from step (2), and, for each selected target SNP site, providing an amplification primer set and a probe set, wherein,
(I-1) the amplification primer set comprises at least one amplification primer (e.g., a pair of amplification primers or more amplification primers), which can specifically amplify a nucleic acid molecule containing the target SNP site under a condition that allows acid hybridization or annealing;
(I-2) the probe set comprises a first probe and a second probe; wherein,
(i) the first probe and the second probe are each independently labeled with a reporter group and a quencher group, wherein the reporter group can emit a signal, and the quencher group is capable of absorbing or quenching the signal emitted by the reporter group; and, the first probe and the second probe are respectively labeled with different reporter groups (e.g., fluorophores); and
(ii) the first probe is capable of hybridizing or annealing (preferably completely complementary) to a nucleic acid molecule containing the first allele of the target SNP site, and the second probe is capable of hybridizing or annealing (preferably completely complementary) to a nucleic acid molecule containing the second allele of the target SNP site; and, the first probe and the second probe are specific for different alleles;
(II) performing digital PCR on the sample from the recipient using the amplification primer set and the probe set to quantitatively detect the nucleic acid molecule having the first allele and the nucleic acid molecule having the second allele;
(III) according to the quantitative detection result of step (II), determining the presence or proportion of the nucleic acids from the donor in the sample to be tested;
preferably, the first probe specifically anneals or hybridizes to the nucleic acid molecule having the first allele during the digital PCR reaction; and the second probe specifically anneals or hybridizes to the nucleic acid molecule having the second allele during the digital PCR reaction;
preferably, the first probe does not anneal or hybridize to the nucleic acid molecule having the second allele during the digital PCR reaction; and/or, the second probe does not anneal or hybridize to the nucleic acid molecule having the first allele during the digital PCR reaction;
preferably, before step (3), the sample to be tested from the recipient is subjected to a pretreatment;
preferably, the pretreatment comprises extracting nucleic acids from the sample and/or enriching nucleic acids in the sample (e.g., by concentration and/or amplification).

3. The method according to claim 1 or 2, wherein the recipient has received or transplanted with a hematopoietic stem cell (e.g., bone marrow hematopoietic stem cell, peripheral blood hematopoietic stem cell, umbilical cord blood hematopoietic stem cell, or any combination thereof) or a hematopoietic stem cell-containing tissue or organ (e.g., bone marrow) from the donor;
preferably, the sample to be tested comprises blood (e.g., peripheral blood) or component thereof (e.g., blood cell, plasma, monocyte, granulocyte, T cell, or any combination thereof) from the recipient after transplantation;
preferably, the target SNP site is an SNP site at which the recipient has a first genotype comprising a homozygous first allele, and the donor has a second genotype comprising a homozygous second allele.

4. The method according to claim 1 or 2, wherein the recipient has received or transplanted with an organ (e.g., kidney, heart, lung, liver, pancreas, or any combination thereof) from the donor;
preferably, the recipient has received or transplanted with a kidney from the donor;
preferably, the sample to be tested comprises blood (e.g., peripheral blood) or urine (especially in the case of kidney transplantation) from the recipient after transplantation;
preferably, the target SNP site is an SNP site at which the donor has a first genotype comprising a homozygous first allele, and the recipient has a second genotype comprising a homozygous second allele.

5. The method according to any one of claims 1 to 4, wherein steps (a) to (b) of the method are carried out by a scheme comprising the following steps (I) to (VI):
(I) providing the first sample, the second sample, the first universal primer and the second universal primer, and the target-specific primer pair; and optionally, the detection probe;
(II) mixing the sample with the first universal primer, the second universal primer, the target-specific primer pair, a nucleic acid polymerase, and optionally, the detection probe;
(III) incubating the product of the previous step under a condition that allows nucleic acid denaturation;
(IV) incubating the product of the previous step under a condition that allows nucleic acid annealing or hybridization;
(V) incubating the product of the previous step under a condition that allows nucleic acid extension; and
(VI) optionally, repeating steps (III) to (V) once or more times;
preferably, the method has one or more technical features selected from the following:
(1) in step (III), incubating the product of step (II) at a temperature of 80 to 105° C., thereby denaturing the nucleic acid;
(2) in step (III), incubating the product of step (II) for 10 to 20 s, 20 to 40 s, 40 to 60 s, 1 to 2 min, or 2 to 5 min;
(3) in step (IV), incubating the product of step (III) at a temperature of 35 to 40° C., 40 to 45° C., 45 to 50° C., 50 to 55° C., 55 to 60° C., 60 to 65° C., or 65 to 70° C., thereby allowing the nucleic acid annealing or hybridization;
(4) in step (IV), incubating the product of step (III) for 10 to 20 s, 20 to 40 s, 40 to 60 s, 1 to 2 min, or 2 to 5 min;
(5) in step (V), incubating the product of step (IV) at a temperature of 35 to 40° C., 40 to 45° C., 45 to 50° C., 50 to 55° C., 55 to 60° C., 60 to 65° C., 65 to 70° C., 70 to 75° C., 75 to 80° C., 80 to 85° C., thereby allowing the nucleic acid extension;
(6) in step (V), incubating the product of step (IV) for 10 to 20 s, 20 to 40 s, 40 to 60 s, 1 to 2 min, 2 to 5 min, 5 to 10 min, 10 to 20 min or 20 to 30 min;
(7) performing steps (IV) and (V) at the same or different temperatures; and
(8) repeating steps (III) to (V) at least once, such as at least 2 times, at least 5 times, at least 10 times, at least 20 times, at least 30 times, at least 40 times, or at least 50 times; preferably, when repeating steps (III) to (V) once or more times, the conditions used in each cycle of steps (III) to (V) are independently the same or different.

6. The method according to any one of claims 2 to 5, wherein the primers of the amplification primer set each independently have one or more technical features selected from the following:
(1) the primers have a length of 15 to 20 nt, 20 to 30 nt, 30 to 40 nt, 40 to 50 nt, 50 to 60 nt, 60 to 70 nt, 70 to 80 nt, 80 to 90 nt, 90 nt to 100 nt, 100 to 110 nt, 110 to 120 nt, 120 to 130 nt, 130 to 140 nt, 140 to 150 nt;
(2) the primers or any constituent thereof comprise or consist of naturally occurring nucleotides (e.g., deoxyribonucleotides or ribonucleotides), modified nucleotides, non-natural nucleotides, or any combination;
(3) the amplification primer set comprises a primer pair having nucleotide sequences selected from the following or any combination thereof (e.g., any combination of 5 pairs, 10 pairs, 15 pairs, 20 pairs, 23 pairs): SEQ ID NOs: 72 and 73; 77 and 76; 80 and 81; 84 and 85;
88 and 89; 92 and 93; 96 and 97; 100 and 101; 104 and 105; 108 and 109; 112 and 113; 116 and 117; 120 and 121; 124 and 125; 128 and 129; 132 and 133; 136 and 137; 140 and 141; 144 and 145; 148 and 149; 152 and 153; 156 and 157; 160 and 161.

7. The method according to any one of claims 2 to 6, wherein the first probe and the second probe each independently have one or more features selected from the group consisting of:
(1) the first probe and the second probe each independently comprise or consist of naturally occurring nucleotides (e.g., deoxyribonucleotides or ribonucleotides), modified nucleotides, non-natural nucleotides (e.g., peptide nucleic acid (PNA) or locked nucleic acid), or any combination thereof,
(2) the first probe and the second probe each independently have a length of 15 to 20 nt, 20 to 30 nt, 30 to 40 nt, 40 to 50 nt, 50 to 60 nt, 60 to 70 nt, 70 to 80 nt, 80 to 90 nt, 90 to 100 nt, 100 to 200 nt, 200 to 300 nt, 300 to 400 nt, 400 to 500 nt, 500 to 600 nt, 600 to 700 nt, 700 to 800 nt, 800 to 900 nt, 900 to 1000 nt;
(3) the first probe and the second probe each independently have a 3′-OH end; or, the 3′-end of the probe is blocked; for example, the 3′-end of the probe is blocked by adding a chemical moiety (e.g., biotin or alkyl) to the 3′-OH of the last nucleotide of the probe, or by removing the 3′-OH of the last nucleotide of the probe, or replacing the last nucleotide with a dideoxynucleotide;
(4) the first probe and the second probe are each independently a self-quenching probe; for example, the probe is labeled with a reporter group at or upstream of its 5′ end and labeled with a quencher group at or downstream of its 3′ end, or labeled with a reporter group at or downstream of its 3′ end and labeled with a quencher group at or upstream of its 5′ end; preferably, the reporter group and the quencher group are separated by a distance of 10 to 80 nt or longer;
(5) the reporter group in the probe is a fluorophore (e.g., ALEX-350, FAM, VIC, TET, CAL Fluor Gold 540, JOE, HEX, CAL Fluor Orange 560, TAMRA, CAL Fluor Red 590, ROX, CAL Fluor Red 610, TEXAS RED, CAL Fluor Red 635, Quasar 670, CY3, CY5, CY5.5, Quasar 705); and, the quencher group is a molecule or group (e.g., DABCYL, BHQ (e.g., BHQ-1 or BHQ-2), ECLIPSE, and/or TAMRA) capable of absorbing/quenching the fluorescence;
(6) the first probe and the second probe each independently are linear or have a hairpin structure;
(7) the first probe and the second probe have different reporter groups; preferably, the first probe and the second probe are degradable by a nucleic acid polymerase (e.g., a DNA polymerase);
(8) the probe set comprises probes having nucleotide sequences selected from the following or any combination thereof (e.g., any combination of 5, 10, 20, 40, 60 probes): SEQ ID NOs: 73, 74, 78, 79, 82, 83, 86, 87, 90, 91, 94, 95, 98, 99, 102, 103, 106, 107, 110, 111, 114, 115, 118, 119, 122, 123, 126, 127, 130, 131, 134, 135, 138, 139, 142, 143, 146, 147, 150, 151, 154, 155, 158, 159, 162, 163.

8. A method for identifying an SNP site having a first genotype comprising a homozygous first allele in a recipient, which comprises the following steps:
(a) providing a fifth sample from the recipient, in which the fifth sample contains one or more target nucleic acids derived from the recipient and is substantially free of nucleic acids derived from a donor; the target nucleic acid comprises one or more candidate SNP sites, and,
providing a first universal primer and a second universal primer, and, for each candidate SNP site, providing at least one target-specific primer pair; wherein,
the first universal primer comprises a first universal sequence;
the second universal primer comprises a second universal sequence, the second universal sequence comprises the first universal sequence and additionally comprises at least one nucleotide at the 3′ end of the first universal sequence;
the target-specific primer pair is capable of performing amplification using the target nucleic acid as a template to generate a nucleic acid product containing the candidate SNP site, and the target-specific primer pair comprises a forward primer and a reverse primer, wherein, the forward primer comprises the first universal sequence and a forward nucleotide sequence specific to the target nucleic acid, and the forward nucleotide sequence is located at the 3′ end of the first universal sequence; the reverse primer comprises the second universal sequence and a reverse nucleotide sequence specific to the target nucleic acid, and the reverse nucleotide sequence is located at the 3′ end of the second universal sequence; and, the second universal sequence is not completely complementary to a complementary sequence of the forward primer; and
(b) under a condition that allows nucleic acid amplification, amplifying the target nucleic acid in the fifth sample by using the first universal primer and the second universal primer and the target-specific primer pair, respectively, thereby obtaining amplification products corresponding to the fifth sample;
(c) performing melting curve analysis on the amplification products corresponding to the fifth sample obtained in step (b);
(d) according to the results of the melting curve analysis of step (c), identifying such an SNP site at which the recipient has a first genotype comprising a homozygous first allele;
preferably, the fifth sample is from the recipient (e.g., the recipient who has or has not undergone transplantation); for example, the fifth sample comprises a cell or tissue from the recipient; for example, the fifth sample is selected from the group consisting of skin, saliva, urine, blood, hair, nail, or any combination thereof from the recipient;
preferably, in step (a), for each candidate SNP site, a detection probe is provided, the detection probe comprises a nucleotide sequence specific to the target nucleic acid and capable of annealing or hybridizing to a region of the target nucleic acid containing the candidate SNP site, and the detection probe is labeled with a reporter group and a quencher group, wherein the reporter group can emit a signal, and the quencher group is capable of absorbing or quenching the signal emitted by the reporter group; and, a signal emitted by the detection probe when it is hybridized to its complementary sequence is different from a signal emitted when it is not hybridized to its complementary sequence;
and, in step (c), the amplification products corresponding to the fifth sample obtained in step (b) are respectively subjected to melting curve analysis using the detection probe;
preferably, the fifth sample comprises DNA (e.g., genomic DNA).

9. A method for detecting the presence or proportion of nucleic acids of a donor in a sample from a recipient who has undergone transplantation, wherein the method comprises the following steps:
(1) providing a nucleic acid-containing sample to be tested from the recipient who has been transplanted with a cell, tissue or organ form the donor;
(2) identifying a plurality (e.g., at least 5, at least 6, at least 7, at least 8, at least 9, at least 10 or more) of such candidate SNP sites, in which the candidate SNP sites exhibit at least a first allele and a second allele in the species to which the recipient belongs, and, at the candidate SNP sites, the recipient has a first genotype comprising a homozygous first allele;
(3) performing quantitative detection of each allele of each candidate SNP site in the sample to be tested;
(4) according to the quantitative detection results of step (3), selecting such a target SNP site from the candidate SNP sites, at which the sample to be tested exhibits a signal of the first allele and a signal of the second allele;
(5) according to the results of quantitative detection of the first allele and the second allele of the target SNP site, determining the presence or proportion of the nucleic acids from the donor in the sample to be tested;
preferably, in step (2), the candidate SNP site can be identified by discriminating different alleles at a certain SNP site by a mechanism selected from the group consisting of: probe hybridization, primer extension, hybridization ligation and specific digestion;
preferably, in step (2), the candidate SNP site can be identified by a method selected from the group consisting of: sequencing method (e.g., first-generation sequencing method, pyrosequencing method, second-generation sequencing method), chip method (e.g., using solid-phase chip, liquid-phase chip capable of detecting SNP), qPCR-based assay (e.g., Taqman probe method), mass spectrometry (e.g., iPLEX™ Gold based on MassARRAY), chromatography (e.g., denaturing high performance liquid chromatography, dHPLC), electrophoresis (e.g., SNPshot method), detection method based on melting curve analysis;
preferably, in step (2), the candidate SNP site is identified by a detection method based on multiplex PCR combined with melting curve analysis;
preferably, the candidate SNP site is identified by the method described in claim 8;
preferably, in step (3), the alleles of the candidate SNP sites are quantitatively detected by digital PCR, respectively;
preferably, step (3) is carried out by the following scheme:
(I) selecting a plurality of (e.g., at least 5, at least 6, at least 7, at least 8, at least 9, at least 10, or more) candidate SNP sites from step (2), and, for each selected candidate SNP site, providing an amplification primer set and a probe set, wherein,
(I-1) the amplification primer set comprises at least one amplification primer (e.g., a pair of amplification primers or more amplification primers), which can specifically amplify a nucleic acid molecule containing the candidate SNP site under a condition that allows acid hybridization or annealing;
(I-2) the probe set comprises a first probe and a second probe; wherein,
(i) the first probe and the second probe are each independently labeled with a reporter group and a quencher group, wherein the reporter group can emit a signal, and the quencher group is capable of absorbing or quenching the signal emitted by the reporter group; and, the first probe and the second probe are respectively labeled with different reporter groups (e.g., fluorophores); and
(ii) the first probe is capable of hybridizing or annealing (preferably completely complementary) to a nucleic acid molecule containing the first allele of the candidate SNP site, and the second probe is capable of hybridizing or annealing (preferably completely complementary) to a nucleic acid molecule containing the second allele of the candidate SNP site; and, the first probe and the second probe are specific for different alleles;
(II) performing digital PCR on the sample to be tested from the recipient using the amplification primer set and the probe set to quantitatively detect the nucleic acid molecule having the first allele and the nucleic acid molecule having the second allele;
preferably, the first probe specifically anneals or hybridizes to the nucleic acid molecule having the first allele during the digital PCR reaction; and the second probe specifically anneals or hybridizes to the nucleic acid molecule having the second allele during the digital PCR reaction;
preferably, the first probe does not anneal or hybridize to the nucleic acid molecule having the second allele during the digital PCR reaction; and/or, the second probe does not anneal or hybridize to the nucleic acid molecule having the first allele during the digital PCR reaction;
preferably, in step (5), the quantitative detection results of the second allele of the plurality (e.g., at least 5, at least 6, at least 7, at least 8, at least 9, at least 10, or more) of target SNP sites are subjected to cluster analysis; then, according to the result of the cluster analysis, the genotype of the donor at each target SNP site is determined; then, according to the genotypes of the recipient and donor at each target SNP site, and the quantitative detection results of the first allele and the second allele in the sample to be tested, the presence or proportion of the nucleic acids of the donor in the sample to be tested from the recipient is determined;
preferably, before step (3), the sample to be tested from the recipient is subjected to a pretreatment;
preferably, the pretreatment comprises extracting nucleic acids from the sample and/or enriching nucleic acids in the sample (e.g., by concentration and/or amplification).

10. The method according to claim 8 or 9, wherein the recipient has received or transplanted with a hematopoietic stem cell (e.g., bone marrow hematopoietic stem cell, peripheral blood hematopoietic stem cell, umbilical cord blood hematopoietic stem cell or any combination thereof) or a hematopoietic stem cell-containing tissue or organ (e.g., the spinal cord) from the donor;
preferably, the sample to be tested comprises blood (e.g., peripheral blood) or component thereof (e.g., blood cell, plasma, monocyte, granulocyte, T cell, or any combination thereof) from the recipient after transplantation.

11. The method according to claim 8 or 9, wherein the recipient has received or transplanted with an organ (e.g., kidney, heart, lung, liver, pancreas, or any combination thereof) from the donor;
preferably, the recipient has received or transplanted a kidney from the donor;
preferably, the sample to be tested comprises blood (e.g., peripheral blood) or urine (especially in the case of kidney transplantation) from the recipient after transplantation.

12. The method according to any one of claims 8 to 11, wherein steps (a) to (b) of the method are carried out by a scheme comprising the following steps (I) to (VI):
(I) providing the fifth sample, the first universal primer and the second universal primer, and the target-specific primer pair; and optionally, the detection probe;
(II) mixing the fifth sample with the first universal primer, the second universal primer, the target-specific primer pair, a nucleic acid polymerase, and optionally, the detection probe;
(III) incubating the product of the previous step under a condition that allows nucleic acid denaturation;
(IV) incubating the product of the previous step under a condition that allows nucleic acid annealing or hybridization;
(V) incubating the product of the previous step under a condition that allows nucleic acid extension; and
(VI) optionally, repeating steps (III) to (V) once or more times;
preferably, the method has one or more technical features selected from the following:
(1) in step (III), incubating the product of step (II) at a temperature of 80 to 105° C., thereby denaturing the nucleic acid;
(2) in step (III), incubate the product of step (II) for 10 to 20 s, 20 to 40 s, 40 to 60 s, 1 to 2 min, or 2 to 5 min;
(3) in step (IV), incubating the product of step (III) at a temperature of 35 to 40° C., 40 to 45° C., 45 to 50° C., 50 to 55° C., 55 to 60° C., 60 to 65° C., or 65 to 70° C., thereby allowing the nucleic acid annealing or hybridization;
(4) in step (IV), incubating the product of step (III) for 10 to 20 s, 20 to 40 s, 40 to 60 s, 1 to 2 min, or 2 to 5 min;
(5) in step (V), incubating the product of step (IV) at a temperature of 35 to 40° C., 40 to 45° C., 45 to 50° C., 50 to 55° C., 55 to 60° C., 60 to 65° C., 65 to 70° C., 70 to 75° C., 75 to 80° C., 80 to 85° C., thereby allowing the nucleic acid extension;
(6) in step (V), incubating the product of step (IV) for 10 to 20 s, 20 to 40 s, 40 to 60 s, 1 to 2 min, 2 to 5 min, 5 to 10 min, 10 to 20 min, or 20 to 30 min;
(7) performing steps (IV) and (V) at the same or different temperatures; and
(8) repeating steps (III) to (V) at least once, such as at least 2 times, at least 5 times, at least 10 times, at least 20 times, at least 30 times, at least 40 times, or at least 50 times;
preferably, when repeating steps (III) to (V) once or more times, the conditions used in each cycle of steps (III) to (V) are independently the same or different.

13. The method according to any one of claims 9 to 12, wherein the primers of the amplification primer set each independently have one or more technical features selected from the following:
(1) the primers have a length of 15 to 20 nt, 20 to 30 nt, 30 to 40 nt, 40 to 50 nt, 50 to 60 nt, 60 to 70 nt, 70 to 80 nt, 80 to 90 nt, 90 nt to 100 nt, 100 to 110 nt, 110 to 120 nt, 120 to 130 nt, 130 to 140 nt, 140 to 150 nt;
(2) the primer or any constituent thereof comprises or consists of naturally occurring nucleotides (e.g., deoxyribonucleotides or ribonucleotides), modified nucleotides, non-natural nucleotides, or any combination thereof,
(3) the amplification primer set comprises a primer pair having nucleotide sequences selected from the following or any combination thereof (e.g., any combination of 5 pairs, 10 pairs, 15 pairs, 20 pairs, 23 pairs): SEQ ID NOs: 72 and 73; 77 and 76; 80 and 81; 84 and 85; 88 and 89; 92 and 93; 96 and 97; 100 and 101; 104 and 105; 108 and 109; 112 and 113; 116 and 117; 120 and 121; 124 and 125; 128 and 129; 132 and 133; 136 and 137; 140 and 141; 144 and 145; 148 and 149; 152 and 153; 156 and 157; 160 and 161.

14. The method according to any one of claims 9 to 13, wherein the first probe and the second probe each independently have one or more features selected from the group consisting of:
(1) the first probe and the second probe each independently comprise or consist of naturally occurring nucleotides (e.g., deoxyribonucleotides or ribonucleotides), modified nucleotides, non-natural nucleotides (e.g., peptide nucleic acid (PNA) or locked nucleic acid), or any combination thereof,
(2) the first probe and the second probe each independently have a length of 15 to 20 nt, 20 to 30 nt, 30 to 40 nt, 40 to 50 nt, 50 to 60 nt, 60 to 70 nt, 70 to 80 nt, 80 to 90 nt, 90 to 100 nt, 100 to 200 nt, 200 to 300 nt, 300 to 400 nt, 400 to 500 nt, 500 to 600 nt, 600 to 700 nt, 700 to 800 nt, 800 to 900 nt, 900 to 1000 nt;
(3) the first probe and the second probe each independently have a 3′-OH end; or, the 3′-end of the probe is blocked; for example, the 3′-end of the probe is blocked by adding a chemical moiety (e.g., biotin or alkyl) to the 3′-OH of the last nucleotide of the probe, or by removing the 3′-OH of the last nucleotide of the probe, or replacing the last nucleotide with a dideoxynucleotide;
(4) the first probe and the second probe are each independently a self-quenching probe; for example, the probe is labeled with a reporter group at or upstream of its 5′ end and labeled with a quencher group at or downstream of its 3′ end, or labeled with a reporter group at or downstream of its 3′ end and labeled with a quencher group at or upstream of its 5′ end; preferably, the reporter group and quencher group are separated by a distance of 10 to 80 nt or longer;
(5) the reporter group in the probe is a fluorophore (e.g., ALEX-350, FAM, VIC, TET, CAL Fluor Gold 540, JOE, HEX, CAL Fluor Orange 560, TAMRA, CAL Fluor Red 590, ROX, CAL Fluor Red 610, TEXAS RED, CAL Fluor Red 635, Quasar 670, CY3, CY5, CY5.5, Quasar 705); and, the quencher group is a molecule or group (e.g., DABCYL, BHQ (e.g., BHQ-1 or BHQ-2), ECLIPSE, and/or TAMRA) capable of absorbing/quenching the fluorescence;
(6) the first probe and the second probe each independently are linear or have a hairpin structure;
(7) the first probe and the second probe have different reporter groups; preferably, the first probe and the second probe are degradable by a nucleic acid polymerase (e.g., a DNA polymerase);
(8) the probe set comprises probes having nucleotide sequences selected from the following or any combination thereof (e.g., any combination of 5, 10, 20, 40, 60): SEQ ID NOs: 73, 74, 78, 79, 82, 83, 86, 87, 90, 91, 94, 95, 98, 99, 102, 103, 106, 107, 110, 111, 114, 115, 118, 119, 122, 123, 126, 127, 130, 131, 134, 135, 138, 139, 142, 143, 146, 147, 150, 151, 154, 155, 158, 159, 162, 163.

15. A method for detecting an SNP site with different genotypes between a donor and a recipient, comprising the following steps:
(a) providing a third sample from the recipient and a fourth sample from the recipient who has undergone transplantation, wherein the third sample contains one or more target nucleic acids derived from the recipient, and substantially does not contain nucleic acids derived from the donor; the fourth sample contains one or more target nucleic acids derived from the donor, and the target nucleic acid comprises one or more candidate SNP sites, and,
providing a first universal primer and a second universal primer, and, for each candidate SNP site, providing at least one target-specific primer pair; wherein,
the first universal primer comprises a first universal sequence;
the second universal primer comprises a second universal sequence, the second universal sequence comprises the first universal sequence and additionally comprises at least one nucleotide at the 3′ end of the first universal sequence;
the target-specific primer pair is capable of performing amplification using the target nucleic acid as a template to generate a nucleic acid product containing the candidate SNP site, and the target-specific primer pair comprises a forward primer and a reverse primer, wherein, the forward primer comprises the first universal sequence and a forward nucleotide sequence specific to the target nucleic acid, and the forward nucleotide sequence is located at the 3′ end of the first universal sequence; the reverse primer comprises the second universal sequence and a reverse nucleotide sequence specific to the target nucleic acid, and the reverse nucleotide sequence is located at the 3′ end of the second universal sequence; and, the second universal sequence is not completely complementary to a complementary sequence of the forward primer; and
(b) under a condition that allows nucleic acid amplification, amplifying the target nucleic acids in the third sample and the fourth sample by using the first universal primer and the second universal primer and the target-specific primer pair, respectively, thereby obtaining amplification products respectively corresponding to the third sample and the fourth sample;
(c) performing melting curve analysis on the amplification products corresponding to the third sample and the fourth sample obtained in step (b);
(d) according to the result of the melting curve analysis of step (c), determining such a SNP site at which the third sample only exhibits a first allele, and the fourth sample exhibits at least a second allele (e.g., exhibiting the first and second alleles); in which the SNP site is an SNP site with different genotypes between the donor and the recipient;
preferably, in the step (d) of the method, the genotypes of each candidate SNP site of the third sample and the fourth sample are determined according to the results of the melting curve analysis, so as to determine such an SNP site at which the third sample exhibits only the first allele, and the fourth sample exhibits both the first and second alleles;
preferably, the third sample is from the recipient (e.g., the recipient who has or has not undergone transplantation); for example, the third sample comprises a cell or tissue from the recipient; for example, the third sample is selected from the group consisting of skin, saliva, urine, blood, hair, nail, or any combination thereof from the recipient;
preferably, in the fourth sample, the amount of nucleic acids from the donor accounts for at least 20%, such as at least 25%, at least 30%, at least 35%, at least 40%, at least 50% or higher of the amount of total nucleic acids in the fourth sample;
preferably, the recipient has received or transplanted with an organ, tissue or cell from the donor;
for example, the recipient has received or transplanted with an organ (e.g., kidney, heart, lung, liver, pancreas, or any combination thereof) from the donor; preferably, the fourth sample comprises blood (e.g., peripheral blood) or urine (especially in the case of kidney transplantation) from the recipient who has undergone transplantation; preferably, the fourth sample comprises blood (e.g., peripheral blood) or urine (especially in the case of kidney transplantation) from the recipient who has undergone transplantation for no more than 5 days (e.g., no more than 3 days, 2 days or 1 day);
for example, the recipient has received or transplanted with a hematopoietic stem cell (e.g., bone marrow hematopoietic stem cell, peripheral blood hematopoietic stem cell, umbilical cord blood hematopoietic stem cell) or a hematopoietic stem cell-containing tissue or organ (e.g., bone marrow) from the donor; preferably, the fourth sample comprises blood (e.g., peripheral blood) or component thereof (e.g., blood cell) from the recipient who has undergone transplantation; preferably, the fourth sample comprises blood (e.g., peripheral blood) or component thereof (e.g., blood cell) from the recipient who has undergone transplantation for at least 5 days (e.g., at least 10 days, at least 15 days, at least 20 days, at least 30 days);
preferably, in step (a), for each candidate SNP site, a detection probe is also provided, the detection probe comprises a nucleotide sequence specific to the target nucleic acid and is capable of performing annealing or hybridization to a region containing the candidate SNP site in the target nucleic acid, and the detection probe is labeled with a reporter group and a quencher group, wherein the reporter group can emit a signal, and the quencher group is capable of absorbing or quenching the signal emitted by the reporter group; and, a signal emitted by the detection probe when it is hybridized to its complementary sequence is different from a signal emitted when it is not hybridized to its complementary sequence;
and, in step (c), the amplification products corresponding to the third sample and the fourth sample obtained in step (b) are respectively subjected to melting curve analysis using the detection probe;
preferably, the third sample comprises DNA (e.g., genomic DNA);
preferably, the fourth sample comprises DNA (e.g., genomic DNA).

16. A method for detecting the presence or proportion of nucleic acids of a donor in a sample from a recipient who has undergone transplantation, wherein the method comprises the following steps:
(1) providing a nucleic acid-containing sample to be tested from the recipient who has been transplanted with a cell, tissue or organ from the donor;
(2) identifying a plurality (e.g., at least 5, at least 6, at least 7, at least 8, at least 9, at least 10 or more) of target SNP sites, wherein, at the target SNP sites, the recipient has a first genotype comprising a homozygous first allele, and the donor has a second genotype comprising a second allele, wherein the first genotype is different from the second genotype, and the first allele is different from the second allele;
(3) performing quantitative detection on the first allele and the second allele of each target SNP site in the sample to be tested;
(4) according to the results of quantitative detection of the first allele and the second allele of the target SNP site, determining the presence or proportion of the nucleic acids from the donor in the sample to be tested;
preferably, in step (2), the target SNP site can be identified by discriminating different alleles at a certain SNP site by a mechanism selected from the group consisting of: probe hybridization, primer extension, hybridization ligation and specific digestion;
preferably, in step (2), the target SNP site can be identified by a method selected from the group consisting of: sequencing method (e.g., first-generation sequencing method, pyrosequencing method, second-generation sequencing method), chip method (e.g., using solid-phase chip, liquid-phase chip capable of detecting SNP), qPCR-based assay (e.g., Taqman probe method), mass spectrometry (e.g., iPLEX™ Gold based on MassARRAY), chromatography (e.g., denaturing high performance liquid chromatography, dHPLC), electrophoresis (e.g., SNPshot method), detection method based on melting curve analysis;
preferably, in step (2), the target SNP site is identified by a detection method based on multiplex PCR combined with melting curve analysis;
preferably, the target SNP site is identified by the method described in claim 15;
preferably, in step (3), the first allele and the second allele of each target SNP site in the sample are quantitatively detected by digital PCR;
preferably, step (3) is carried out by the following scheme:
(I) for each target SNP site, providing an amplification primer set and a probe set, wherein,
(I-1) the amplification primer set comprises at least one amplification primer (e.g., a pair of amplification primers or more amplification primers), which can specifically amplify a nucleic acid molecule containing the target SNP site under a condition that allows acid hybridization or annealing;
(I-2) the probe set comprises a first probe and a second probe; wherein,
(i) the first probe and the second probe are each independently labeled with a reporter group and a quencher group, wherein the reporter group can emit a signal, and the quencher group is capable of absorbing or quenching the signal emitted by the reporter group; and, the first probe and the second probe are respectively labeled with different reporter groups (e.g., fluorophores); and
(ii) the first probe is capable of hybridizing or annealing (preferably completely complementary) to a nucleic acid molecule containing the first allele of the target SNP site, and the second probe is capable of hybridizing or annealing (preferably completely complementary) to a nucleic acid molecule containing the second allele of the target SNP site; and, the first probe and the second probe are specific for different alleles;
(II) performing digital PCR on the sample to be tested using the amplification primer set and the probe set to quantitatively detect the nucleic acid molecule having the first allele and the nucleic acid molecule having the second allele;
preferably, the first probe specifically anneals or hybridizes to the nucleic acid molecule having the first allele during the digital PCR reaction; and the second probe specifically anneals or hybridizes to the nucleic acid molecule having the second allele during the digital PCR reaction;
preferably, the first probe does not anneal or hybridize to the nucleic acid molecule having the second allele during the digital PCR reaction; and/or, the second probe does not anneal or hybridize to the nucleic acid molecule having the first allele during the digital PCR reaction;
preferably, in step (4), the quantitative detection results of the second allele of the plurality (e.g., at least 5, at least 6, at least 7, at least 8, at least 9, at least 10, or more) of target SNP sites are subjected to cluster analysis; then, according to the result of the cluster analysis, the genotype of the donor at each target SNP site is determined; then, according to the genotypes of the recipient and donor at each target SNP site, and the quantitative detection results of the first allele and the second allele in the sample to be tested, the presence or proportion of the nucleic acid of the donor in the sample to be tested from the recipient is determined;
preferably, before step (3), the sample to be tested from the recipient is subjected to a pretreatment;
preferably, the pretreatment comprises extracting nucleic acids from the sample and/or enriching nucleic acids in the sample (e.g., by concentration and/or amplification).

17. The method according to claim 15 or 16, wherein the recipient has received or transplanted with a hematopoietic stem cell (e.g., bone marrow hematopoietic stem cell, peripheral blood hematopoietic stem cell, umbilical cord blood hematopoietic stem cell or any combination thereof) or a hematopoietic stem cell-containing tissue or organ (e.g., the spinal cord) from the donor;
preferably, the sample to be tested comprises blood (e.g., peripheral blood) or component thereof (e.g., blood cell, plasma, monocyte, granulocyte, T cell, or any combination thereof) from the recipient after transplantation.

18. The method according to claim 15 or 16, wherein the recipient has received or transplanted with an organ (e.g., kidney, heart, lung, liver, pancreas, or any combination thereof) from the donor;
preferably, the recipient has received or transplanted with a kidney from the donor;
preferably, the sample to be tested comprises blood (e.g., peripheral blood) or urine (especially in the case of kidney transplantation) from the recipient after transplantation.

19. The method according to any one of claims 15 to 18, wherein steps (a) to (b) of the method are carried out by a scheme comprising the following steps (I) to (VI):
(I) providing the third sample and the fourth sample, the first universal primer and the second universal primer, and the target-specific primer pair; and optionally, the detection probe;
(II) mixing the sample with the first universal primer, the second universal primer, the target-specific primer pair, a nucleic acid polymerase, and optionally, the detection probe;
(III) incubating the product of the previous step under a condition that allows nucleic acid denaturation;
(IV) incubating the product of the previous step under a condition that allows nucleic acid annealing or hybridization;
(V) incubating the product of the previous step under a condition that allows nucleic acid extension; and
(VI) optionally, repeating steps (III) to (V) once or more times;
preferably, the method has one or more technical features selected from the following:
(1) in step (III), incubating the product of step (II) at a temperature of 80 to 105° C., thereby denaturing the nucleic acid;
(2) in step (III), incubating the product of step (II) for 10 to 20 s, 20 to 40 s, 40 to 60 s, 1 to 2 min, or 2 to 5 min;
(3) in step (IV), incubating the product of step (III) at a temperature of 35 to 40° C., 40 to 45° C., 45 to 50° C., 50 to 55° C., 55 to 60° C., 60 to 65° C., or 65 to 70° C., thereby allowing the nucleic acid annealing or hybridization;
(4) in step (IV), incubating the product of step (III) for 10 to 20 s, 20 to 40 s, 40 to 60 s, 1 to 2 min, or 2 to 5 min;
(5) in step (V), incubating the product of step (IV) at a temperature of 35 to 40° C., 40 to 45° C., 45 to 50° C., 50 to 55° C., 55 to 60° C., 60 to 65° C., 65 to 70° C., 70 to 75° C., 75 to 80° C., 80 to 85° C., thereby allowing the nucleic acid extension;
(6) in step (V), incubating the product of step (IV) for 10 to 20 s, 20 to 40 s, 40 to 60 s, 1 to 2 min, 2 to 5 min, 5 to 10 min, 10 to 20 min or 20 to 30 min;
(7) performing steps (IV) and (V) at the same or different temperatures; and
(8) repeating steps (III) to (V) at least once, such as at least 2 times, at least 5 times, at least 10 times, at least 20 times, at least 30 times, at least 40 times, or at least 50 times;
preferably, when repeating steps (III) to (V) once or more times, the conditions used in each cycle of steps (III) to (V) are independently the same or different.

20. The method according to any one of claims 16 to 19, wherein the primers of the amplification primer set each independently have one or more technical features selected from the following:
(1) the primers have a length of 15 to 20 nt, 20 to 30 nt, 30 to 40 nt, 40 to 50 nt, 50 to 60 nt, 60 to 70 nt, 70 to 80 nt, 80 to 90 nt, 90 nt to 100 nt, 100 to 110 nt, 110 to 120 nt, 120 to 130 nt, 130 to 140 nt, 140 to 150 nt;
(2) the primers or any constituent thereof comprise or consist of naturally occurring nucleotides (e.g., deoxyribonucleotides or ribonucleotides), modified nucleotides, non-natural nucleotides, or any combination thereof,
(3) the amplification primer set comprises a primer pair having nucleotide sequences selected from the following or any combination thereof (e.g., any combination of 5 pairs, 10 pairs, 15 pairs, 20 pairs, 23 pairs): SEQ ID NOs: 72 and 73; 77 and 76; 80 and 81; 84 and 85; 88 and 89; 92 and 93; 96 and 97; 100 and 101; 104 and 105; 108 and 109; 112 and 113; 116 and 117; 120 and 121; 124 and 125; 128 and 129; 132 and 133; 136 and 137; 140 and 141; 144 and 145; 148 and 149; 152 and 153; 156 and 157; 160 and 161.

21. The method according to any one of claims 16 to 20, wherein the first probe and the second probe each independently have one or more features selected from the group consisting of:
(1) the first probe and the second probe each independently comprise or consist of naturally occurring nucleotides (e.g., deoxyribonucleotides or ribonucleotides), modified nucleotides, non-natural nucleotides (e.g., peptide nucleic acid (PNA) or locked nucleic acid), or any combination thereof,
(2) the first probe and the second probe each independently have a length of 15 to 20 nt, 20 to 30 nt, 30 to 40 nt, 40 to 50 nt, 50 to 60 nt, 60 to 70 nt, 70 to 80 nt, 80 to 90 nt, 90 to 100 nt, 100 to 200 nt, 200 to 300 nt, 300 to 400 nt, 400 to 500 nt, 500 to 600 nt, 600 to 700 nt, 700 to 800 nt, 800 to 900 nt, 900 to 1000 nt;
(3) the first probe and the second probe each independently have a 3′-OH end; or, the 3′-end of the probe is blocked; for example, the 3′-end of the probe is blocked by adding a chemical moiety (e.g., biotin or alkyl) to the 3′-OH of the last nucleotide of the probe, or by removing the 3′-OH of the last nucleotide of the probe, or replacing the last nucleotide with a dideoxynucleotide;
(4) the first probe and the second probe are each independently a self-quenching probe; for example, the probe is labeled with a reporter group at or upstream of its 5′ end and labeled with a quencher group at or downstream of its 3′ end, or labeled with a reporter group at or downstream of its 3′ end and labeled with a quencher group at or upstream of its 5′ end; preferably, the reporter group and quencher group are separated by a distance of 10 to 80 nt or longer;
(5) the reporter group in the probe is a fluorophore (e.g., ALEX-350, FAM, VIC, TET, CAL Fluor Gold 540, JOE, HEX, CAL Fluor Orange 560, TAMRA, CAL Fluor Red 590, ROX, CAL Fluor Red 610, TEXAS RED, CAL Fluor Red 635, Quasar 670, CY3, CY5, CY5.5, Quasar 705); and, the quencher group is a molecule or group (e.g., DABCYL, BHQ (e.g., BHQ-1 or BHQ-2), ECLIPSE, and/or TAMRA) capable of absorbing/quenching the fluorescence;
(6) the first probe and the second probe each independently are linear or have a hairpin structure;
(7) the first probe and the second probe have different reporter groups; preferably, the first probe and the second probe are degradable by a nucleic acid polymerase (e.g., a DNA polymerase);
(8) the probe set comprises probes having nucleotide sequences selected from the following or any combination thereof (e.g., any combination of 5, 10, 20, 40, 60): SEQ ID NOs: 73, 74, 78, 79, 82, 83, 86, 87, 90, 91, 94, 95, 98, 99, 102, 103, 106, 107, 110, 111, 114, 115, 118, 119, 122, 123, 126, 127, 130, 131, 134, 135, 138, 139, 142, 143, 146, 147, 150, 151, 154, 155, 158, 159, 162, 163.

22. The method according to any one of claims 1 to 21, wherein the candidate SNP site has one or more features selected from the following:
(1) the candidate SNP has a Fst of less than 0.3 (e.g., less than 0.2, less than 0.1, less than 0.05, less than 0.01) between different human races;
(2) the candidate SNP site is located on different chromosomes;
(3) the candidate SNP site has an allele frequency between 0.2 and 0.8 (e.g., between 0.3 and 0.7, between 0.4 and 0.6);
preferably, the candidate SNP site has one or more features selected from the following:
(1) the candidate SNP site has a Fst of less than 0.01 between different human races;
(2) the candidate SNP site is located on different chromosomes;
(3) the candidate SNP site has an allele frequency between 0.3 and 0.7;
preferably, the candidate SNP site is an SNP site with a biallelic polymorphism;
preferably, the candidate SNP site is an SNP site in the human genome; for example, the target nucleic acid comprises a human genome SNP site selected from the group consisting of: rs16363, rs1610937, rs5789826, rs1611048, rs2307533, rs112552066, rs5858210, rs2307839, rs149809066, rs66960151, rs34765837, rs68076527, rs10779650, rs4971514, rs6424243, rs12990278, rs2122080, rs98506667, rs774763, rs711725, rs2053911, rs9613776, rs7160304, and any combination of the aforementioned SNP sites (e.g., any combination of 5, 10, 15, 20, 23 of the aforementioned SNP sites);
preferably, the target nucleic acid in the sample comprises the following human genome SNP sites: rs16363, rs1610937, rs5789826, rs1611048, rs2307533, rs112552066, rs5858210, rs2307839, rs149809066, rs66960151, rs34765837, rs68076527, rs10779650, rs4971514, rs6424243, rs12990278, rs2122080, rs98506667, rs774763, rs711725, rs2053911, rs9613776 and rs7160304.

23. The method according to any one of claims 1 to 22, wherein the method has one or more technical features selected from the group consisting of:
(1) in step (b), the sample is mixed with the first universal primer, the second universal primer and the target-specific primer pair, and a nucleic acid polymerase, and subjected to nucleic acid amplification (e.g., PCR reaction), then, the detection probe is added to the product of step (b), and the melting curve analysis is carried out; or, in step (b), the sample is mixed with the first universal primer, the second universal primers, the target-specific primer pair, the detection probe, and a nucleic acid polymerase, and subjected to nucleic acid amplification (e.g., PCR reaction), and then, the melting curve analysis is carried out;
(2) the detection probe comprises or consists of naturally occurring nucleotides (e.g., deoxyribonucleotides or ribonucleotides), modified nucleotides, non-natural nucleotides (e.g., peptide nucleic acid (PNA) or locked nucleic acid), or any combination thereof,
(3) the detection probe has a length of 15 to 20 nt, 20 to 30 nt, 30 to 40 nt, 40 to 50 nt, 50 to 60 nt, 60 to 70 nt, 70 to 80 nt, 80 to 90 nt, 90 to 100 nt, 100 to 200 nt, 200 to 300 nt, 300 to 400 nt, 400 to 500 nt, 500 to 600 nt, 600 to 700 nt, 700 to 800 nt, 800 to 900 nt, 900 to 1000 nt;
(4) the detection probe has a 3′-OH end; or, the 3′-end of the detection probe is blocked; for example, the 3′-end of the detection probe is blocked by adding a chemical moiety (e.g., biotin or alkyl) to the 3′-OH of the last nucleotide of the detection probe, or removing the 3′-OH of the last nucleotide of the detection probe, or replacing the last nucleotide with a dideoxynucleotide;
(5) the detection probe is a self-quenching probe; for example, the detection probe is labeled with a reporter group at or upstream of its 5′ end and labeled with a quencher group at or downstream of its 3′ end, or labeled with a reporter group at or downstream of its 3′ end and labeled with a quencher group at or upstream of its 5′ end; preferably, the reporter group and the quencher group are separated by a distance of 10 to 80 nt or longer;
(6) the reporter group in the detection probe is a fluorophore (e.g., ALEX-350, FAM, VIC, TET, CAL Fluor Gold 540, JOE, HEX, CAL Fluor Orange 560, TAMRA, CAL Fluor Red 590, ROX, CAL Fluor Red 610, TEXAS RED, CAL Fluor Red 635, Quasar 670, CY3, CY5, CY5.5, Quasar 705); and, the quencher group is a molecule or group (e.g., DABCYL, BHQ (e.g., BHQ-1 or BHQ-2), ECLIPSE, and/or TAMRA) capable of absorbing/quenching the fluorescence;
(7) the detection probe has no resistance to nuclease activity, or has resistance to nuclease activity (e.g., 5′ nuclease activity, such as 5′ to 3′ exonuclease activity); for example, the backbone of the detection probe does not contain a nuclease-resistant modification, or contains a nuclease-resistant modification, such as thiophosphoester bond, alkylphosphotriester bond, arylphosphotriester bond, alkylphosphonate ester bond, arylphosphonate ester bond, hydrogenated phosphate ester bond, alkylaminophosphate ester bond, arylaminophosphate ester bond, 2′-O-aminopropyl modification, 2′-O-alkyl modification, 2′-O-allyl modification, 2′-O-butyl modification, and 1-(4′-thio-PD-ribofuranosyl) modification;
(8) the detection probe is linear or has a hairpin structure;
(9) the detection probes each independently have the same or different reporter groups; preferably, the detection probes have the same reporter group, and the product of step (b) is subjected to melting curve analysis, and then the presence of the target nucleic acid is determined according to the melting peak in the melting curve; or, the detection probes have different reporter groups, and the product of step (b) is subjected to melting curve analysis, and then the presence of the target nucleic acid is determined according to the signal type of the reporter group and the melting peak in the melting curve;
(10) in step (c), the product of step (b) is gradually heated or cooled, and a signal from reporter group on each detection probe is monitored in real time, so that the curve of signal intensity of each reporting group as a function of temperature is obtained; then, the curve is derived to obtain a melting curve of the product of step (b);
(11) the genotype of each SNP site is determined according to the melting peak (melting point) in the melting curve;
(12) the detection probe comprises a detection probe having nucleotide sequence selected from the following or any combination thereof (e.g., any combination of 5, 10, 15, 20, 23): SEQ ID NOs: 3, 6, 9, 12, 15, 18, 21, 24, 27, 30, 33, 36, 39, 42, 45, 48, 51, 54, 57, 60, 63, 66 and 69.

24. The method according to any one of claims 1 to 23, wherein the method has one or more technical features selected from the group consisting of:
(1) in step (a) of the method, 1 to 5, 5 to 10, 10 to 15, 15 to 20 or more target-specific primer pairs are provided;
(2) in step (b) of the method, the first universal primer and the second universal primer have a working concentration higher than that of the forward primer and the reverse primer; for example, the first universal primer and the second universal primer have a working concentration 1 to 5 times, 5 to 10 times, 10 to 15 times, 15 to 20 times, 20 to 50 times or more times higher than that of the forward primer and the reverse primer;
(3) in step (b) of the method, the first universal primer and the second universal primer have the same working concentration; or, the first universal primer has a working concentration lower than that of the second universal primer;
(4) in step (b) of the method, the forward primer and the reverse primer have the same or different working concentrations;
(5) the sample or target nucleic acid comprises mRNA, and the sample is subjected to a reverse transcription reaction prior to performing step (b) of the method; and
(6) in step (b) of the method, a nucleic acid polymerase (especially a template-dependent nucleic acid polymerase) is used for nucleic acid amplification; preferably, the nucleic acid polymerase is a DNA polymerase, such as a thermostable DNA polymerase; preferably, the thermostable DNA polymerase is obtained from, Thermus aquaticus (Taq), Thermus thermophiles (Tth), Thermus filiformis, Thermis flavus, Thermococcus literalis, Thermus antranildanii, Thermus caldophllus, Thermus chliarophilus, Thermus flavus, Thermus igniterrae, Thermus lacteus, Thermus oshimai, Thermus ruber, Thermus rubens, Thermus scotoductus, Thermus silvanus, Thermus thermophllus, Thermotoga maritima, Thermotoga neapolitana, Thermosipho africanus, Thermococcus litoralis, Thermococcus barossi, Thermococcus gorgonarius, Thermotoga maritima, Thermotoga neapolitana, Thermosiphoa fricanus, Pyrococcus woesei, Pyrococcus horikoshii, Pyrococcus abyssi, Pyrodictium occultum, Aquifex pyrophilus and Aquifex aeolieus; preferably, the DNA polymerase is a Taq polymerase.

25. The method according to any one of claims 1 to 24, wherein the method has one or more technical features selected from the group consisting of:
(1) the first universal primer consists of the first universal sequence, or comprises the first universal sequence and an additional sequence, and the additional sequence is located at the 5′ end of the first universal sequence; preferably, the additional sequence comprises 1 to 5, 5 to 10, 10 to 15, 15 to 20 or more nucleotides;
(2) the first universal sequence is located at or constitutes the 3′ portion of the first universal primer;
(3) the first universal primer has a length of 5 to 15 nt, 15 to 20 nt, 20 to 30 nt, 30 to 40 nt, or 40 to 50 nt;
(4) the first universal primer or any constituent thereof comprises or consists of naturally occurring nucleotides (e.g., deoxyribonucleotides or ribonucleotides), modified nucleotides, non-natural nucleotides, or any combination thereof,
(5) the second universal primer consists of the second universal sequence, or comprises the second universal sequence and an additional sequence, and the additional sequence is located at the 5′ end of the second universal sequence; preferably, the additional sequence comprises 1 to 5, 5 to 10, 10 to 15, 15 to 20 or more nucleotides;
(6) the second universal sequence is located at or constitutes the 3′ portion of the second universal primer;
(7) the second universal sequence comprises the first universal sequence and additionally comprises 1 to 5, 5 to 10, 10 to 15, 15 to 20 or more nucleotides at the 3′ end of the first universal sequence;
(8) the second universal primer has a length of 8 to 15 nt, 15 to 20 nt, 20 to 30 nt, 30 to 40 nt, or 40 to 50 nt; and
(9) the second universal primer or any constituent thereof comprises or consists of naturally occurring nucleotides (e.g., deoxyribonucleotides or ribonucleotides), modified nucleotides, non-natural nucleotides, or any combination thereof.

26. The method according to any one of claims 1 to 25, wherein the method has one or more technical features selected from the group consisting of:
(1) in the forward primer, the forward nucleotide sequence is directly ligated to the 3′ end of the first universal sequence, or ligated to the 3′ end of the first universal sequence through a nucleotide linker; preferably, the nucleotide linker comprises 1 to 5, 5 to 10, 10 to 15, 15 to 20 or more nucleotides;
(2) the forward primer also comprises an additional sequence, which is located at the 5′ end of the first universal sequence; preferably, the additional sequence comprises 1 to 5, 5 to 10, 10 to 15, 15 to 20 or more nucleotides;
(3) the forward primer comprises or consists of the first universal sequence and the forward nucleotide sequence from 5′ to 3′; or, comprises or consists of the first universal sequence and the nucleotide linker and the forward nucleotide sequence from 5′ to 3′; or, comprises or consists of the additional sequence, the first universal sequence and the forward nucleotide sequence from 5′ to 3′; or, comprises or consists of the additional sequence, the first universal sequence, the nucleotide linker and the forward nucleotide sequence from 5′ to 3′;
(4) the forward nucleotide sequence is located at or constitutes the 3′ portion of the forward primer;
(5) the forward nucleotide sequence has a length of 10 to 20 nt, 20 to 30 nt, 30 to 40 nt, 40 to 50 nt, 50 to 60 nt, 60 to 70 nt, 70 to 80 nt, 80 nt to 90 nt, 90 to 100 nt;
(6) the forward primer has a length of 15 to 20 nt, 20 to 30 nt, 30 to 40 nt, 40 to 50 nt, 50 to 60 nt, 60 to 70 nt, 70 to 80 nt, 80 to 90 nt, 90 to 100 nt, 100 to 110 nt, 110 to 120 nt, 120 to 130 nt, 130 to 140 nt, 140 to 150 nt;
(7) the forward primer or any constituent thereof comprises or consists of naturally occurring nucleotides (e.g., deoxyribonucleotides or ribonucleotides), modified nucleotides, non-natural nucleotides, or any combination thereof,
(8) in the reverse primer, the reverse nucleotide sequence is directly ligated to the 3′ end of the second universal sequence, or the reverse nucleotide sequence is ligated to the 3′ end of the second universal sequence through a nucleotide linker; preferably, the nucleotide linker comprises 1 to 5, 5 to 10, 10 to 15, 15 to 20 or more nucleotides;
(9) the reverse primer further comprises an additional sequence located at the 5′ end of the second universal sequence; preferably, the additional sequence comprises 1 to 5, 5 to 10, 10 to 15, 15 to 20 or more nucleotides;
(10) the reverse primer comprises or consists of the second universal sequence and the reverse nucleotide sequence from 5′ to 3′; or, comprises or consists of the second universal sequence, the nucleotide linker and the reverse nucleotide sequence from 5′ to 3′; or, comprises or consists of the additional sequence, the second universal sequence and the reverse nucleotide sequence from 5′ to 3′; or, comprises or consists of the additional sequence, the second universal sequence, the nucleotide linker and the reverse nucleotide sequence from 5′ to 3′;
(11) the reverse nucleotide sequence is located at or constitutes the 3′ portion of the reverse primer;
(12) the reverse nucleotide sequence has a length of 10 to 20 nt, 20 to 30 nt, 30 to 40 nt, 40 to 50 nt, 50 to 60 nt, 60 to 70 nt, 70 to 80 nt, 80 nt to 90 nt, 90 to 100 nt;
(13) the reverse primer has a length of 15 to 20 nt, 20 to 30 nt, 30 to 40 nt, 40 to 50 nt, 50 to 60 nt, 60 to 70 nt, 70 to 80 nt, 80 to 90 nt, 90 to 100 nt, 100 to 110 nt, 110 to 120 nt, 120 to 130 nt, 130 to 140 nt, 140 to 150 nt;
(14) the reverse primer or any constituent thereof comprises or consists of naturally occurring nucleotides (e.g., deoxyribonucleotides or ribonucleotides), modified nucleotides, non-natural nucleotides, or any combination thereof, and
(15) the second universal sequence is not completely complementary to a complementary sequence of the forward primer; for example, at least one nucleotide, such as 1 to 5, 5 to 10, 10 to 15, 15 to 20 or more nucleotides, located at the 3′ end in the second universal sequence is not complementary to a complementary sequence of the forward primer;
preferably, the sequence of the first universal primer is set forth in SEQ ID NO: 71;
preferably, the sequence of the second universal primer is set forth in SEQ ID NO: 70;
preferably, the target-specific primer pair comprises a primer pair having nucleotide sequences selected from the following or any combination thereof (e.g., any combination of 5 pairs, 10 pairs, 15 pairs, 20 pairs, 23 pairs): SEQ ID NOs: 1 and 2; 4 and 5; 7 and 8; 10 and 11; 13 and 14; 16 and 17; 19 and 20; 22 and 23; 25 and 26; 28 and 29; 31 and 32; 34 and 35; 37 and 38; 40 and 41; 43 and 44; 46 and 47; 49 and 50; 52 and 53; 55 and 56; 58 and 59; 61 and 62; 64 and 65; 67 and 68.

27. A kit, which comprises an identification primer set capable of asymmetrically amplifying a target nucleic acid containing a candidate SNP site;
preferably, the identification primer set comprises: a first universal primer and a second universal primer, and, for each candidate SNP site, at least one target-specific primer pair is provided, wherein,
the first universal primer comprises a first universal sequence;
the second universal primer comprises a second universal sequence, the second universal sequence comprises the first universal sequence and additionally comprises at least one nucleotide at the 3′ end of the first universal sequence;
the target-specific primer pair is capable of performing amplification by using the target nucleic acid as a template to generate a nucleic acid product containing the candidate SNP site, and the target-specific primer pair comprises a forward primer and a reverse primer, wherein, the forward primer comprises the first universal sequence and a forward nucleotide sequence specific to the target nucleic acid, and the forward nucleotide sequence is located at the 3′ end of the first universal sequence; the reverse primer comprises the second universal sequence and a reverse nucleotide sequence specific to the target nucleic acid, and the reverse nucleotide sequence is located at the 3′ end of the second universal sequence; and, the second universal sequence is not completely complementary to a complementary sequence of the forward primer;
preferably, the kit further comprises one or more detection probes capable of detecting the candidate SNP site, the detection probe comprises a nucleotide sequence specific to the target nucleic acid and is capable of performing annealing or hybridization to a region containing the candidate SNP site in the target nucleic acid, and is labeled with a reporter group and a quencher group, wherein the reporter group is capable of emitting a signal, and the quencher group is capable of absorbing or quenching the signal emitted by the reporter group; and a signal emitted by the detection probe when it is hybridized to its complementary sequence is different from a signal emitted when it is not hybridized to its complementary sequence;
preferably, the candidate SNP site has one or more features selected from the following:
(1) the candidate SNP has a Fst of less than 0.3 (e.g., less than 0.2, less than 0.1, less than 0.05, less than 0.01) between different human races;
(2) the candidate SNP site is located on different chromosomes;
(3) the candidate SNP site has an allele frequency between 0.2 and 0.8 (e.g., between 0.3 and 0.7, between 0.4 and 0.6);
preferably, the candidate SNP site has one or more features selected from the following:
(1) the candidate SNP site has a Fst of less than 0.01 between different human races;
(2) the candidate SNP site is located on different chromosomes;
(3) the candidate SNP site has an allele frequency between 0.3 and 0.7;
preferably, the candidate SNP site is an SNP site with a biallelic polymorphism;
preferably, the candidate SNP site is an SNP site in the human genome; for example, the target nucleic acid comprises a human genome SNP site selected from the group consisting of: rs16363, rs1610937, rs5789826, rs1611048, rs2307533, rs112552066, rs5858210, rs2307839, rs149809066, rs66960151, rs34765837, rs68076527, rs10779650, rs4971514, rs6424243, rs12990278, rs2122080, rs98506667, rs774763, rs711725, rs2053911, rs9613776, rs7160304, and any combination of the aforementioned SNP sites (e.g., any combination of 5, 10, 15, 20, 23 of the aforementioned SNP sites);
preferably, the target nucleic acid in the sample comprises the following human genome SNP sites: rs16363, rs1610937, rs5789826, rs1611048, rs2307533, rs112552066, rs5858210, rs2307839, rs149809066, rs66960151, rs34765837, rs68076527, rs10779650, rs4971514, rs6424243, rs12990278, rs2122080, rs98506667, rs774763, rs711725, rs2053911, rs9613776 and rs7160304;
preferably, the detection probe comprises a detection probe having a nucleotide sequence selected from the following or any combination thereof (e.g., any combination of 5, 10, 15, 20, 23): SEQ ID NO: 3, 6, 9, 12, 15, 18, 21, 24, 27, 30, 33, 36, 39, 42, 45, 48, 51, 54, 57, 60, 63, 66 and 69;
preferably, the sequence of the first universal primer is set forth in SEQ ID NO: 71;
preferably, the sequence of the second universal primer is set forth in SEQ ID NO: 70;
preferably, the target-specific primer pair comprises a primer pair having nucleotide sequences selected from the following or any combination thereof (e.g., any combination of 5 pairs, 10 pairs, 15 pairs, 20 pairs, 23 pairs): SEQ ID NOs: 1 and 2; 4 and 5; 7 and 8; 10 and 11; 13 and 14; 16 and 17; 19 and 20; 22 and 23; 25 and 26; 28 and 29; 31 and 32; 34 and 35; 37 and 38; 40 and 41; 43 and 44; 46 and 47; 49 and 50; 52 and 53; 55 and 56; 58 and 59; 61 and 62; 64 and 65; 67 and 68;
preferably, the kit further comprises one or more components selected from the following: an amplification primer set, a probe set, reagents for digital PCR;
preferably, the amplification primer set comprises at least one amplification primer (e.g., a pair of amplification primers or more amplification primers), which is capable of specifically amplifying a nucleic acid molecule containing the SNP site;
preferably, the probe set comprises a first probe and a second probe; wherein,
(i) the first probe and the second probe are each independently labeled with a reporter group and a quencher group, wherein the reporter group is capable of emitting a signal, and the quencher group is capable of absorbing or quenching the signal emitted by the reporter group; and, the first probe and the second probe are respectively labeled with different reporter groups (e.g., fluorophores); and
(ii) the first probe is capable of hybridizing or annealing (preferably completely complementary) to a nucleic acid molecule containing a first allele of the target SNP site, and the second probe is capable of hybridizing or annealing (preferably completely complementary) to a nucleic acid molecule containing a second allele of the target SNP site; and, the first probe and the second probe are specific for different alleles;
preferably, the probe set comprises probes having nucleotide sequences selected from the following or any combination thereof (e.g., any combination of 5, 10, 20, 40, 60): SEQ ID NOs: 73, 74, 78, 79, 82, 83, 86, 87, 90, 91, 94, 95, 98, 99, 102, 103, 106, 107, 110, 111, 114, 115, 118, 119, 122, 123, 126, 127, 130, 131, 134, 135, 138, 139, 142, 143, 146, 147, 150, 151, 154, 155, 158, 159, 162, 163;
preferably, the amplification primer set comprises a primer pair having nucleotide sequences selected from the following or any combination thereof (e.g., any combination of 5 pairs, 10 pairs, 15 pairs, 20 pairs, 23 pairs): SEQ ID NOs: 72 and 73; 77 and 76; 80 and 81; 84 and 85; 88 and 89; 92 and 93; 96 and 97; 100 and 101; 104 and 105; 108 and 109; 112 and 113; 116 and 117; 120 and 121; 124 and 125; 128 and 129; 132 and 133; 136 and 137; 140 and 141; 144 and 145; 148 and 149; 152 and 153; 156 and 157; 160 and 161;
preferably, the reagents for performing digital PCR are selected from one or more components selected from the following: a reagent for preparing droplet sample, a reagent for nucleic acid amplification, a nucleic acid polymerase, a reagent for detecting droplet sample, or any combination thereof,
preferably, the kit further comprises one or more components selected from the following: a nucleic acid polymerase, a reagent for nucleic acid amplification, a reagent for melting curve analysis, or any combination thereof;
preferably, the nucleic acid polymerase is a template-dependent nucleic acid polymerase, such as a DNA polymerase, especially a thermostable DNA polymerase; preferably, the nucleic acid polymerase is as defined in claim 24;
preferably, the reagent for nucleic acid amplification comprises a working buffer for enzyme (e.g., nucleic acid polymerase), dNTPs (labeled or unlabeled), water, solution containing ion (e.g., Mg²⁺), single-stranded DNA binding protein, or any combination thereof;
preferably, the kit is used to determine whether the recipient sample contains the donor, or to calculate the ratio of the donor in the recipient sample;
preferably, the digital PCR is selected from droplet digital PCR and chip digital PCR.

28. Use of the identification primer set as defined in claim 27 in the manufacture of a kit, wherein the kit is used for asymmetrically amplifying a target nucleic acid molecule, or for detecting a genotype of a candidate SNP site in a target nucleic acid molecule; or for identifying an SNP site with different genotypes between the donor and the recipient; or for identifying an SNP site with a homozygous allele in the recipient;
preferably, the kit further comprises the detection probe as defined in claim 27;
preferably, the kit is used for carrying out the method described in claim 1, 8 or 15.

29. Use of the amplification primer set and probe set as defined in claim 27 in the manufacture of a kit, wherein the kit is used for detecting the presence or proportion of a nucleic acid of the donor in a sample from the recipient who has undergone transplantation;
preferably, the kit further comprises a reagent for determining a genotype of one or more SNP sites in the genome of the recipient or the donor;
preferably, the kit further comprises the identification primer set and detection probe as defined in claim 27;
preferably, the kit is used for carrying out the method described in claim 2, 9 or 16.
